(19) United States
(12) Patent Application Publication
(10) Pub. No.: US 2023/0393151 A1
(43) Pub. Date: Dec. 7, 2023

(54) Precision Targeted Retromer Therapeutics for the Treatment of Neurodegenerative Diseases and Disorders

(71) Applicant: The Trustees of Columbia University in the City of New York, New York, NY (US)

(72) Inventors: Scott A. SMALL, New York, NY (US); Sabrina ALVES SIMOES SPASSOV, New York, NY (US); Yasir QURESHI, New York, NY (US)

(73) Assignee: The Trustees of Columbia University in the City of New York, New York, NY (US)

(21) Appl. No.: 18/006,212

(22) PCT Filed: Jul. 20, 2021
(86) PCT No.: PCT/US2021/042432

§ 371 Date: Aug. 11, 2023

Related U.S. Application Data
(60) Provisional application No. 63/053,805, filed on Jul. 20, 2020.
(60) Provisional application No. 63/090,477, filed on Oct. 12, 2020.

(51) Int. Cl. G01N 33/68; C12N 15/86; A61K 38/16; A61P 25/28; A61P 25/16

(52) U.S. Cl. CPC G01N 33/6896; C12N 15/86; A61K 38/16; A61P 25/28; A61P 25/16; G01N 2800/2821; G01N 2800/2835; C12N 2750/14143

(57) ABSTRACT

The present disclosure relates to methods and compositions for elevating and stabilizing retromer protein for treating and/or preventing neurodegenerative diseases and disorders including Alzheimer's disease and Parkinson's disease, where retromer dysfunction and/or endosomal trafficking dysfunction is involved and/or implicated in the disease or disorder. The methods include the use of agents to elevate and stabilize specific retromer depending upon the neurodegenerative disease or disorder being treated. The method can also include the use of one or more biomarkers to detect and/or identify whether retromer dysfunction and/or endosomal trafficking dysfunction is involved and/or implicated in the disease or disorder.

### CROSS-REFERENCE TO RELATED APPLICATIONS

The present application is a 371 of International Patent Application No. PCT/US2021/042432 filed Jul. 20, 2021, and claims priority to U.S. Provisional Patent Applications Nos. 63/053,805 filed Jul. 20, 2020, and 63/090,477 filed Oct. 12, 2020, each of which are incorporated herein by reference in their entirety.

### STATEMENT OF GOVERNMENTAL INTEREST

This invention was made with government support under U01AG024904, R01AG034618, R01AG035015 and P50AG008702 all awarded by the National Institutes of Health, and W81XWH-12-2-0012 awarded by the Department of Defense. The government 20 has certain rights in the invention.

### SEQUENCE LISTING

The instant application contains a Sequence Listing which has been submitted in text format via EFS-Web and is hereby incorporated by reference in its entirety. Said text copy, created on 25 Jan. 2023, is named 39700041US01SEQL and is 28,668 bytes in size.

### FIELD

The present disclosure relates to methods and compositions for elevating and stabilizing retromer protein for treating and/or preventing neurodegenerative diseases and disorders including Alzheimer's disease and Parkinson's disease, where retromer dysfunction and/or endosomal trafficking dysfunction is involved and/or implicated in the disease or disorder. The methods include the use of agents to elevate and stabilize specific retromer depending upon the neurodegenerative disease or disorder being treated. The method can also include the use of one or more biomarkers to detect and/or identify whether retromer dysfunction and/or endosomal trafficking dysfunction is involved and/or implicated in the disease or disorder.

### BACKGROUND

“Precision medicine”, the idea that a subset of patients with a particular cancer as well as a molecular signature will be most amenable to a particular therapy, has been used in the field of cancer therapeutics. The idea has not, to date, been applied in neurological diseases and disorders.

Genomic and cell biological studies have established that endosomal trafficking is a biological pathway linked to Alzheimer's disease (AD) pathogenesis (Small et al., 2017). Some of the best evidence in support of this conclusion is provided by molecules related to retromer (Small and Petsko, 2015). Retromer is a protein complex that has emerged as a master conductor of endosomal trafficking, by retrogradely transporting receptors from the endosomal compartment back to the trans-Golgi network, and by recycling receptors to the cell surface (Small and Petsko, 2015). The cargo recognition core is retromer's central module, so named because it is the core to which transported receptors bind, but also because it is with this core that other retromer modules interact (Seaman, 2012). This trimeric core includes the protein “Vacuolar Protein Sorting 35” (VPS35) to which the other core proteins VPS29 and VPS26 bind.

Neurodegeneration is the umbrella term for the progressive loss of structure or function of neurons, including death of neurons. There are a growing number of neurological/neurodegenerative diseases and disorders in which retromer is implicated. These disease and disorders include but are not limited to Alzheimer's disease (AD), Parkinson's disease (PD), Amyotrophic Lateral Sclerosis (ALS), Frontal-Temporal Degeneration, Downs Syndrome, and Prion Disease. For each, though, it is assumed that retromer is deficient in only a subset of patients. Thus, described herein is the use of precision medicine to identify and treat these patients by combining biomarkers of retromer dysfunction to first identify this subset, and then using the most appropriate retromer therapeutic that matches and targets the “retromer signature”.

### SUMMARY

Disclosed herein are methods for use in a precision medicine model for neurodegenerative disease or disorder including but not limited to Alzheimer's disease (AD), Parkinson's disease (PD), Amyotrophic Lateral Sclerosis (ALS), Frontal-Temporal Degeneration, Downs Syndrome, and Prion Disease.

Using the methods disclosed herein, a practitioner can detect and/or identify whether retromer dysfunction and/or endosomal trafficking dysfunction is involved and/or implicated in the neurodegenerative disease or disorder in a subject who is suffering from the neurodegenerative disease or disorder, or is suspected of having the neurodegenerative disease or disorder, or is at risk for the neurodegenerative disease or disorder, and then treating the subject accordingly with the appropriate retromer therapeutic.

In some embodiments, the method disclosed herein for treating a subject for a neurodegenerative disease or disorder comprises:
a. detecting or measuring the amount or level of one or more biomarkers in a sample from the subject, wherein the one or more biomarkers identify retromer dysfunction and/or endosomal trafficking dysfunction;
b. comparing the amount or level of the one or more biomarkers in the sample to a reference amount or level of one or more of the biomarkers, wherein if the amount or level of the one or more biomarkers in the sample is different than the reference amount or level of one or more biomarkers, the subject is suffering from a neurodegenerative disease or disorder where retromer dysfunction and/or endosomal trafficking dysfunction is involved and/or implicated, and wherein the reference amount or level is from a healthy control; and
c. treating the subject with the appropriate retromer therapeutic.

In further embodiments, the method disclosed herein for treating a subject for a neurodegenerative disease or disorder comprises:
a. detecting or measuring the amount or level of one or more biomarkers in a sample from the subject, wherein the one or more biomarkers identify retromer dysfunction and/or endosomal trafficking dysfunction;
b. comparing the amount or level of the one or more biomarkers in the sample to a reference amount or level of one or more of the biomarkers, wherein if the amount or level of the one or more biomarkers in the sample is the same as the reference amount or level of one or more biomarkers, the subject is suffering from a neurodegenerative disease or disorder where retromer dysfunction and/or endosomal trafficking dysfunction is involved and/or implicated, and wherein the reference amount or level is from a subject who is suffering from a neurodegenerative disease or disorder where retromer dysfunction and/or endosomal trafficking dysfunction is involved and/or implicated; and
c. treating the subject with the appropriate retromer therapeutic.

In some embodiments, the subject has already been diagnosed with the neurodegenerative disease or disorder. In some embodiments, the subject is suspected of having the neurodegenerative disease or disorder. In some embodiments, the subject has symptoms of a neurodegenerative disease or disorder. In some embodiments, the subject is at risk for the neurodegenerative disease or disorder.

In some embodiments, the biomarker is a nucleic acid. In some embodiments, the biomarker is a protein. In some embodiments, the method uses both nucleic acid biomarkers and protein biomarkers. In some embodiments, the biomarker is detected with an imaging method including but not limited to SPECT, PET and MRI imaging.

In some embodiments, the retromer therapeutic is a composition containing a nucleic acid or a transgene encoding one or more of the retromer proteins described herein. The composition may be a vector, for example, a viral vector, such as an adeno-associated virus (AAV) vector.

In some embodiments, the retromer therapeutic is a composition or compositions containing one or more pharmacological retromer chaperones.

In some embodiments, the retromer therapeutic is both a vector, for example, a viral vector, such as an adeno-associated virus (AAV) vector and one or more pharmacological retromer chaperones.

In some embodiments, the neurodegenerative disease or disorder is Alzheimer's disease (AD). As described herein, subjects with Alzheimer's disease would benefit most from the administration of a retromer therapeutic which is a composition containing a nucleic acid or a transgene encoding VPS35 alone or VPS26b alone, a composition containing a viral vector comprising a nucleic acid or a transgene encoding VPS35 alone or VPS26b alone, and/or a pharmacological chaperone that increases or stabilizes VPS35 and/or VPS26b.

Thus, a further embodiment of the present disclosure is a method disclosed herein for treating a subject for Alzheimer's disease comprising:
a. detecting or measuring the amount or level of one or more biomarkers in a sample from the subject, wherein the one or more biomarkers identify retromer dysfunction and/or endosomal trafficking dysfunction involvement and/or implication in Alzheimer's disease;
b. comparing the amount or level of the one or more biomarkers in the sample to a reference amount or level of one or more of the biomarkers, wherein if the amount or level of the one or more biomarkers in the sample is different than the reference amount or level of one or more biomarkers, the subject is suffering from Alzheimer's disease where retromer dysfunction is involved and/or implicated, and wherein the reference amount or level is from a healthy control; and
c. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, pharmacological chaperone which increases VPS35 and/or VPS26b, and combinations thereof.

Yet a further embodiment of the present disclosure is a method disclosed herein for treating a subject for Alzheimer's disease comprising:
a. detecting or measuring the amount or level of one or more biomarkers in a sample from the subject, wherein the one or more biomarkers identify retromer dysfunction and/or endosomal trafficking dysfunction involvement and/or implication in Alzheimer's disease;
b. comparing the amount or level of the one or more biomarkers in the sample to a reference amount or level of one or more of the biomarkers, wherein if the amount or level of the one or more biomarkers in the sample is the same as the reference amount or level of one or more biomarkers, the subject is suffering from Alzheimer's disease where retromer dysfunction and/or endosomal trafficking dysfunction is involved and/or implicated, and wherein the reference amount or level is from a subject suffering from Alzheimer's disease where retromer dysfunction and/or endosomal trafficking dysfunction is involved and/or implicated; and
c. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

In some embodiments, the biomarker is a nucleic acid. In some embodiments, the biomarker is a protein. In some embodiments, the method uses both nucleic acid biomarkers and protein biomarkers.

In some embodiments, the one or more biomarkers are selected from the group consisting of the N-terminal fragment of APLP1 (n-APLP1), the N-terminal fragment of CHL1 (n-CHL1), tau, and combinations thereof.

Thus, a further embodiment of the present disclosure is a method for treating a subject for Alzheimer's disease comprising:
a. purifying and/or isolating protein from a sample from the subject;
b. detecting or measuring the amount or level of the N-terminal fragment of CHL1;
c. comparing the amount or level of the N-terminal fragment of CHL1 from the sample to a reference amount or level of the N-terminal fragment of CHL1;
d. detecting or measuring the amount or level of the N-terminal fragment of APLP1;
e. comparing the amount or level of the N-terminal fragment of APLP1 from the sample to a reference amount or level of the N-terminal fragment of APLP1; and
f. detecting that the subject has retromer dysfunction and/or endosomal trafficking dysfunction when the amount or level of the N-terminal fragment of CHL1 and the amount or level of the N-terminal fragment of APLP1 are increased compared to the reference amount or level; and
g. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

A further embodiment of the present disclosure is a method for treating a subject for Alzheimer's disease comprising:
a. purifying and/or isolating protein from a sample from the subject;
b. detecting or measuring the amount or level of the N-terminal fragment of CHL1;
c. comparing the amount or level of the N-terminal fragment of CHL1 from the sample to a reference amount or level of the N-terminal fragment of CHL1;
d. detecting or measuring the amount or level of the N-terminal fragment of APLP1;
e. comparing the amount or level of the N-terminal fragment of APLP1 from the sample to a reference amount or level of the N-terminal fragment of APLP1;
f. detecting or measuring the amount or level of tau;
h. comparing the amount or level of tau from the sample to a reference amount or level of tau;
i. detecting that the subject has Alzheimer's disease where retromer dysfunction and/or endosomal trafficking dysfunction is involved and/or implicated when the amount or level of the N-terminal fragment of CHL1 and the amount or level of the N-terminal fragment of APLP and the amount or level of tau are increased compared to the reference amount or level; and
j. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

Yet a further embodiment of the present disclosure is a method for treating a subject for Alzheimer's disease comprising:
a. purifying and/or isolating protein from a sample from the subject;
b. detecting or measuring the amount or level of the N-terminal fragment of CHL1;
c. comparing the amount or level of the N-terminal fragment of CHL1 from the sample to a reference amount or level of the N-terminal fragment of CHL1;
d. detecting or measuring the amount or level of the N-terminal fragment of APLP1;
e. comparing the amount or level of the N-terminal fragment of APLP1 from the sample to a reference amount or level of the N-terminal fragment of APLP1; and
f. detecting that the subject has retromer dysfunction and/or endosomal trafficking dysfunction when the amount or level of the N-terminal fragment of CHL1 and the amount or level of the N-terminal fragment of APLP1 are the same as compared to the reference amount or level; and
g. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

A further embodiment of the present disclosure is a method for treating a subject for Alzheimer's disease comprising:
a. purifying and/or isolating protein from a sample from the subject;
b. detecting or measuring the amount or level of the N-terminal fragment of CHL1;
c. comparing the amount or level of the N-terminal fragment of CHL1 from the sample to a reference amount or level of the N-terminal fragment of CHL1;
d. detecting or measuring the amount or level of the N-terminal fragment of APLP1;
e. comparing the amount or level of the N-terminal fragment of APLP1 from the sample to a reference amount or level of the N-terminal fragment of APLP1;
f. detecting or measuring the amount or level of tau;
h. comparing the amount or level of tau from the sample to a reference amount or level of tau;
i. detecting that the subject has Alzheimer's disease where retromer dysfunction and/or endosomal trafficking dysfunction is involved and/or implicated when the amount or level of the N-terminal fragment of CHL1 and the amount or level of the N-terminal fragment of APLP and the amount or level of tau are the same compared to the reference amount or level; and
j. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

In some embodiments, the proteins are measured or detected using a Simoa™ assay.

In some embodiments, tau is phosphorylated. In some embodiments, tau is the mid-domain of tau (m-tau).

In some embodiments, the reference amount or level is from a healthy control.

In some embodiments, the reference amount or level is from a subject suffering from Alzheimer's disease where retromer dysfunction and/or endosomal trafficking dysfunction is involved and/or implicated.

In some embodiments, the subject has already been diagnosed with Alzheimer's disease. In some embodiments, the subject is suspected of having Alzheimer's disease. In some embodiments, the subject has one or more symptoms of Alzheimer's disease including but not limited to mild cognitive impairment (MCI). In some embodiments, the subject is at risk for Alzheimer's disease.

In some embodiments, the neurodegenerative disease or disorder is Parkinson's disease (PD). As described herein, subjects with Parkinson's disease would benefit most from the administration of a retromer therapeutic which is a composition containing a nucleic acid or a transgene encoding VPS35 alone or VPS26a alone, and/or a pharmacological chaperone that increases or stabilizes VPS35 and/or VPS26a.

Thus, a further embodiment of the present disclosure is a method for treating a subject for Parkinson's disease comprising:
a. detecting or measuring the amount or level of one or more biomarkers in a sample from the subject, wherein the one or more biomarkers identify retromer dysfunction and/or endosomal trafficking dysfunction involvement and/or implication in Parkinson's disease;
b. comparing the amount or level of the one or more biomarkers in the sample to a reference amount or level of one or more of the biomarkers, wherein if the amount or level of the one or more biomarkers in the sample is different than the reference amount or level of one or more biomarkers, the subject is suffering from Parkinson's disease where retromer dysfunction and/or endosomal trafficking dysfunction is involved and/or implicated, and wherein the reference amount or level is from a healthy control; and
c. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

Yet a further embodiment of the present disclosure is a method for treating a subject for Parkinson's disease comprising:
a. detecting or measuring the amount or level of one or more biomarkers in a sample from the subject, wherein the one or more biomarkers identify retromer dysfunction and/or endosomal trafficking dysfunction involvement and/or implication in Parkinson's disease;
b. comparing the amount or level of the one or more biomarkers in the sample to a reference amount or level of one or more of the biomarkers, wherein if the amount or level of the one or more biomarkers in the sample is the same as the reference amount or level of one or more biomarkers, the subject is suffering from Parkinson's disease where retromer dysfunction and/or endosomal trafficking dysfunction is involved and/or implicated, and wherein the reference amount or level is from a subject suffering from Parkinson's disease where retromer dysfunction and/or endosomal trafficking dysfunction is involved and/or implicated; and
c. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

In some embodiments, the biomarker is a nucleic acid. In some embodiments, the biomarker is a protein. In some embodiments, the method uses both nucleic acid biomarkers and protein biomarkers.

In some embodiments, the one or more biomarkers is the N-terminal fragment of APLP1 (n-APLP1).

Thus, a further embodiment of the present disclosure is a method for treating a subject for Parkinson's disease comprising:
a. purifying and/or isolating protein from a sample from the subject;
b. detecting or measuring the amount or level of the N-terminal fragment of APLP1;
c. comparing the amount or level of the N-terminal fragment of APLP1 from the sample to a reference amount or level of the N-terminal fragment of APLP1;
d. detecting that the subject has Parkinson's disease where retromer dysfunction and/or endosomal trafficking dysfunction is involved and/or implicated when the amount or level of the N-terminal fragment of APLP1 is increased compared to the reference amount or level; and
e. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

A further embodiment of the present disclosure is a method for treating a subject for Parkinson's disease comprising:
a. purifying and/or isolating protein from a sample from the subject;
b. detecting or measuring the amount or level of the N-terminal fragment of APLP1;
c. comparing the amount or level of the N-terminal fragment of APLP1 from the sample to a reference amount or level of the N-terminal fragment of APLP1;
d. detecting that the subject has Parkinson's disease where retromer dysfunction and/or endosomal trafficking dysfunction is involved and/or implicated when the amount or level of the N-terminal fragment of APLP1 is the same compared to the reference amount or level; and
e. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

In some embodiments, the reference amount or level is from a healthy control.

In some embodiments, the reference amount or level is from a subject suffering from Parkinson's disease where retromer dysfunction and/or endosomal trafficking dysfunction is involved and/or implicated.

In some embodiments, the subject has already been diagnosed with Parkinson's disease. In some embodiments, the subject is suspected of having Parkinson's disease. In some embodiments, the subject has one or more symptoms of Parkinson's disease including but not limited to: tremors, trembling of hands, arms, legs, jaw and face; stiffness of the arms, legs and trunk; slowness of movement; poor balance and coordination; and speech difficulty. In some embodiments, the subject is at risk for Parkinson's disease.

In the further embodiments, the disclosure provides for the use of a composition or compositions in the methods as described herein. As part of the foregoing aspects, the disclosure therefore provides the use of a composition containing a nucleic acid or transgene encoding retromer core protein VPS35 or VPS26a or VPS26b in treating, preventing, and/or curing a neurodegenerative disease or disorder and/or alleviating one or more symptoms associated with a neurodegenerative disease or disorder, wherein retromer dysfunction and/or endosomal trafficking dysfunction is involved and/or implicated in the neurodegenerative disease or disorder. In some embodiments, the nucleic acid or transgene encodes VPS35. In some embodiments, the nucleic acid or transgene encodes VPS26a. In some embodiments, the nucleic acid or transgene encodes VPS26b.

In some embodiments, the composition comprises a vector, such as a viral vector. The viral vector may be, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

In some embodiments, the viral vector is an AAV vector, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference). In certain embodiments, the viral vector is AAV10. For example, the composition may comprise AAV10 comprising a nucleic acid sequence or a transgene encoding retromer core protein VPS35 or retromer core protein VPS26a or a retromer core protein VPS26b.

In certain embodiments, the viral vector is AAV9. For example, the composition may comprise AAV9 comprising a nucleic acid sequence or a transgene encoding retromer core protein VPS35 or retromer core protein VPS26a or a retromer core protein VPS26b.

In certain embodiments, the viral vector is AAV2/10. For example, the composition may comprise AAV2/10 comprising a nucleic acid sequence or a transgene encoding retromer core protein VPS35 or retromer core protein VPS26a or a retromer core protein VPS6b.

In certain embodiments, the viral vector is AAV2/9. For example, the composition may comprise AAV2/9 comprising a nucleic acid sequence or a transgene encoding retromer core protein VPS35 or retromer core protein VPS26a or a retromer core protein VPS26b.

In certain embodiments, the viral vector is an AAV vector and the transgene is VPS35 retromer core protein. For example, the composition may comprise a recombinant AAV (rAAV), such as AAV10, comprising a nucleic acid sequence comprising a transgene encoding a functional VPS35 retromer core protein. For example, the composition may comprise a recombinant AAV (rAAV), such as AAV9, comprising a nucleic acid sequence comprising a transgene encoding a functional VPS35 retromer core protein. For example, the composition may comprise a recombinant AAV (rAAV), such as AAV2/9 or AAV2/10, comprising a nucleic acid sequence comprising a transgene encoding a functional VPS35 retromer core protein.

In certain embodiments, the viral vector is an AAV vector and the transgene is retromer core protein VPS26a. For example, the composition may comprise a recombinant AAV (rAAV), such as AAV10, comprising a nucleic acid sequence comprising a transgene encoding a functional VPS26a retromer core protein. For example, the composition may comprise a recombinant AAV (rAAV), such as AAV9, comprising a nucleic acid sequence comprising a transgene encoding a functional retromer core protein VPS26a. For example, the composition may comprise a recombinant AAV (rAAV), such as AAV2/9 or AAV2/10, comprising a nucleic acid sequence comprising a transgene encoding a functional retromer core protein VPS26a.

In certain embodiments, the viral vector is an AAV vector and the transgene is retromer core protein VPS26b. For example, the composition may comprise a recombinant AAV (rAAV), such as AAV10, comprising a nucleic acid sequence comprising a transgene encoding a functional VPS26b retromer core protein. For example, the composition may comprise a recombinant AAV (rAAV), such as AAV9, comprising a nucleic acid sequence comprising a transgene encoding a functional retromer core protein VPS26b. For example, the composition may comprise a recombinant AAV (rAAV), such as AAV2/9 or AAV2/10, comprising a nucleic acid sequence comprising a transgene encoding a functional retromer core protein VPS26b.

In some embodiments, the composition is a liposome, vesicle, synthetic vesicle, exosome, synthetic exosome, dendrimer, or nanoparticle.

In some embodiments, the transgene is operably linked to a promoter that induces expression of the transgene in a neuron. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the transgene is operably linked to an enhancer that induces expression of the transgene in a neuron. Exemplary enhancers that may be used in conjunction with the compositions and methods of the disclosure are a CMV enhancer, a myocyte enhancer factor 2 (MEF2) enhancer, and a MyoD enhancer.

As part of the foregoing aspects, the disclosure further provides the use of a composition containing a pharmacological retromer chaperone in the methods disclosed herein. Pharmacological retromer chaperones that can be used in the disclosed methods include but are not limited to small molecules and other agents including but not limited to chemicals, pharmaceuticals, biologics, antibodies, nucleic acids, peptides, and proteins. In some embodiments, the pharmacological chaperone binds at the interface between VPS35 and VPS29. Pharmacological chaperones that can used in the disclosed methods and compositions include but are not limited to R55 and R33.

The disclosure further provides for kits containing the compositions for use in the disclosed methods. The kit may further contain a package insert, such as a package insert instructing a user of the kit to administer the composition to a subject in accordance with the method of any of the above aspects or embodiments of the disclosure.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1. Proteomic CSF screen in Vps35 depleted mice. FIG. 1A is a schematic of the generation of neuronal-selective Vps35 knockout mice were generated by crossing mice expressing 1oxP-flanked Vps35 (Vps35^{fl/fl}) with mice expressing Cre recombinase under the Camk2a promoter. FIG. 1B are representative confocal images of 6 month-old mice brain sections stained for VPS35 Immunofluorescent images of the hippocampus show depletion of Vps35 in the neuron-selective knockout mice (right panel). Scale bar, 200 μm. FIG. 1C are graphs quantifying Western blot analysis of hippocampal tissue from Vps35 cKO and control littermates (Control) revealed for the retromer core proteins, VPS35, VPS26a and VPS29. n=4 animals/genotype. Data are shown as means±SEM. FIG. 1D is a schematic of the CSF proteomic workflow. Individual CSF samples (free of blood contamination) were pooled according to genotype, and the pooled samples were then analyzed by LC-MS/MS in technical duplicates. Significance is indicated as *P<0.05, **P<0.01, and ***P<0.005.

FIG. 2. Identifying alterations in CSF proteins in neuronal-selective Vps35 KO mice. FIG. 2A are tables of listed proteins that are significantly reduced or elevated in the CSF of Vps35 cKO mice compared with controls. The four BACE1 substrates, APLP2, CHL1, APLP1, and APP, are listed in the elevated proteins table. FIG. 2B are scatterplots showing the distribution of the four identified sBACE1 substrates: APLP2, CHL1, APLP1, and APP. Values are reported as label-free quantification (LFQ) intensity and represent the mean of technical duplicates. Bars indicate the means±SD; Limma-calculated q values are reported (n=3 to 4 per genotype; ***q<0.001 and **q<0.05). FIG. 2C is an illustration of how these substrates are cleaved at the endosomal membrane by BACE1, liberating N-terminal fragments into the endosomal lumen.

FIG. 3. Nonparametric and pathway analysis of CSF proteomic abnormalities caused by Vps35 depletion. FIG. 3A is a table of listed proteins detected only in the controls or in the Vps35 cKO mice, with microtubule-associated protein tau (MAPT) indicated in capital text. FIG. 3B shows Tau protein structural features in the linear diagram. Peptide region identified by MS/MS is indicated with an arrow. FIG. 3C is a scatterplot showing the P value distribution of over 800 proteins identified by pathway analysis as “regulator” proteins. FIG. 3D is a table of listed proteins that are the top six regulators, with lowest P values.

FIG. 4. Validating BACE1 substrates and Tau accumulation in mouse CSF. FIG. 4A is a graph quantifying immunoblotting with antibodies directed against the N-terminal fragments of APLP1 (n-APLP1) and CHL1 (n-CHL1) in the CSF of a mixed sex cohort of Vps35 cKO mice (light circles, right side) versus controls (dark circles, left side) (n=6 to 8 per genotype; *P<0.05). Data are shown as means±SD. FIG. 4B is a scatterplot showing the relationship between n-APLP1 and n-CHL1 in the CSF of Vps35 cKO mice (light circles) and controls (dark circles) (n=14, β=0.89, P<0.0001; right). FIG. 4C is a scatter plot showing relationship between n-APLP1 and n-CHL1 in the CSF of Vps35 cKO mice (light circles) and controls (dark circles) (n=14, r=0.89, P<0.0001, with a R²=0.787). FIG. 4D are scatter plots showing relationship between CADM4 and n-APLP1 (n=14, r=−0.377, P=0.184, with a R²=0.142), or between CADM4 and n-CHL1 (n=14, r=−0.111, P=0.706, with a R²=0.012) in the CSF of Vps35 cKO mice (light circles) and controls (dark circles). FIG. 4E are scatter plots showing relationship between TUBB3 and n-APLP1 (n=14, r=0.013, P=0.967 with a R²=0.00016), or between TUBB3 and n-CHL1 (n=14, r=−0.108, P=0.7262, with a R²=0.012) in the CSF of Vps35 cKO mice (light circles) and controls (dark circles). FIG. 4F are scatter plots showing relationship between TUBB2a and n-APLP1 (n=14, r=0.094, P=0.750 with a R2=0.008), or between TUBB2a and n-CHL1 (n=14, r=−0.117, P=0.689, with a R²=0.014) in the CSF of Vps35 cKO mice (light circles) and controls (dark circles). Of note, all technical replicates were used for the analysis. FIG. 4G shows the results of using a Simoa™ assay to measure md-Tau in postmortem CSF in a mixed sex cohort of Vps35 cKO mice (light circles, right side) versus controls (dark circles, left side) (n=5 to 9 per genotype; P<0.00001). Two-tailed unpaired Student's t test was used for the statistical analysis FIG. 4H shows the results of an analysis of antemortem CSF collected from females and males at 3 months (n=11 to 13 per genotype, P<0.0001) and 6 months (n=8 to 10 per genotype, P<0.0001) of age using an md-Tau Simoa™ assay. A two-way ANOVA Sidak's multiple comparisons test was used for the statistical analysis. Data are shown as means±SD. FIG. 4I is a graph quantifying Nissl staining analysis in the hippocampus of 3-month-old Vps35 cKO mice compared with their control littermates. n=6 animals per genotype (P=0.8182, in a nonparametric Mann-Whitney test). FIG. 4J is a graph quantifying NeuN staining analysis in the hippocampus of 3-month-old Vps35 cKO mice compared to their control littermates (P=0.6991, in a non-parametric Mann Whitney test. n=6 animals/genotype). FIG. 4K are graphs quantifying Western blot analysis of hippocampal tissue from 3-month-old Vps35 cKO and control littermates (control) showing expression of VPS35, VPS26a and VPS29. FIG. 4L are graphs quantifying Western blot analysis of hippocampal tissue from 3-month-old Vps35 cKO and control littermates (control) showing expression of neurotubulin (P=0.0001), presynaptic (PSD95, P=0.8319), or postsynaptic (synaptophysin, P=0.3558) markers. Data are shown as means±SEM. n=6 animals per genotype. Two-tailed unpaired Student's t test was used for the statistical analysis. Data are shown as means±SEM. Significance is indicated as *P<0.05, **P<0.01, and ***P<0.005.

FIG. 5. Development and qualification of Simoa™ APLP1 and CHL1 assays. A panel of 8 commercial antibodies raised against human APLP1 were screened in all configurations against recombinant APLP1 protein on Simoa™ HD-1 platform (FIGS. 5A and 5B). The most sensitive assay (2×10 aka n-APLP1) was optimized; a standard curve range is shown in FIG. 5A and dilution linearity with healthy control CSF is shown in FIG. 5B. A panel of 6 commercial antibodies raised against human CHL1 were screened in all configurations against recombinant CHL1 protein on Simoa™ HD-1 platform (FIGS. 5C and 5D). The most sensitive assay (12×9 aka n-CHL1) was optimized; standard curve range is shown in FIG. 5C and dilution linearity with healthy control CSF are shown in FIG. 5D.

FIG. 6. The relationship of n-APLP1, n-CHL1, and md-Tau is selectively correlated in the CSF of patients with Alzheimer's patients with Alzheimer's dementia. FIG. 6A is a scatterplot showing the correlations between n-APLP1 and n-CHL1 (n=316; (3=0.72, P=9.1×10⁻⁴⁸) in the CSF of patients with mild to moderate AD. FIG. 6B is a scatterplot showing the correlation between n-APLP1 and md-Tau (n=316; β=0.6, P=1.3×10⁻³⁰) in the CSF of patients with mild to moderate AD. FIG. 6C is a scatterplot showing the correlation between n-CHL1 and md-Tau (n=316; β=0.53, P=1.7×10⁻³³) in the CSF of patients with mild to moderate AD. FIG. 6D is a scatterplot showing the correlations between Aβ42 and mid-Tau-APLP1 in amyloid-negative (blue circles) and amyloid-positive (red circles) individuals. FIG. 6E is a scatterplot showing the correlation between n-APLP1 and md-Tau in amyloid-negative (blue circles) and amyloid-positive (red circles) individuals. FIG. 6F is a scatterplot showing the correlation between n-CHL1 and md-Tau in amyloid-negative (blue circles) and amyloid-positive (red circles) individuals.

FIG. 7. The relationship of n-APLP1, n-CHL1, and md-Tau in the CSF of healthy controls and patients with MCI. FIG. 7A is a scatterplot showing the relationship between n-APLP1 and n-CHL1 (n=40; β=0.97, P=2.4×10⁻²⁴) in the CSF of healthy controls. FIG. 7B is a scatterplot showing the relationship between n-APLP1 and md-Tau (n=40; β=0.86, P=1.7×10⁻¹²) in the CSF of healthy controls. FIG. 7C is a scatterplot showing the relationship between n-CHL1 and md-Tau (n=40; β=0.87, P=2.1×10⁻¹⁷) in the CSF of healthy controls. FIG. 7D is a scatterplot showing the relationship between n-APLP1 and n-CHL1 (n=21; β=P=6.5×10⁻¹³) in the CSF of patients with MCI. FIG. 7E is a scatterplot showing the relationship between n-APLP1 and md-Tau in the CSF of patients with MCI. FIG. 7F is a scatterplot showing the relationship between n-CHL1 and md-Tau in the CSF of patients with MCI. FIG. 7G is a scatterplot showing the relationship between n-APLP1 and n-CHL1 (healthy control, blue circles; MCIs, red circles) after adjustment for CSF Aβ42. FIG. 7H is a scatterplot showing the relationship between n-APLP1 and md-Tau (middle; n=21; β=0.67, P=0.001) after adjustment for CSF Aβ42. FIG. 7I is a scatterplot showing the relationship between n-CHL1 and md-Tau (bottom; n=21; β=0.79, P=0.00002) after adjustment for CSF Aβ42.

FIG. 8. n-APLP1, n-CHL1, and md-Tau in the CSF of healthy controls and patients with prodromal AD. A set of 39 healthy controls and 19 prodromal AD classified on the basis of Tau/Aβ42 cutoff for AD (≥0.15) were analyzed using the n-APLP1 (FIG. 8A), n-CHL1 (FIG. 8B), and md-Tau (FIG. 8C) assays. Shown is an in-between group comparison analysis in the corrected concentrations of n-APLP1 (F=84.2, P=9.4×10⁻¹³) (FIG. 8A) and n-CHL1 (F=78.2, P=3.2×10⁻¹²) (FIG. 8B) in patients with prodromal AD versus healthy controls.

FIG. 9. n-APLP1 and n-CHL1 are correlated with phosphorylated Tau in the CSF of healthy controls and patients with AD. FIG. 9A is a scatterplot showing the relationship between n-APLP1 and p-tau217 in the CSF of patients with mild to moderate AD (n=316; β=0.36, P=6.5×10⁻¹¹). FIG. 9B is a scatterplot showing the relationship between n-APLP1 and p-tau217 in the CSF of healthy controls (n=37; β=0.72, P=5.1×10⁻⁷). FIG. 9C is a scatterplot showing the relationship between n-APLP1 and p-tau217 in the CSF of MCIs adjusted for CSF Aβ42 (n=21; β=0.63, P=0.002). FIG. 9D is a scatterplot showing the relationship between n-CHL1 and p-tau217 in the CSF of patients with mild to moderate AD (n=316; β=0.37, P=6.4×10⁻¹¹). FIG. 9E is a scatterplot showing the relationship between n-CHL1 and p-tau217 in the CSF of healthy controls (n=37; β=0.62, P=5.0×10⁻⁶). FIG. 9F is a scatterplot showing the relationship between n-CHL1 and p-tau217 in the CSF of MCIs adjusted for CSF Aβ42 (n=21; β=0.71, P=0.0003).

FIG. 10. VPS26b is highly expressed in brain tissue and primary neurons. FIG. 10A shows the expression levels of retromer protein VPS26b in in a range of mouse tissue samples (n=7), using β-actin as a loading control. FIG. 10B shows the expression levels of retromer protein VPS26a in in a range of mouse tissue samples (n=7), using β-actin as a loading control. FIG. 10C shows Western blot analysis of retromer proteins in different primary cultures, mouse and human cell lines, showing enrichment of VPS26b in primary neuronal cultures. FIG. 10D is a bar diagram showing mRNA expression levels of VPS26b and VPS26a in astrocytes, neurons, microglia and endothelial cells. Data are from an RNA-sequencing transcriptome and splicing database reported by Zhang et al., 2014. RNA expression was measured in fragments per kilobase of transcript sequence per million mapped fragments (FPKM).

FIG. 11. VPS26b and VPS26a define distinct retromer cores. FIG. 11A are images of co-immunoprecipitation analysis of retromer proteins extracted from primary neuronal cultures using VPS35 (10 μg; left panel), VPS26a (10 μg; middle panel) and VPS26b (10 μg; right panel) as baits support the hypothesis that the VPS26 paralogs form two separate retromer cores. FIG. 11B shows a graph of the quantitative colocalization studies, based on Pearson's correlation, which reveal that VPS35 shows higher percent colocalization with each VPS26 paralog (n=28-29 cells, from three independent experiments). FIG. 11C shows the amount of VPS26b in primary cultures derived from VPS26b heterozygote (‘HET’, n=6) or VPS26b KO mice (n=6) compared to VPS26b WT mice (n=8). FIG. 11D shows the amount of VPS26a in primary cultures derived from VPS26b heterozygote (‘HET’, n=6) or VPS26b KO mice (n=6) compared to VPS26b WT mice (n=8). FIG. 11E shows the amount of VPS29 in primary cultures derived from VPS26b heterozygote (‘HET’, n=6) or VPS26b KO mice (n=6) compared to VPS26b WT mice (n=8). FIG. 11F shows the amount of VPS35 in primary cultures derived from VPS26b heterozygote (‘HET’, n=6) or VPS26b KO mice (n=6) compared to VPS26b WT mice (n=8). FIGS. 11C-11F are quantitated from immunoblots (in a one-way ANOVA with Tukey's post hoc tests two-sided analysis) and support for VPS26b forming a distinct retromer core as shown by how a primary depletion of VPS26b has no effects on VPS26a but causes a secondary reduction in VPS29 and VPS35. FIG. 11G shows the amount of VPS26a in infecting neurons from VPS26a^{flox/flox} mice with a lentivirus expressing Cre recombinase (Cre, n=11), compared to neurons infected with a lentivirus expressing a catalytically dead Cre recombinase (ΔCre, n=12). FIG. 11H shows the amount of VPS26b in infecting neurons from VPS26a^{flox/flox} mice with a lentivirus expressing Cre recombinase (Cre, n=11), compared to neurons infected with a lentivirus expressing a catalytically dead Cre recombinase (ΔCre, n=12). FIG. 11I shows the amount of VPS29 in infecting neurons from VPS26a^{flox/flox} mice with a lentivirus expressing Cre recombinase (Cre, n=11), compared to neurons infected with a lentivirus expressing a catalytically dead Cre recombinase (ΔCre, n=12). FIG. 11J shows the amount of VPS35 in infecting neurons from VPS26a^{flox/flox} mice with a lentivirus expressing Cre recombinase (Cre, n=11), compared to neurons infected with a lentivirus expressing a catalytically dead Cre recombinase (ΔCre, n=12). FIGS. 11G-J are quantitated from immunoblots. Statistical analyses were performed using either unpaired student's t-test, with Welch's correction when required, or non-parametric Mann-Whitney t-test. (P=0.1480, in an unpaired t-test with Welch's correction) and show support for VPS26a forming a distinct retromer core by how a primary depletion of VPS26a, induced by infecting neurons from VPS26a^{flox/flox} mice with a lentivirus expressing Cre recombinase, compared to neurons infected with a lentivirus expressing a catalytically dead Cre recombinase, has no effect on VPS26b, but causes a secondary reduction in VPS29 and VPS35. FIG. 11K is a graph quantifying Western blots of hippocampus homogenates from VPS26a heterozygous (HET) mice (VPS26a WT, n=4; VPS26a HET, n=11) probed for retromer proteins. FIG. 11L is a graph quantifying Western blots of hippocampus homogenates from VPS26b heterozygous (HET) mice (VPS26b WT, n=7; VPS26b HET, n=7) probed for retromer proteins. The quantitative analysis of the Western blots shown in FIGS. 11K and 11L shows that a primary deficiency in VPS26a (P=0.0015) results in a secondary reduction VPS35 (P=0.0037) and VPS29 (P=0.0061), but not VPS26b (P=0.8101); while a primary deficiency in VPS26b (P<0.0001) results in a secondary reduction in VPS35 (P=0.0004) and VPS29 (P=0.0009), but not VPS26a (P=0.6373), arguing in favor of separate retromer cores. All statistical analysis were performed using Student's t-test besides for VPS26a in the VPS26a WT vs. VPS26a HET analysis. Values denote mean±S.E.M., where *P<0.05, **P<0.01 and ***P<0.001.

FIG. 12. VPS26b redistributes to recycling endosomes during neuronal stimulation. FIG. 12A shows the subcellular distribution of both VPS26 paralogs performed using confocal microscopy quantifications based on Pearson's correlation coefficient obtained by analysis of cells per group/condition. Kruskal-Wallis test with a Dunn's post hoc test was used for the statistical analysis. Note that while VPS26a is broadly detected in all different compartments, VPS26b is highly enriched in early (EEA1) and recycling (Syntaxin13 and pulse-chase Transferrin) endosomes, with less presence in the Trans-Golgi network (Golgin97) and late endosomes (Rab7). Data expressed as mean±S.E.M. *P<0.05, **P<0.01 and ***P<0.001. FIG. 12B shows the subcellular distribution of both VPS26 paralogs performed using immunogold labeling quantifications (% of gold particles) for VPS26b and VPS26a on ultrathin cryosections. Localization of an equivalent number of gold particles in each sample was assessed relative to the indicated cell compartment. Note that whereas the bulk of VPS26b localizes to tubular-vesicular structures, found at the vicinity of endosomes, VPS26a is broadly distributed among the different compartments. FIG. 12C shows line graphs of colocalization studies of the VPS26 paralogs based on Pearson's correlation coefficient (n=31-41 cells per condition, from four independent experiments). Primary hippocampal neurons were stimulated with glycine for 5 minutes to induce cLTP and the subcellular distribution of VPS26b and VPS26a was assessed by confocal microscopy, using markers of early (EEA1) and recycling (Syntaxin13 and pulse-chase transferrin) endosomes. Compared to basal conditions, cLTP caused VPS26b to increase its colocalization with markers of the recycling endosomes (in an ANOVA analysis; Pulse-chase transferrin: F(1,33)=20.7, P=6.9E-5; Syntaxin13: F(1,36)=38.8, P=3.5E-7) and caused VPS26a to increase its colocalization with a marker of early endosomes (EEA1: F(1,33)=24.5, P=2.1E-5). FIG. 12D is an illustration of the changes in distribution observed post stimulation for both VPS26 paralogs.

FIG. 13. The trans-entorhinal cortex differentially depends on VPS26b. FIG. 13A shows a region of interest (ROI) analysis of the relative CBV (rCBV) at the trans-entorhinal cortex (TEC) between VPS26b WT and VPS26b KO revealed a significant age-dependent worsening of rCBV in the TEC region (in an ANOVA analysis of the genotype×age interaction: F(3,38)=16.08, P=0.0003; n=9-10 animals/genotype). FIG. 13B shows results of electrophysiology—the mean fEPSP slopes, expressed as percentage of baseline measured before and after high frequency stimulation in the TEC in VPS26b WT and VPS26b KO. FIG. 13C shows results of electrophysiology—the mean fEPSP slopes, expressed as percentage of baseline measured before and after high frequency stimulation in the MEC in VPS26b WT and VPS26b KO. FIG. 13D shows results of electrophysiology—the mean fEPSP slopes, expressed as percentage of baseline measured before and after high frequency stimulation in the TEC in VPS26b WT and VPS26b HET and VPS26a HET. FIGS. 13B-13D show that 12-14 months VPS26b KO mice, compared to WT littermates (n=6 per group,) have LTP defects in the TEC (F(1,14)=69.2; P<0.001), but normal LTP in the MEC (F(1,14)=0.93; P=0.365), in a repeated-measures ANOVA post hoc Tukey. Abnormal LTP was also found in the TEC of 18 months VPS26b HET mice, but not in 18 months VPS26a HET mice: VPS26b HET vs. WT: F(2,11)=3.27; P=0.005; VPS26a HET vs. WT: P=0.42 (post 434 hoc Tukey) FIG. 13E shows coregistered brain slices of GluA1 immunostainings, from 14 months VPS26b WT mice (n=5) (left panel) and VPS26b KO mice (n=5) (middle panel), a pixel-based analysis (‘VPS26b WT vs. KO’ (right panel), showed that VPS26b KO mice have focal TEC reductions in GluA1 immunostaining levels (indicated by the red arrow) (right panel), in two-sample t-test; pixel-wise P<0.001; color bar represents t-values. Scale bar, 500 μm.

FIG. 14. Cognitive profiling supports VPS26b's regional association. FIG. 14A shows the results of VPS26b mice tested in the novel-object recognition (NOR) tasks at 3 time points. Data is expressed as mean±S.E.M. A two-way ANOVA with Bonferroni's post hoc test was used for the analysis. In the NOR task targeting the perirhinal cortex, (n=10 animals for each of the 6 independent groups), a defect in memory performance was observed only in the older group (12-14 months). FIG. 14B shows the results of VPS26b mice tested in the object-context recognition (OCR) tasks at 3 time points. Data is expressed as mean±S.E.M. In the entorhinal-sensitive OCR task (n=10-14 for each of the 6 independent groups), a two-way ANOVA with Bonferroni's post hoc corrections revealed a significant genotype X age interaction that was driven by age-related worsening in the VPS26b KO mice: F(2,67)=13.92, P<0.001. FIG. 14C shows the discrimination index and shows that OCR defects were also found in 12-14 months VPS26b HET mice vs. their WT littermates (n=9-14 per independent group; P=0.0016, in unpaired non-parametric Mann-Whitney t-test), but not in 12-14 months VPS26a HET mice vs. their WT littermates (n=8 per group; P=0.5077, in unpaired two-sided student's t-test).

FIG. 15. VPS26b mediates glutamate-receptor trafficking from the recycling endosome and only VPS26b depletion, but not VPS26a deficiency, results in a reduction of cell surface GluA1 levels. FIG. 15A is a graph of the quantification of cell surface GluA1 in cells deficient for VPS26b compared to VPS26b WTs (P=0.001), in a non-parametric Mann Whitney test. (n=11 biological replicates, from 4 independent experiments). The biotinylation experiments revealed a significant reduction in cell surface GluA1. FIG. 15B is a graph of the quantification of cell surface GluA1 in cells deficient for VPS26a compared to VPS26a WTs. Note that VPS26a deficiency does not have any impact on GluA1 surface levels (P=0.6661), in unpaired two-sided student's t-test. (n=5 biological replicates, from 2 independent experiments). The ratio of surface to total GluA1 is shown. The WT VPS26 ratios were set to 1, and all other values were calculated relative to it. Data expressed as mean±S.E.M. *P<0.05, **P<0.01 and ***P<0.001. FIG. 15C shows the quantitative colocalization analysis of GluA1 with early and recycling markers, based on Pearson's correlation coefficients, which revealed that VPS26b depletion results in an accumulation of GluA1 in recycling endosomes (Transferrin: P<0.0001; and Syntaxin13: P<0.0001) and to a less extend in early endosomes (EEA1: P=0.032), in a non-parametric Kruskal-Wallis with Dunn's post hoc test (n=27-37 cells per group/condition, from three separate cultures). FIG. 15D shows the summary of results of cell surface GluA1 levels assessed by confocal microscopy in non-permeabilized cultured neurons incubated with an N-terminal GluA1 antibody in three conditions: VPS26b WT neurons infected with lentivirus expressing GFP alone (‘VPS26b WT+GFP vector’); VPS26b KO neurons infected with lentivirus expressing GFP alone (‘VPS26b KO+GFP vector’); and VPS26b KO neurons infected with lentivirus expressing VPS26b-GFP (‘VPS26b KO+VPS26b-GFP’). Mean fluorescence intensity values revealed that VPS26b repletion fully restored GluA1 surface localization in VPS26b KO neurons, as summarized in the bar graph (P<0.0001), in a non-parametric Kruskal-Wallis with Dunn's post hoc test. (n=25-26 neurons/condition, from four independent experiments). Data expressed as mean±S.E.M., *P<0.05, **P<0.01 and ***P<0.001.

FIG. 16. VPS26b is the AD-targeted trans-entorhinal cortex. FIG. 16A shows 3D cortical surface. Cortical map. A three-dimensional rendering of the cortical surface, parcellated into color-coded cortical regions, shown in a medial view (first panel). Cortical flat-map. The three-dimensional surface of a unilateral cortical surface is shown flattened, rendered as a ‘flat-map’, so that all cortical regions can be viewed in a single snapshot. Individual cortical regions are color-coded, as labeled (second panel). Raw t-value map comparing AD vs. controls. A color-coded map of t-values was generated, comparing the cortical thickness of AD vs. controls, covarying for sex and age (color bar represents t-values of the between-group comparisons; the dashed line indicates the threshold t=12 to generate the thresholded t-value map) (third panel). Thresholded t-value map comparing AD vs. controls. By thresholding the t-value map, the most reliable cortical thinning in AD vs. controls is localized to the vicinity of the TEC (forth panel). FIG. 16B shows the Alzheimer's-targeted trans-entorhinal cortex (TEC) was isolated by performing a cortical thickness analysis of MRI's generated from Alzheimer's disease (AD) patients and 169 healthy controls. Its longitudinal extent is indicated by the yellow arrows, where the entorhinal cortex abuts the amygdala (the highlighted blue region). Pixels with the most reliable volumetric loss compared to controls are indicated in yellow/red (the color bar represents t-values of the between-group comparisons), and the TEC defect is also shown on a coronal MRI slice (right panel). FIG. 16C shows a representative human postmortem brain slice, matching the precise anatomical coordinates of the neuroimaging finding (left panel), with the subregions of the entorhinal cortex harvested for protein measurements shown in higher magnification (right panel): The TEC, the lateral EC (LEC), the intermediate EC (IEC) and the medial EC (MEC). FIG. 16D is a bar graph showing that in healthy controls (n=16), among the four retromer core proteins, the TEC was found differentially enriched in VPS26b (P<0.0001), in a non-parametric Kruskal-Wallis with Dunn's post hoc test, showing mean levels normalized to α-tubulin. FIG. 16E is a bar graph showing an ANOVA analysis revealed that, compared to age-matched healthy controls (n=9), AD brains (n=8) showed the most reliable reduction in the TEC's VPS26b (F(1,16)=12.96, P=0.002), showing normalized means. Data expressed as mean±S.E.M. *P<0.05, **P<0.01 and ***P<0.001.

FIG. 17. VPS26b KO Mice are Rescued with AAV9-VPS26b. FIG. 17A is a schematic representation of the mouse brain showing the injection site in the lateral recess of the lateral ventricle. Image credit: Allen Institute. Allen Adult Mouse Brain Anatomic Reference Atlas. FIG. 17B is an image of a Western blot of the entorhinal cortex of WT (C57BL/6J) mice, injected with AAV9-VPS26b at different doses and volume at 3 months, and harvested at 4 months of age. FIG. 17C shows VPS26b rescue in EC of VPS26b KO mice. The figure is a Western blot of mice injected at −3.7 months of age and brains harvested at −7.7 months. FIG. 17D shows VPS26b mice tested in the OCR task. Data is expressed as mean±S.E.M. As expected, a defect in memory performance was observed in the VPS26b KO mice compared to VPS26b WT controls both injected with AAV9-GFP (n=4-6 per group, P=0.0028, in unpaired two-sided student's t-test). An OCR behavioral rescue was observed in VPS26b KO mice injected with AAV9-VPS26b compared to VPS26b KO mice injected with AAV9-GFP alone (n=4-6 animals/group, P=0.0044, in a one-way ANOVA with Tukey's post hoc test (two-sided). *P<0.05, **P<0.01 and ***P<0.001. FIG. 17E. Mean fEPSP slopes, expressed as a percentage of baseline measured before and after high-frequency stimulation in the TEC region of VPS26b WT—AAV9-GFP (n=5); VPS26b KO—AAV9-GFP (n=6), and VPS26b KO—AAV9-VPS26b (n=6) mice. In a two-way repeated measured ANOVA test with a Dunnett's post hoc, a rescue of LTP defects is observed in VPS26b KO mice injected with AAV9-VPS26b-GFP compared to VPS26b KO mice injected with AAV9-GFP (VPS26b KO—AAV9-VPS26b-GFP: 151.6%±0.40%, vs. VPS26b KO-AAV9-GFP: 103.6%±0.95%, vs. VPS26b WT-AAV9-GFP: 178.3%±1.08%, F(2,14)=6.427, P=0.0105).

FIG. 18. N-APLP1 is elevated in the CSF of a mouse model of Parkinson's disease. FIG. 18A shows a Western blot of CSF from VPS35 D620N and WT littermate mice probed with APLP1 and albumin antibodies. FIG. 18B shows a Ponceau-S stain to check for consistency of total protein loaded onto the gel. FIG. 18C is a graph showing the quantification of APLP1 signal normalized to albumin showing an increase of APLP1 in heterozygous mutant mice for VPS35 D620N Parkinson disease variant.

FIG. 19 shows a schematic of the retromer core protein.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “subject” as used in this application refers to animals in need of therapeutic or prophylactic treatment. Subjects include mammals, such as canines, felines, rodents, bovine, equines, porcines, ovines, and primates. Thus, the compositions and methods can be used in veterinary medicine, e.g., to treat companion animals, farm animals, laboratory animals in zoological parks, and animals in the wild. The compositions and methods disclosed herein are particularly desirable for human medical applications.

The term “patient” as used in this application means a human subject. In some embodiments, the “patient” is known or suspected of having a neurodegenerative disease or disorder including but not limited to Alzheimer's disease (AD), Parkinson's disease (PD), Amyotrophic Lateral Sclerosis (ALS), Frontal-Temporal Degeneration, Downs Syndrome, and

Prion Disease.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small.

The term “in need thereof” would be a subject known or suspected of having or being at risk of having a neurodegenerative disease or disorder including but not limited to Alzheimer's disease (AD), Parkinson's disease (PD), Amyotrophic Lateral Sclerosis (ALS), Frontal-Temporal Degeneration, Downs Syndrome, and Prion Disease. In some embodiments, the subject has a neurodegenerative disease or disorder where retromer dysfunction and/or endosomal trafficking dysfunction is involved and/or implicated.

The term “agent” as used herein means a substance that produces or is capable of producing an effect and would include, but is not limited to, vectors, chemicals, pharmaceuticals, biologics, small organic molecules, antibodies, nucleic acids, peptides, and proteins.

The term “retromer” as used herein primarily means the retromer core complex (VPS35, VPS29, VPS26a, and VPS26b), but, can also extend more broadly to the endosomal trafficking pathways that retromer serves.

A “retromer therapeutic” is an agent or a drug which enhances or increases retromer function by increasing retromer levels (for example, a pharmacological retromer chaperone or a viral vector comprising a transgene).

As used herein, the term “pharmacological retromer chaperone” means small molecules or other agents that bind to a protein or to a complex of more than one protein, and by virtue of stabilizing the protein's three-dimensional structure, protect it from degradation and increase its steady-state concentration in the cell. In the context of this disclosure, the protein is retromer or one or more of its component proteins. See Mecozzi et al. 2014.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

The terms “identification”, “identify”, “identifying” and the like as used herein means to recognize a disease state or a clinical manifestation or severity of a disease state in a subject or patient. The term also is used in relation to test agents and their ability to have a particular action or efficacy.

The term “reference amount” or “reference level” as used herein can mean an amount or a quantity of a particular protein or nucleic acid in a sample from a healthy control. A “reference amount” or “reference level” may also mean an amount or a quantity of a particular protein or nucleic acid in a sample from a patient at another time point in the disease and/or treatment. A “reference amount” or “reference level” may also mean an amount or a quantity of a particular protein or nucleic acid in a sample from a patient with a neurodegenerative disease or disorder where retromer dysfunction and/or endosomal trafficking dysfunction is involved and/or implicated.

The term “healthy control” is a human subject who is not suffering from a neurodegenerative disease or disorder where retromer dysfunction and/or endosomal trafficking dysfunction is involved and/or implicated.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

In some aspects, the disclosure provides isolated adeno-associated viral vectors (AAVs). As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities.

Methods for obtaining recombinant AAVs having a desired capsid protein have been described (see, for example, U.S. Pat. No. 7,906,111). A number of different AAV capsid proteins have been described, for example, those disclosed in Gao, et al., J. Virology 78(12):6381-6388 (June 2004); Gao, et al., Proc Natl Acad Sci USA 100(10):6081-6086 (May 13, 2003); and U.S. Pat. Nos. 7,906,111; 8,999,678. In embodiments for the desired packaging of the presently described constructs and methods, the recombinant AAV may be AAV9 or AAV10 vector and capsid. However, it is noted that other suitable AAVs such as rAAVrh.8 and rAAVrh.10, or other similar vectors may be adapted for use in the present methods and compositions. Typically the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., Fisher et al, J. Virology 70:520-532 (1993) and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

As used herein, the terms “AAV1,” “AAV2,” “AAV3,” “AAV4,” and the like refer to AAV vectors containing ITRs from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAV vectors. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

Standard methods in molecular biology are described Sambrook, Fritsch and Maniatis Molecular Cloning, A Laboratory Manual, Cold Spring Harbor Laboratory Press, Cold Spring Harbor, NY (1982 & 1989 2nd Edition, 2001 3rd Edition); Sambrook and Russell Molecular Cloning, 3rd ed., Cold Spring Harbor Laboratory Press, Cold Spring Harbor, N Y (2001); Wu Recombinant DNA, Vol. 217, Academic Press, San Diego, CA) (1993). Standard methods also appear in Ausbel, et al. Current Protocols in Molecular Biology, Vols. 1-4, John Wiley and Sons, Inc. New York, NY (2001).

### Abbreviations

AD—Alzheimer's disease
PD—Parkinson's disease
MCI—mild cognitive impairment
VPS—Vacuolar Protein Sorting
TEC—trans-entorhinal cortex
MEC—medial entorhinal cortex
LEC—lateral entorhinal cortex
IEC—intermediate entorhinal cortex
HCs— healthy controls

### Precision Medicine for Neurodegenerative Diseases and Disorders

Disclosed herein are methods using “precision medicine” to identify and accordingly treat patients with neurodegenerative diseases and disorders.

“Precision medicine” refers to the logic used in cancer therapeutics: that only a subset of patients with a particular cancer has a particular molecular signature, thus suggesting that subset will be most amenable to a particular therapy.

There are a growing number of neurologic diseases in which retromer is implicated. For each, it is assumed that retromer is deficient in only a subset of patients—including a subset of patients with Alzheimer's disease, Parkinson's disease, Amyotrophic Lateral Sclerosis, Frontal-Temporal Degeneration, Down's Syndrome, and Prion Disease. The essence of precision medicine is to combine biomarkers of retromer dysfunction and/or endosomal trafficking dysfunction to first identify this subset, and then to use the most appropriate retromer therapeutic that matches the “retromer signature’.

Shown herein is this concept as applied to Alzheimer's disease and Parkinson's disease, although the concept can be broadened to all neurodegenerative diseases and disorders where retromer dysfunction and/or endosomal trafficking dysfunction is involved and/or implicated.

As shown herein, is a biofluidic biomarker for AD-associated retromer dysfunction. Also shown herein is evidence that drugs which increase the levels of VPS35 alone, or VPS26b alone, will be most effective.

Also as shown herein is a biofluidic biomarker for PD-associated retromer dysfunction. Also shown herein is evidence that drugs which increase the levels of VPS35 alone, or VPS26a alone, will be most effective.

### Retromer Protein

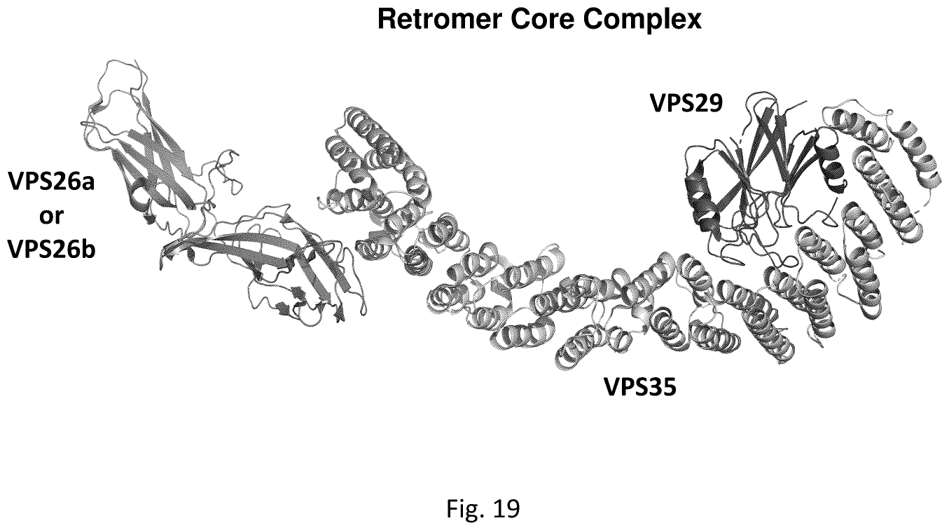

Retromer is a multiprotein complex that is a ‘master conductor’ of endosomal trafficking. Retromer's core is a trimer of three different proteins, making it technically a heterotrimer. The proteins are all members of the “Vacuolar Protein Sorting” (VPS) family of proteins. VPS35 is the trimer core's central protein, to which VPS29 and VPS26 bind. VPS26 is the only core protein that has two paralogs, called VPS26a and VPS26b. Thus, neurons have two distinct retromer cores-VPS29-VPS35-VPS26a and VPS29-VPS35-VPS26b. See FIG. 19.

Some studies have suggested that the two paralogs might be functionally distinct (Bugarcic et al., 2011), while others suggest that they might be functionally redundant (Gallon et al., 2014, McMillan et al., 2016). A detailed comparison of the paralogs and their role of endosomal trafficking in neurons has never been reported.

Biomarkers for Neurodegenerative Diseases and Disorders where Retromer Dysfunction and/or Endosomal Trafficking Dysfunction is Involved and/or Implicated

Although the retromer-dependent endosomal trafficking pathway is strongly linked to AD, and the pathway can accelerate endosomal secretion, the identity of the proteins that accumulate in the cerebrospinal fluid (CSF) caused by impairing the pathway is unclear. Shown herein are the results using a genetically engineered mouse model in which Vps35 is knocked out selectively in forebrain neurons. VPS35 is the molecular backbone of retromer's heterotrimeric core, the scaffold to which all other retromer modules and retromer receptors bind (Small and Petsko, 2015). Numerous studies have established that, among other retromer-related proteins, depleting VPS35 is one of the most reliable ways to impair retromer-dependent endosomal traffic and recapitulate many of AD's core pathological features (as reviewed in (Small and Petsko, 2015)). Proteomic analysis was performed of CSF collected from the conditional Vps35 knockout (cKO) mice compared with CSF from control littermates, and then validated the three reliable findings with the greatest potential relevance to the goals of the study—the N terminus of two BACE1 substrates, APLP1 and CHL1, and the mid-domain of tau.

Guided by the mouse findings, assays which reliably measure the proteins in human CSF were developed and their relationships were investigated in a large-scale human study in patients with AD dementia, in patients with prodromal AD, and in healthy controls (HCs). In addition, because recent studies have suggested that elevations in tau phosphorylation at its threonine-217 site (p-tau217) represents one of the earliest and most specific CSF “signature” of AD (Barthelemy et al., 2020a; Barthelemy et al., 2020b), the relationship of APLP1 and CHL1 with p-tau217 was also investigated. Collectively, the results suggested that retromer-dependent endosomal trafficking can regulate CSF tau, APLP1, and CHL1, informing on how endosomal trafficking pathway in AD can contribute to disease spread and how to identify the trafficking impairments in vivo.

Additional validation studies were performed on n-APLP1 and n-CHL1 because, as the two are brain enriched, they are most suitable for human CSF biomarkers (Bergevoc et al., 2018). More than validating an increase in CSF n-APLP1 and n-CHL1, a relationship was observed between the two, in both controls and Vps35 cKO mice. In individual genotypes and collectively as a group, the relationship was nearly collinear across mice, with the Vps35 cKO mice simply shifting this relationship to the right. These relationships support the interpretation that when elevation in CSF n-APLP1 and n-CHL1 co-occur, they are likely reflective of retromer-dependent endosomal traffic dysfunction.

Fragments of the tau protein are also known to be found in the CSF (Cicognola et al. 2019; Meredith et al. 2013; Barthelemy et al. 2020a; Barthelemy et al. 2020b) and elevated in AD, but in this case, the mechanisms that regulate CSF tau accumulation have undergone a major shift in understanding. A number of mechanisms have been proposed for its secretion, but it is now clear that tau is not secreted by the conventional secretory pathway. As with the fragments of APP and its homologs, tau is also secreted via unconventional pathways. Besides translocation across the plasma membrane, a few previous studies have provided clues that tau might be actively secreted from the endosomal pathway. For example, tau is known to gain access to the endosomal pathway and retromer can influence intraneuronal tau pathology. Other studies have shown a tight linear correlation between CSF tau and amyloid in plaque-free human subjects, suggesting that they are secreted from the same pathway. The result herein corroborated this hypothesis by providing indications implicating the retromer-dependent endosomal pathway as one by which tau can be secreted from neurons.

By developing and validating a set of reagents that can assay n-APLP1 and n-CHL1 in human CSF with high precision, observations from the human CSF studies suggested that the conclusions drawn from the Vps35 cKO mice are true in humans. First, in a large-scale study in patients with mild to moderate AD dementia, a selective cross-correlation was found among n-APLP1, n-CHL1, and md-tau. Moreover, because this was a large-scale study, a stratification analysis was performed, of which the results supported the prediction that the existence of amyloid plaques, which are known to bind many extracellular peptides, influences the relationship among these three CSF proteins. This interpretation was further supported by the CSF study in HCs and patients with MCI. In controls, where a nearly collinear relationship among the three proteins was found, suggesting that in humans as in mice, all are secreted via the same unconventional pathway. In patients with MCI, a curvilinear relationship was observed between md-Tau and n-APLP1 or n-CHL1, and the relationship linearized when plaque correction was performed on the basis of CSF Aβ42 concentrations. By comparing the corrected n-APLP1 and n-CHL1 measures between HCs and patients with MCI, a CSF profile that phenocopies what was found when comparing Vps35 cKO mice and their control littermates was found: a tight linear relationship between n-APLP1 and n-CHL1 that were identical in slopes, but with a shift to the right in patients with MCI. Last, compared with HCs, patients who met the criteria for prodromal AD were found to have substantial, and concordant, elevations in CSF tau and plaque-corrected concentrations of n-APLP1 and n-CHL1.

The final analysis in human CSF further supports the interpretation that tau is, at least in part, actively released into the CSF via endosomal secretion. Recent reports have shown that compared with other CSF tau species, tau phosphorylation at its threonine-217 site (p-tau217) is the CSF tau signature that is most sensitive to AD, detected in the earliest preclinical stages of AD and before the onset of neurodegeneration, and the one most specific to AD compared with other tauopathies (Barthelemy et al. 2020a; Barthelemy et al. 2020b). Shown herein is that both n-APLP1 and n-CHL1 are associated with p-tau217 in all human CSF cohorts.

Additionally shown herein is the increased level of n-APLP1 in a mouse model of PD.

These findings support the use of biomarkers for impairments in retromer-dependent endosomal trafficking in AD and other neurodegenerative diseases and disorders. Because these proteins were identified in a mouse model of AD with impaired retromer function, these proteins can detect and identify AD and other neurodegenerative diseases and disorders where retromer dysfunction and/or endosomal trafficking dysfunction is involved and/or implicated.

Methods of Detecting Retromer Dysfunction in Subjects Having or Suspected of Having Neurodegenerative Disease or Disorder where Retromer Dysfunction and/or Endosomal Trafficking Dysfunction is Involved and/or Implicated

As stated above and shown in the Examples, certain protein markers are associated with the involvement of retromer dysfunction and/or endosomal in neurodegenerative diseases and disorders. These markers include but are not limited to n-APLP1 and n-CHL1 and tau.

By using these protein markers, important predictions and determinations can be made regarding the treatment of a patient's disease. While tests for these biomarkers can be performed at any time after a diagnosis of a neurodegenerative disease or disorder, preferably such tests would be performed as soon as possible after a positive diagnosis of neurodegenerative disease or disorder is made by a clinician, or as soon as a neurodegenerative disease or disorder is suspected. In that manner, the valuable insight into the disease can be utilized in choice of therapy.

In some embodiments, the subject may have symptoms of the neurodegenerative disease or disorder, including but not limited to MCI for AD and including but not limited to trembling of hands, arms, legs, jaw and face; stiffness of the arms, legs and trunk; slowness of movement; poor balance and coordination; and speech difficulty for PD.

The presence or amount of the protein markers can be compared to a reference value. In some embodiments, the reference value is from a healthy control. In some embodiments, the reference value is from a patient with a neurodegenerative disease or disorder where retromer dysfunction and/or endosomal trafficking dysfunction is involved and/or implicated. In some embodiments, the reference value is from the subject themselves at another time point in the disease or treatment.

In certain embodiments, a sample of biological tissue or bodily fluid from a subject with the neurodegenerative disease or disorder is obtained.

In certain embodiments, the sample is tested for protein levels of one or more of the markers including but not limited to n-APLP1 and n-CHL1 and tau. The protein sample can be obtained from any biological tissue. Preferred biological tissues include, but are not limited to, bone marrow, epidermal, whole blood, and plasma. The protein sample can be obtained from any biological fluid. Preferred fluids include, but are not limited to, cerebrospinal fluid (CSF), plasma, serum, saliva, and urine.

In some embodiments, the protein sample is from CSF.

Protein can be isolated and/or purified from the sample using any method known in the art, including but not limited to immunoaffinity chromatography.

While any method known in the art can be used, preferred methods for detecting and measuring increase levels of the proteins in a protein sample include flow cytometry, quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

Antibodies are a preferred method of detecting and measuring target or desired proteins in a sample. Such antibodies are available commercially or can be made by conventional methods known in the art. Such antibodies can be monoclonal or polyclonal and fragments thereof, and immunologic binding equivalents thereof. The term “antibody” means both a homologous molecular entity as well as a mixture, such as a serum product made up of several homologous molecular entities.

In a preferred embodiment, such antibodies will immunoprecipitate the desired proteins from a solution as well as react with desired/target proteins on a Western blot, immunoblot, ELISA, and other assays listed above.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents. A number of fluorescent materials are known and can be utilized as detectable labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. Any desired targets or binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. In embodiments the enzymes can be are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

In some embodiments, single-molecule-array (Simoa™) technology, a highly sensitive assay (Rissin et al. 2010), is used. Simoa™ testing is a powerful new technique that is orders of magnitude more sensitive than standard sandwich-based immunoassay techniques. Traditional ELISA measurements are limited to pg/ml levels of detection. Simoa™ can achieve sensitivity as low as femtogram (fg/ml) levels, allowing the detection and quantification of biomarkers at concentrations previously difficult or impossible to measure.

Simoa™ is based upon the isolation of individual immunocomplexes on paramagnetic beads using standard ELISA reagents. The main difference between Simoa™ and conventional immunoassays lies in the ability to trap single molecules in femtoliter-sized wells, allowing for a “digital” readout of each individual bead to determine if it is bound to the target analyte or not.

Described herein is the first use of this technology to detect protein biomarkers including tau, nAPLP1 and n-CHL1. Antibodies for use in the assay as well as determining the parameters for use of the technology to detect these proteins is described herein.

In some embodiments, the levels or amounts of n-APLP1 and n-CHL1 as well as tau found in a sample are compared to the levels or amounts of these proteins in a healthy control(s) and a deviation in the level or quantity of peptides is looked for. This comparison can be done in many ways. The same assay can be performed simultaneously or consecutively, on a purified and/or isolated protein sample from a healthy control(s) and the results compared qualitatively, e.g., visually, i.e., does the protein sample from the healthy control produce the same intensity of signal as the protein sample from the subject in the same assay, or the results can be compared quantitatively, e.g., a value of the signal for the protein sample from the subject is obtained and compared to a known reference value of the protein in a healthy control.

Alternatively, the levels or quantities of n-APLP1 and n-CHL1 as well as tau found in a sample are compared to the levels or amounts of these proteins in a subject with a neurodegenerative disease or disorder where retromer dysfunction and/or endosomal trafficking dysfunction is involved and/or implicated. In this embodiment, a similarity between the levels or amounts of protein is looked for. This comparison can also be done in many ways. The same assay can be performed simultaneously or consecutively, on a purified and/or isolated protein sample from a subject with a neurodegenerative disease or disorder where retromer dysfunction and/or endosomal trafficking dysfunction is involved and/or implicated and the results compared qualitatively, e.g., visually, i.e., does the protein sample from the subject with a neurodegenerative disease or disorder where retromer dysfunction and/or endosomal trafficking dysfunction is involved and/or implicated produce the same intensity of signal as the protein sample from the subject in the same assay, or the results can be compared quantitatively, e.g., a value of the signal for the protein sample from the subject is obtained and compared to a known reference value of the protein in a subject with a neurodegenerative disease or disorder where retromer dysfunction and/or endosomal trafficking dysfunction is involved and/or implicated.

Targeted Treatment for Neurodegenerative Diseases or Disorders where Retromer Dysfunction and/or Endosomal Trafficking Dysfunction is Involved and/or Implicated

Shown herein are studies that document at least in the brain, the two VPS26 paralogs are not interchangeable. Rather, in neurons each paralog is found to form a distinct trimeric retromer core, VPS26b-VPS35-VPS29 and VPS26a-VPS35-VPS29. Together with the observed expression profiles of the paralogs, this establishes that in contrast to other cells in our body, and phylogenetically down to yeast, neurons are endowed with a second retromer core. The studies herein show that, while some overlap might exist, the VPS26b core is dedicated more to endosomal recycling, a dominant trafficking pathway for maintaining synaptic health.

Showing that depleting a single endogenous protein, VPS26b, phenocopies AD's region vulnerability, together with VPS26b's dedicated role in glutamate-receptor recycling, mechanistically clarifies the disease's ‘anatomical biology’. Endosomal traffic dysfunction is now considered a central pathway in AD pathogenesis (Small et al., 2017), which can be affected by early-onset and late-onset genes. These results, together with the TEC's unique network properties (Small and Swanson, 2019), can generally explain why the TEC is vulnerable to all forms of the disease. Moreover, retromer-dependent endosomal traffic dysfunction has already been shown to act as an upstream event that can regulate its molecular biology—amyloid (Mecozzi et al., 2014) and tau pathology (Young et al., 2018, Li et al., 2019, Vagnozzi et al., 2019). By showing that, and suggesting how, endosomal traffic dysfunction can regulate Alzheimer's anatomical biology, these results strengthen trafficking's causal link to the disease and further validates trafficking as a therapeutic target.

Also shown herein is the rescue of a VPS26b KO mouse with a viral vector comprising VPS26b at the behavioral and electrophysiological level.

These results also show that a retromer therapeutic for AD would benefit by targeting the VPS26b retromer core, both to increase potential efficacy, and also, since this distinct core is brain enriched, to diminish potential side effects.

Additionally, retromer recycles cargo out of the endosome via two pathways. The first, whereby cargo is trafficked directly from the endosome back to the cell surface, would be the ‘direct’ pathway. The second, the “indirect” pathway, is where cargo is trafficked first to the trans-golgi network (TGN) and from there the cell surface.

Moreover, as shown in the Examples and discussed above, VPS26b is differentially expressed in cortical brain regions linked to Alzheimer's disease as well as differentially dedicated to the direct pathway, while VPS26a is differentially dedicated to the indirect pathway, and differentially expressed in subcortical brain regions linked to Parkinson's disease (PD).

Thus, VPS26a-retromer is the therapeutic target for PD.

Methods of Treating, Preventing, and/or Curing Neurodegenerative Diseases or Disorders where Retromer Dysfunction and/or Endosomal Trafficking Dysfunction is Involved and/or Implicated Patients who would benefit from the methods described herein include those diagnosed with, suspected of having, or at risk for a neurogenerative disease or disorder where retromer dysfunction and/or endosomal trafficking defects are implicated including but not limited to Alzheimer's disease (AD), Parkinson's disease (PD), Amyotrophic Lateral Sclerosis (ALS), Frontal-Temporal Degeneration, Downs Syndrome, and Prion Disease. As disclosed herein, once a patient has been identified as having a neurodegenerative disease or disorder where retromer dysfunction and/or endosomal trafficking defects are implicated, then the appropriate retromer therapeutic can be chosen and administered.

In some embodiments, the retromer therapeutic is a composition containing a nucleic acid or a transgene encoding one or more of the retromer proteins described herein. The composition may be a vector, for example, a viral vector, such as an adeno-associated virus (AAV) vector.

In some embodiments, the retromer therapeutic is a composition or compositions containing one or more pharmacological retromer chaperones.

In some embodiments, the retromer therapeutic is both a vector, for example, a viral vector, such as an adeno-associated virus (AAV) vector and one or more pharmacological retromer chaperones.

In some embodiments, the neurodegenerative disease or disorder is Alzheimer's disease (AD). As described herein, subjects with Alzheimer's disease would benefit most from the administration of a retromer therapeutic which is a composition containing a nucleic acid or a transgene encoding VPS35 alone or VPS26b alone, and/or a pharmacological chaperone that increases or stabilizes VPS35 and/or VPS26b.

In some embodiments, the neurodegenerative disease or disorder is Parkinson's disease (AD). As described herein, subjects with Parkinson's disease would benefit most from the administration of a retromer therapeutic which is a composition containing a nucleic acid or a transgene encoding VPS35 alone or VPS26a alone, and/or a pharmacological chaperone that increases or stabilizes VPS35 and/or VPS26a.

In some embodiments, only one composition, e.g., a composition containing a nucleic acid encoding one of the retromer core proteins (e.g., viral vectors, such as AAV vectors, containing such nucleic acids) is administered to the subject. In some embodiments, a second composition, e.g., a composition comprising one or more pharmacological chaperones. A first composition containing a nucleic acid encoding one or more of the retromer core proteins (e.g., viral vectors, such as AAV vectors, containing such nucleic acids) can also be administered to the patient in combination with a second composition or compositions comprising one or more pharmacological retromer chaperones. These compositions may be administered alone or in further combination with other agents for the treatment of neurodegenerative diseases or disorders.

In some embodiments, the first composition and the second composition are administered to the subject simultaneously.

In some embodiments, the first composition and the second composition are administered to the subject sequentially.

In some embodiments, the second composition is administered to the subject after administration of the first composition to the subject. The second composition may be administered to the subject, for example, within one or more days or weeks of administration of the first composition to the subject. In some embodiments, the second composition is administered to the subject at least one month after administration of the first composition to the subject. In some embodiments, administration of the first composition continues while the second composition is administered to the subject.

In some embodiments, the first composition is administered to the subject after administration of the second composition to the subject. The first composition may be administered to the subject, for example, within one or more days or weeks of administration of the second composition to the subject. In some embodiments, the first composition is administered to the subject at least one month after administration of the second composition to the subject. In some embodiments, administration of the second composition continues while the first composition is administered to the subject.

In some embodiments, the first composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the second composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the composition or compositions (e.g., viral vector, such as an AAV) comprising a nucleic acid encoding retromer core protein VPS35 or retromer core protein VPS26a or retromer core protein VPS26b and a composition or compositions comprising one or more pharmacological retromer chaperones is administered as soon as neurodegenerative disease or disorder is diagnosed or suspected.

In some embodiments, the amount of AAV vector comprising the transgene administered is about 4.2×10¹¹ or 4.2×10¹⁰ genome or vector or vector copies. It is expected that a lower amount of viral vector could be administered when it is administered in conjunction with one or more pharmacological chaperones.

In some embodiments, the co-administration of gene therapy and pharmacological retromer chaperones, at least additive effects can be obtained as compared to the use of either single active agent alone, i.e., without the other, and lower dosages of each component can be administered to the subject than if either was being administered alone.

In one embodiment of the present disclosure, the first composition is administered via injection into the brain and the second composition is administered orally.

“Recombinant AAV (rAAV) vectors” described herein generally include a transgene (e.g., encoding retromer core protein VPS35 or retromer core protein VPS26a or retromer core protein VPS26b). The transgene is flanked by 5′ and 3′ ITRs and may be operably linked to one or more regulatory elements in a manner that permits transgene transcription, translation, and/or expression in a cell of a target tissue. Such regulatory elements may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). The AAV vector may then be delivered to a selected target cell type or tissue. In some embodiments, the transgene is a nucleic acid sequence, heterologous to the vector sequences, which encodes one or more of VPS35, VPS26a or VPS26b. Components of exemplary AAV vectors that may be used in conjunction with the compositions and methods of the disclosure are described herein.

Any AAV serotype or combination of AAV serotype can be used in the methods and compositions of the present disclosure. Because the methods and compositions of the present disclosure are for the treatment and cure of neurodegenerative diseases or disorders, AAV serotypes that target at least the central nervous system can be used in some embodiments and include but are not limited to AAV1, AAV2, AAV4, AAV5, AAV6, AAV7, AAV8, AAV9, and AAV10.

In some embodiments, AAV9 serotype, which has a wide tropism, is used. In some embodiments, an AAV2/9 is used.

Components of AAV Vectors

The AAV vectors described herein may contain cis-acting 5′ and 3′ ITRs (See, e.g., Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, (1989) and Fisher et al., (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins. In some embodiments, operably linked coding sequences yield a fusion protein. In some embodiments, operably linked coding sequences yield a functional RNA (e.g., shRNA, miRNA). In some embodiments, operably linked coding sequences yield two or more separate functional proteins.

For nucleic acids encoding proteins, a polyadenylation sequence generally is inserted following the transgene sequences and before the 3′ AAV ITR sequence. An rAAV construct of the present disclosure may also contain an intron, desirably located between the promoter/enhancer sequence and the transgene. One possible intron sequence is derived from SV-40 and is referred to as the SV-40 T intron sequence.

Another vector element that may be used is an internal ribosome entry site (IRES). An IRES sequence is used to produce more than one polypeptide or protein from a single transcript.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a β-actin promoter, a phosphoglycerol kinase (PGK) promoter, and a human elongation factor-1a (EF1a) promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system (WO 98/10088); a ecdysone insect promoter (No et al., Proc. Natl. Acad. Sci. USA 93:3346-3351 (1996)), a tetracycline-repressible system (Gossen et al., Proc. Natl. Acad. Sci. USA 89:5547-5551 (1992)), a tetracycline-inducible system (Gossen et al., Science 268:1766-1769 (1995), a RU486-inducible system (Wang et al., Nat. Biotech. 15:239-243 (1997) and Wang et al., Gene Ther. 4:432-441 (1997)) and a rapamycin-inducible system (Magari et al., J. Clin. Invest. 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, a native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner.

In some embodiments, one or more bindings sites for one or more of miRNAs are incorporated in a transgene of a rAAV vector, to inhibit the expression of the transgene in one or more tissues of a subject harboring the transgenes. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-or more nucleotides. The target site sequence may comprise at least 5 nucleotides of the sequence of a target gene binding site.

For example, a 3′ UTR site which would inhibit the expression of the transgene in the liver can be incorporated into a transgene. This would be beneficial for transgenes which encode therapeutic proteins which are toxic to the liver as most of the virus administered (approximately 60 to 90%) is eventually found in the liver. Thus suppressing the therapeutic gene expression in liver relieves the burden from liver cells.

In some embodiments, the AAV vector will be modified to be a self-complementing AAV. A self-complementing AAV carries complementary sequence of the transgene (i.e., a double copy of the transgene). Self-complementation makes the gene more stable after it enters the cell.

Transgene Coding Sequences

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In embodiments. the transgene encodes a functional protein including but not limited to retromer core protein VPS35 or retromer core protein VPS26a or retromer core protein VPS26b.

It is noted that as used herein VPS35, VPS26a and VPS26b can refer to the gene or the protein encoded for by the gene, as appropriate in the specific context utilized. Additionally, in certain contexts, the reference will be to the mouse gene or protein, and in others the human gene or protein as appropriate in the specific context.

The amino acid sequence information can be obtained from the National Center for Biotechnology Information (NCBI) and are set forth below.

The gene encoding the human retromer core protein VPS35 (Gene ID: 55737) can be used to obtain a transgene encoding a functional retromer core protein VPS35.

In some embodiments, the transgene encodes retromer core protein VPS35. The retromer core protein VPS35 encoded by the transgene may have an amino acid sequence that is at least 85% identical to the amino acid sequence of VPS35 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 90% identical to the amino acid sequence of VPS35 (e.g., an amino acid sequence that is 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 95% identical to the amino acid sequence of VPS35 (e.g., an amino acid sequence that is 95%, 96%, 97%, 98%, 99%, or 100% identical to the amino acid sequence of VPS35).

In some embodiments, the retromer core protein VPS35 encoded by the transgene has an amino acid sequence that differs from VPS35 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS35 encoded by the transgene has an amino acid sequence that differs from VPS35 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 16, 17, 18, 19, 20, 21, 22, 23, 24, 25, or more, conservative amino acid substitutions).

In some embodiments, the transgene encoding retromer core protein VPS35 has a nucleic acid sequence that is at least 70% identical to the nucleic acid sequence encoding VPS35 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 85% identical to the nucleic acid sequence encoding VPS35 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 90% identical to the nucleic acid sequence encoding VPS35 (e.g., a nucleic acid sequence that is 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 95% identical to the nucleic acid sequence encoding VPS35 (e.g., a nucleic acid sequence that is 95%, 96%, 97%, 98%, 99%, or 100% identical to the nucleic acid sequence encoding VPS35).

In some embodiments, the transgene encodes the human retromer core protein VPS35 comprising SEQ ID NO: 1. The retromer core protein VPS35 encoded by the transgene may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the retromer core protein VPS35 encoded by the transgene has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the retromer core protein VPS35 encoded by the transgene has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1).

In some embodiments, the retromer core protein VPS35 encoded by the transgene has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS35 encoded by the transgene has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 16, 17, 18, 19, 20, 21, 22, 23, 24, 25, or more, conservative amino acid substitutions).

In some embodiments, the transgene encoding retromer core protein VPS35 has a nucleic acid sequence that is at least 70% identical to the nucleic acid sequence encoding SEQ ID NO: 1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 85% identical to the nucleic acid sequence encoding SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 90% identical to the nucleic acid sequence encoding SEQ ID NO: 1 (e.g., a nucleic acid sequence that is 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 95% identical to the nucleic acid sequence encoding SEQ ID NO: 1 (e.g., a nucleic acid sequence that is 95%, 96%, 97%, 98%, 99%, or 100% identical to the nucleic acid sequence encoding SEQ ID NO: 1).

In some embodiments, the transgene encoding VPS35 comprises SEQ ID NO: 2.

In some embodiments, the transgene encoding retromer core protein VPS35 is codon optimized.

In some embodiments, the transgene encoding retromer core protein VPS35 comprise SEQ ID NO: 3.

In some embodiments, the transgene encoding retromer core protein VSP35 is a modified human VPS35 coding sequence (certain codons modified to remove restriction sites) and comprises SEQ ID NO: 10.

The gene encoding the human retromer core protein VPS26a (Gene ID: 9559) can be used to obtain a transgene encoding a functional retromer core protein VPS26a.

The retromer core protein VPS26a encoded by the transgene may have an amino acid sequence that is at least 85% identical to the amino acid sequence of VPS26a (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 90% identical to the amino acid sequence of VPS26a (e.g., an amino acid sequence that is 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 95% identical to the amino acid sequence of VPS26a (e.g., an amino acid sequence that is 95%, 96%, 97%, 98%, 99%, or 100% identical to the amino acid sequence of VPS26a).

In some embodiments, the retromer core protein VPS26a encoded by the transgene has an amino acid sequence that differs from VPS26a by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein Vps26a has an amino acid sequence that differs from VPS26a by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26a has a nucleic acid sequence that is at least 70% identical to the nucleic acid sequence encoding VPS26a (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26a). In some embodiments, the transgene encoding retromer core protein VPS26a has a nucleic acid sequence that is at least 85% identical to the nucleic acid sequence encoding VPS26a (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding VPS26a). In some embodiments, the transgene encoding retromer core protein VPS26a has a nucleic acid sequence that is at least 90% identical to the nucleic acid sequence encoding VPS26a (e.g., a nucleic acid sequence that is 90%, 91%, 92%, 93%, 94%, 95%, 96%, 97%, 98%, 99%, or 100% identical to the nucleic acid sequence of encoding VPS26a). In some embodiments, the transgene encoding retromer core protein Vps26a has a nucleic acid sequence that is at least 95% identical to the nucleic acid sequence encoding VPS26a (e.g., a nucleic acid sequence that is 95%, 96%, 97%, 98%, 99%, or 100% identical to the nucleic acid sequence of encoding VPS26a).

In some embodiments, the transgene encodes the human retromer core protein VPS26a comprising SEQ ID NO: 4. The retromer core protein VPS26a encoded by the transgene may have an amino acid sequence that is at least 85% identical to the amino acid sequence of SEQ ID NO: 4 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 4). In some embodiments, the retromer core protein VPS26a encoded by the transgene has an amino acid sequence that is at least 90% identical to the amino acid sequence of SEQ ID NO: 4 (e.g., an amino acid sequence that is 90%, 91%, 92%, 93%, 94%, 95%, 96%, 97%, 98%, 99%, or 100% identical to the amino acid sequence of SEQ ID NO: 4). In some embodiments, the retromer core protein VPS26a encoded by the transgene has an amino acid sequence that is at least 95% identical to the amino acid sequence of SEQ ID NO: 4 (e.g., an amino acid sequence that is 95%, 96%, 97%, 98%, 99%, or 100% identical to the amino acid sequence of SEQ ID NO: 4).

In some embodiments, the retromer core protein VPS26a encoded by the transgene has an amino acid sequence that differs from SEQ ID NO: 4 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS6a has an amino acid sequence that differs from SEQ ID NO: 4 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26a has a nucleic acid sequence that is at least 70% identical to the nucleic acid sequence encoding SEQ ID NO: 4 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 4). In some embodiments, the transgene encoding retromer core protein VPS26a has a nucleic acid sequence that is at least 85% identical to the nucleic acid sequence encoding SEQ ID NO: 4 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding SEQ ID NO: 4). In some embodiments, the transgene encoding retromer core protein VPS26a has a nucleic acid sequence that is at least 90% identical to the nucleic acid sequence encoding SEQ ID NO: 4 (e.g., a nucleic acid sequence that is 90%, 91%, 92%, 93%, 94%, 95%, 96%, 97%, 98%, 99%, or 100% identical to the nucleic acid sequence of encoding SEQ ID NO: 4). In some embodiments, the transgene encoding retromer core protein VPS26a has a nucleic acid sequence that is at least 95% identical to the nucleic acid sequence encoding SEQ ID NO: 4 (e.g., a nucleic acid sequence that is 95%, 96%, 97%, 98%, 99%, or 100% identical to the nucleic acid sequence of encoding SEQ ID NO: 4).

In some embodiments, the transgene encoding VPS26a comprises SEQ ID NO: 5.

In some embodiments, the transgene encoding retromer core protein VPS26a is codon optimized.

In some embodiments, the transgene encoding retromer core protein VPS26a comprises SEQ ID NO: 6.

The gene encoding the human retromer core protein VPS26b (Gene ID: 112936) can be used to obtain a transgene encoding a functional retromer core protein

In some embodiments, the transgene encodes retromer core protein VPS26b. The retromer core protein VPS26b encoded by the transgene may have an amino acid sequence that is at least 85% identical to the amino acid sequence of VPS26b (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 90% identical to the amino acid sequence of VPS26b (e.g., an amino acid sequence that is 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 95% identical to the amino acid sequence of VPS26b (e.g., an amino acid sequence that is 95%, 96%, 97%, 98%, 99%, or 100% identical to the amino acid sequence of VPS26b).

In some embodiments, the retromer core protein VPS26b encoded by the transgene has an amino acid sequence that differs from VPS26b by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS26b encoded by the transgene has an amino acid sequence that differs from VPS26b by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 16, 17, 18, 19, 20, 21, 22, 23, 24, 25, or more, conservative amino acid substitutions).

In some embodiments, the transgene encoding retromer core protein VPS26b has a nucleic acid sequence that is at least 70% identical to the nucleic acid sequence encoding VPS26b (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26b). In some embodiments, the transgene encoding retromer core protein VPS26b has a nucleic acid sequence that is at least 85% identical to the nucleic acid sequence encoding VPS26b (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding VPS26b). In some embodiments, the transgene encoding retromer core protein VPS26b has a nucleic acid sequence that is at least 90% identical to the nucleic acid sequence encoding VPS26b (e.g., a nucleic acid sequence that is 90%, 91%, 92%, 93%, 94%, 95%, 96%, 97%, 98%, 99%, or 100% identical to the nucleic acid sequence of encoding VPS26b). In some embodiments, the transgene encoding retromer core protein Vps26b has a nucleic acid sequence that is at least 95% identical to the nucleic acid sequence encoding VPS26b (e.g., a nucleic acid sequence that is 95%, 96%, 97%, 98%, 99%, or 100% identical to the nucleic acid sequence of encoding VPS26b).

In some embodiments, the transgene encodes the human retromer core protein VPS26b comprising SEQ ID NO: 7. The retromer core protein VPS26b encoded by the transgene may have an amino acid sequence that is at least 85% identical to the amino acid sequence of SEQ ID NO: 7 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 7). In some embodiments, the retromer core protein VPS26b encoded by the transgene has an amino acid sequence that is at least 90% identical to the amino acid sequence of SEQ ID NO: 7 (e.g., an amino acid sequence that is 90%, 91%, 92%, 93%, 94%, 95%, 96%, 97%, 98%, 99%, or 100% identical to the amino acid sequence of SEQ ID NO: 7). In some embodiments, the retromer core protein VPS26b encoded by the transgene has an amino acid sequence that is at least 95% identical to the amino acid sequence of SEQ ID NO: 7 (e.g., an amino acid sequence that is 95%, 96%, 97%, 98%, 99%, or 100% identical to the amino acid sequence of SEQ ID NO: 7).

In some embodiments, the retromer core protein VPS26b encoded by the transgene has an amino acid sequence that differs from SEQ ID NO: 7 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS6b has an amino acid sequence that differs from SEQ ID NO: 7 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26b has a nucleic acid sequence that is at least 70% identical to the nucleic acid sequence encoding SEQ ID NO: 7 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 7). In some embodiments, the transgene encoding retromer core protein VPS26b has a nucleic acid sequence that is at least 85% identical to the nucleic acid sequence encoding SEQ ID NO: 7 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding SEQ ID NO: 7). In some embodiments, the transgene encoding retromer core protein VPS26b has a nucleic acid sequence that is at least 90% identical to the nucleic acid sequence encoding SEQ ID NO: 7 (e.g., a nucleic acid sequence that is 90%, 91%, 92%, 93%, 94%, 95%, 96%, 97%, 98%, 99%, or 100% identical to the nucleic acid sequence of encoding SEQ ID NO: 7). In some embodiments, the transgene encoding retromer core protein VPS26b has a nucleic acid sequence that is at least 95% identical to the nucleic acid sequence encoding SEQ ID NO: 7 (e.g., a nucleic acid sequence that is 95%, 96%, 97%, 98%, 99%, or 100% identical to the nucleic acid sequence of encoding SEQ ID NO: 4).

In some embodiments, the transgene encoding VPS26b comprises SEQ ID NO: 8.

In some embodiments, the transgene encoding retromer core protein VPS26b is codon optimized.

In some embodiments, the transgene encoding retromer core protein VPS26b comprises SEQ ID NO: 9.

Codon Optimization of Transgene Coding Sequences

Codon optimization of the transgene coding sequences can increase the efficiency of the gene therapy. Thus, in some embodiments, a nucleic acid that is at least 70% identical to the coding sequence of the transgene encoding the therapeutic protein (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence) is used.

Codon optimization tools are known in the art.

Exemplary codon optimized nucleic acids include SEQ ID NOs: 3, 6, and 9.

Routes of Administration and Dosing for rAAV Vectors

The current disclosure provides rAAV vectors for use in methods of treating, preventing, and/or curing a neurodegenerative disease or disorder and/or alleviating in a subject at least one of the symptoms associated with a neurodegenerative disease and/or disorder. In some embodiments, methods involve administration of a rAAV vector that encodes one or more therapeutic polypeptides or proteins, in a pharmaceutically-acceptable carrier to the subject in an amount and for a period of time sufficient to treat, prevent and/or cure the neurodegenerative disease or disorder in the subject having or suspected of having such a neurodegenerative disease or disorder.

The rAAV vectors may be delivered to a subject in compositions according to any appropriate methods known in the art. The rAAV vector, preferably suspended in a physiologically compatible carrier (e.g., in a composition), may be administered to a subject. In one embodiment, a composition can comprise an rAAV9 vector comprising a nucleic acid sequence comprising a transgene encoding a functional protein including but not limited to retromer core protein VPS35 or retromer core protein VPS26a or retromer core protein VPS26b. In one embodiment, a composition can comprise an rAAV2/9 vector comprising a nucleic acid sequence comprising a transgene encoding a functional protein including but not limited to retromer core protein VPS35 or retromer core protein VPS26a or retromer core protein VPS26b. In one embodiment, a composition can comprise an rAAV10 or rAAV2/10 vector comprising a nucleic acid sequence comprising a transgene encoding a functional protein including but not limited to retromer core protein VPS35 or retromer core protein VPS26a or retromer core protein VPS26b.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions disclosed herein may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pH adjustment, and salt concentration adjustment (see, e.g., Wright, et al., Molecular Therapy 12:171-178 (2005).

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

rAAVS are administered by a route of administration and in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, and other parental routes of administration. Routes of administration may be combined, if desired. The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

The present disclosure provides stable pharmaceutical compositions comprising rAAV virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art. In some embodiments, the effective dose will be less for the rAAV administered with the pharmacological retromer chaperone than if administered alone.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration; the level of gene or RNA expression required to achieve a therapeutic effect; the specific disease or disorder being treated; and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target desired tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby. Effective dosages can be readily established by one of ordinary skill in the art through routine trials establishing dose response curves. A “therapeutically effective amount” will be less for the rAAV administered with the pharmacological retromer chaperone than if administered alone.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, three, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient or carriers. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions.

Toxicity and therapeutic efficacy of the therapeutic compositions, administered alone or in combination with another agent, can be determined by standard pharmaceutical procedures in cell cultures or experimental animals, e.g., for determining the LD⁵⁰ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. The dose may begin with an amount somewhat less than the optimum dose and it may be increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

A preferred route of administration of the AAVs is intravenously. Other routes of administration of the rAAV vectors described herein include intracranial, intraparenchymal, intraspinal.

A preferred dose ranges from about 1×10¹⁰ to about 8×10¹¹, from about 2×10¹⁰ to about 6×10¹¹, from about 4×10¹⁰ to about 4×10¹¹ genome or viral copy (vc) total administration. A preferred dose is about 4×10¹¹ genome or viral copy (vc) total administration of rAAV.

If more than one rAAV is used a preferred total dose of vector ranges from about 1×10¹⁰ to about 6×10¹¹, from about 2×10¹⁰ to about 5×10¹¹, from about 1×10¹⁰ to about 4×10¹¹ genome or viral copy (vc) total administration. A preferred dose of total vector is about 3×10¹¹. The AAV can be administered in equal amounts, e.g., ratio of 50/50, or in or in ratios of about 5/95, 15/85, 20/80, 25/75, 30/70, 35/65, 40/60, 45/55, 55/45, 60/40, 65/35, 70/30, 75/25, 85/15, 90/10, and 95/5.

Doses can be adjusted to optimize the effects in the subject. Additionally, a subject can be monitored for improvement of their condition prior to increasing the dosage. A subject's response to the therapeutic administration of the rAAV can be monitored by observing a subject's muscle strength and control, and mobility as well as changes in height and weight. If one or more of these parameters increase after the administration, the treatment can be continued. If one or more of these parameters stays the same or decreases, the dosage can be increased.

Again dosages can be adjusted and the therapeutically effective amount or dosage less when the viral vectors are administered in conjunction with the pharmacological chaperones.

### Pharmacological Retromer Chaperones

As stated above, a pharmacological retromer chaperone is a small molecule or other agent that binds to the retromer protein, and by virtue of stabilizing the protein's three-dimensional structure, protects it from degradation and increases its steady-state concentration in the cell. See Mecozzi et al. 2014, herein incorporated in its entirety.

Using crystal structures of the retromer protein, putative binding sites were located and a large-scale in silico screen performed to identify small molecules that would act as retromer chaperones. Twenty-four of the top 100 predicted binding compounds were incubated with purified, reconstituted heterotrimeric complex at various concentrations and the denaturation temperature of the complex measured. One compound designated R55 improved thermal stability significantly. This compound increased VPS35 and VPS26 protein levels in cultured primary neurons. Further experiments showed it to be a retromer chaperone. See Mecozzi et al. 2014, herein incorporated in its entirety.

Thus in one embodiment of the present disclosure the pharmacological retromer chaperone is R55. R55, is a thiophene thiourea derivative with molecule weight 260.00 in free base form. It is also known as TPT-260. R55 binds to a characterized hot spot at the interface between VPS35 and VPS29. R55 has the chemical structure below.

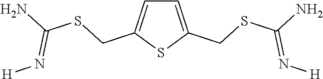 [SMILES: [H]/N=C(\N)SCc1ccc(CS/C(N)=N/[H])s1]

In a further embodiment, the pharmacological retromer chaperone is R33. R33 is also thiophene thiourea derivative and has a molecule weight 172 in free base form. It is also known as TPT-172. R33 has the chemical structure below.

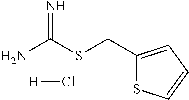

As described R55 and R33 bind at the interface of VPS35 and VPS29. Thus, other pharmacological retromer chaperones that have a structure that can bind to this interface can also be used in the disclosed methods and compositions. Using the techniques and results set forth in Mecozzi and structure activity relationships (SAR), one of skill in the art could determine additional pharmacological retromer chaperones that can be used in the compositions and methods disclosed herein.

For example, the 2,5-disubstituted thiophene scaffold in R55 can be replaced with a phenyl ring as they are bio-isosters. Additionally, guanylhydrazones can be substituted for isothioureas, as the former has good stability and a pKa ranger closer to neutrality. Pharmacological retromer chaperones containing one or both of these substitutions to R55 can be used in the methods and compositions described herein including the compound set forth below. See for example Muzio et al. 2020.

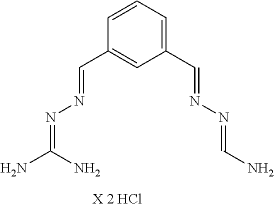 [SMILES: N/C=N/N=C/c1cccc(/C=N/N=C(N)N)c1]

In a further embodiment, the pharmacological retromer chaperone is a small molecule or another agent which binds to the retromer at site 2, the largest potential ligand-binding site at the interface of VPS29 and VPS35.

In a further embodiment, the pharmacological retromer chaperone is a small molecule or agent which binds to the retromer and increases its stability.

It will be understood by those of skill in the art that additional pharmacological retromer chaperones can be identified or synthesized using the structure of the retromer complex.

All of the pharmacological retromer chaperones can be in the form of pharmaceutical compositions.

Most preferred methods of administration of the compositions containing the pharmacological retromer chaperones for use in the methods disclosed herein are oral, intrathecal, nasal, and parental including intravenous, with oral administration being preferred. The pharmacological agent must be in the appropriate form for administration of choice.

Such pharmaceutical compositions comprising one or more pharmacological retromer chaperones for administration may comprise a therapeutically effective amount of the pharmacological retromer chaperones and a pharmaceutically acceptable carrier.

The phrase “pharmaceutically acceptable” as used herein refers to molecular entities and compositions that are physiologically tolerable and do not typically produce an allergic or similar untoward reaction, such as gastric upset, dizziness and the like, when administered to a human,

Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

In order to overcome any issue of the pharmacological agents crossing the blood/brain barrier, intrathecal administration is a further preferred form of administration. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Further methods of administration include sublingual, vaginal, buccal, or rectal; or transdermal administration to a subject.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

Doses can be adjusted to optimize the effects in the subject. For example, the pharmacological retromer chaperone can be administered at a low dose to start and then increased over time to depending upon the subject's response. A subject can be monitored for improvement of their condition prior to changing, i.e., increasing or decreasing, the dosage. A subject can also be monitored for adverse effects prior to changing the dosage, i.e., increasing or decreasing, the dosage.

Effective dosages can be readily established by one of ordinary skill in the art through routine trials establishing dose response curves. A “therapeutically effective amount” will be less for the pharmacological retromer chaperone when administered with the gene therapy, e.g., viral vector comprising a transgene encoding a retromer core protein, than if administered alone.

Dosage treatment may be a single dose schedule or a multiple dose schedule to ultimately deliver the amount specified above. Moreover, the subject may be administered as many doses as appropriate. Thus, the subject may be given, a single dose, or two, three, four, five, six or more doses that collectively result in delivery of a therapeutically effective dose. One of skill in the art can readily determine an appropriate number of doses to administer.

In some embodiments, the dose is about 75 mg/kg of pharmacological retromer chaperone.

### Kits

The present disclosure also provides kits comprising the components of the combinations disclosed herein in kit form. A kit of the present disclosure includes one or more components including, but not limited to, viral vectors (e.g., AAV vectors) and compositions comprising pharmacological retromer chaperones described herein. Kits may further include a pharmaceutically acceptable carrier, as discussed herein. The viral vector can be formulated as a pure composition or in combination with a pharmaceutically acceptable carrier, in a pharmaceutical composition.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-8

### Study Design

An unbiased screen of neuronal proteins that accumulate in murine CSF in the setting of endosomal dysfunction using a mouse model deficient for the retromer core protein, VPS35, was performed. The experimenter was blinded in all mouse and human studies. Male and female mice used in this study were randomly selected for the respective group. Determination of sample sizes was based on previous experiences with mouse studies. Approximately 40 mice per genotype were used for the mass spectrometry experiments. CSF was collected in a post-mortem procedure from 6 month-old Vps35 cKO males and littermate controls. Following blood contamination assessment via hemoglobin ELISA, contamination-free samples from a total of 52 mice were pooled into biological replicates of 30 uL each (n=4-7 per genotype) for proteomic LC-MS/MS analysis. It was confirmed that using this approach, the technical replicates were highly correlated as were the mean scores for biological replicates of the same genotype, indicating robustness of technique. Outliers were identified via Spearman correlation of technical replicates and one sample from each genotype was removed prior to subsequent analysis. Further validation of the proteomic hits was performed by Western blot and Simoa™ analysis using two additional cohorts of mice at 3 and 6 months of age (the n for each experimental group is specified in figure legend).

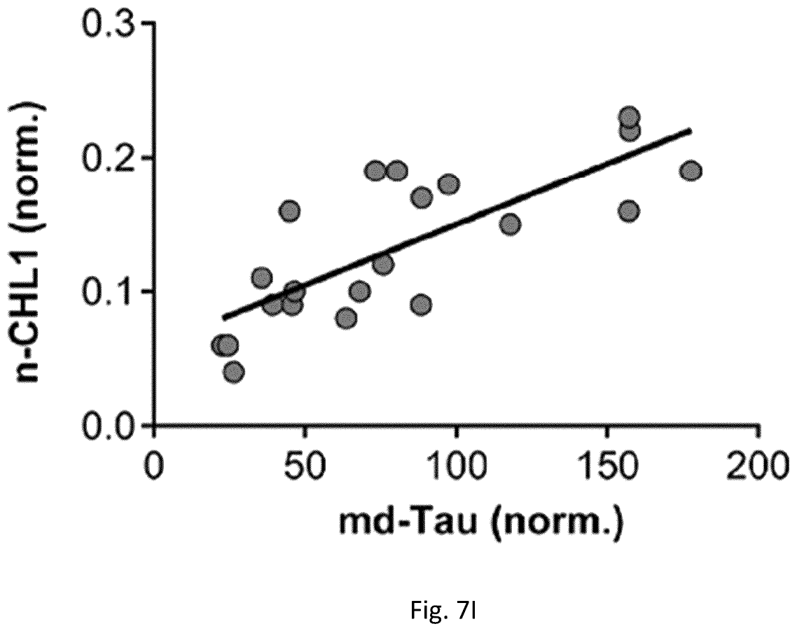

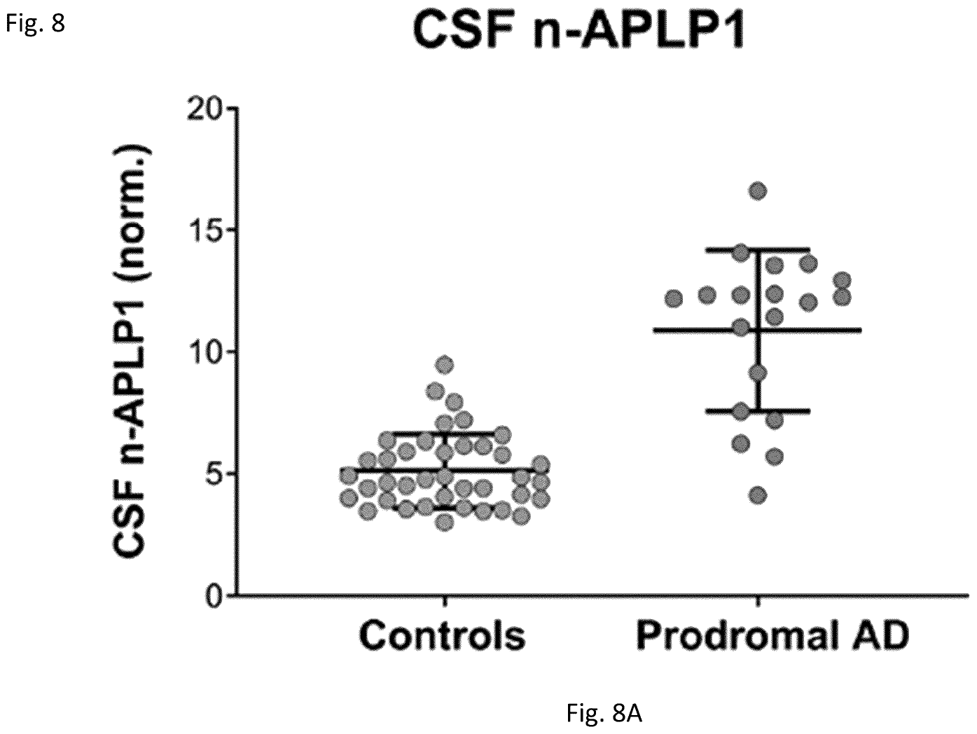

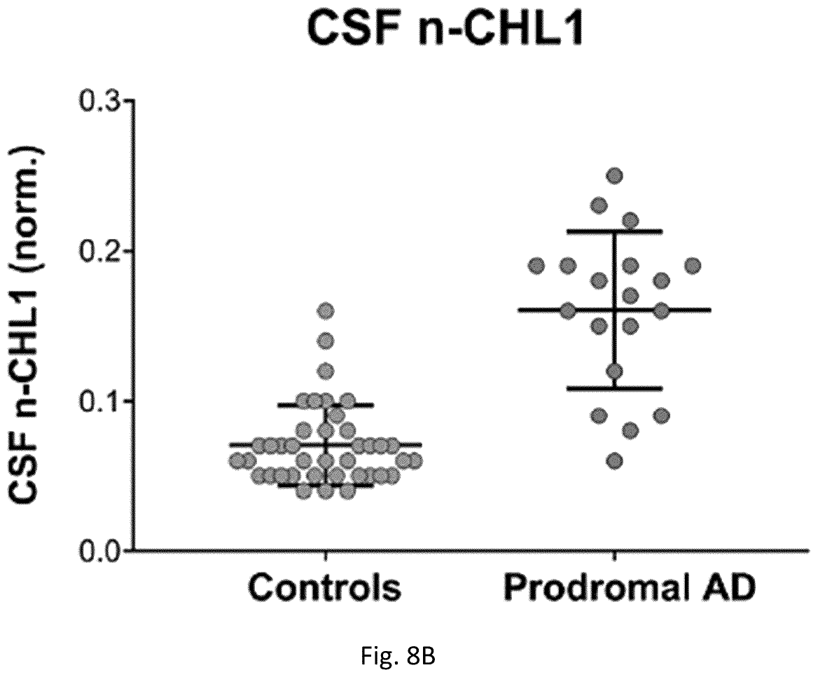

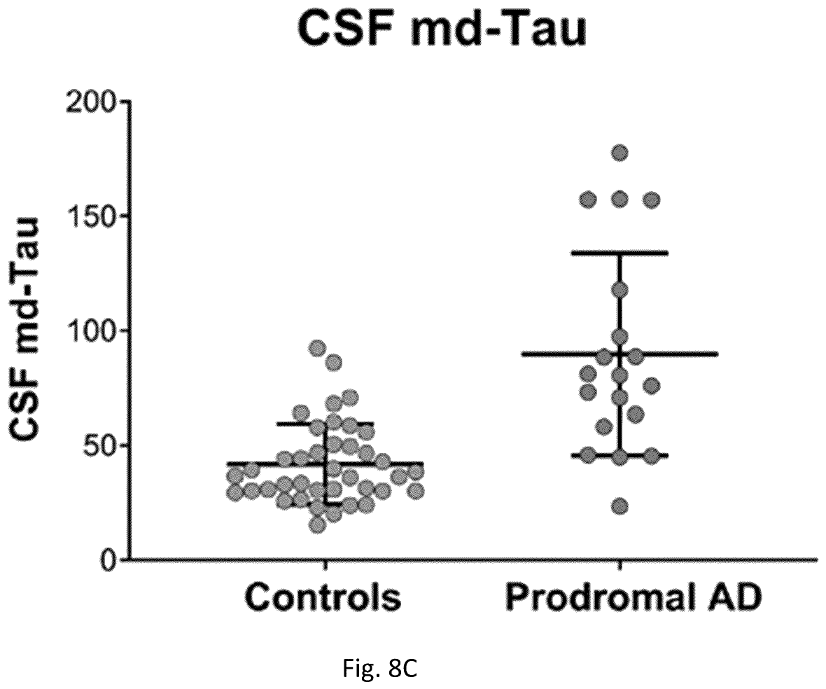

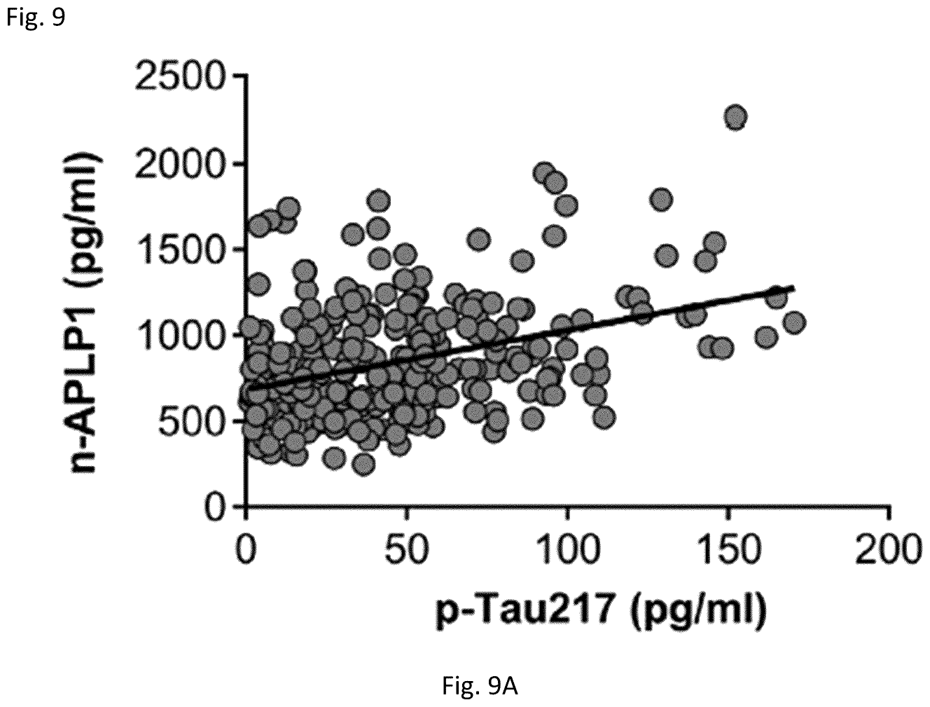

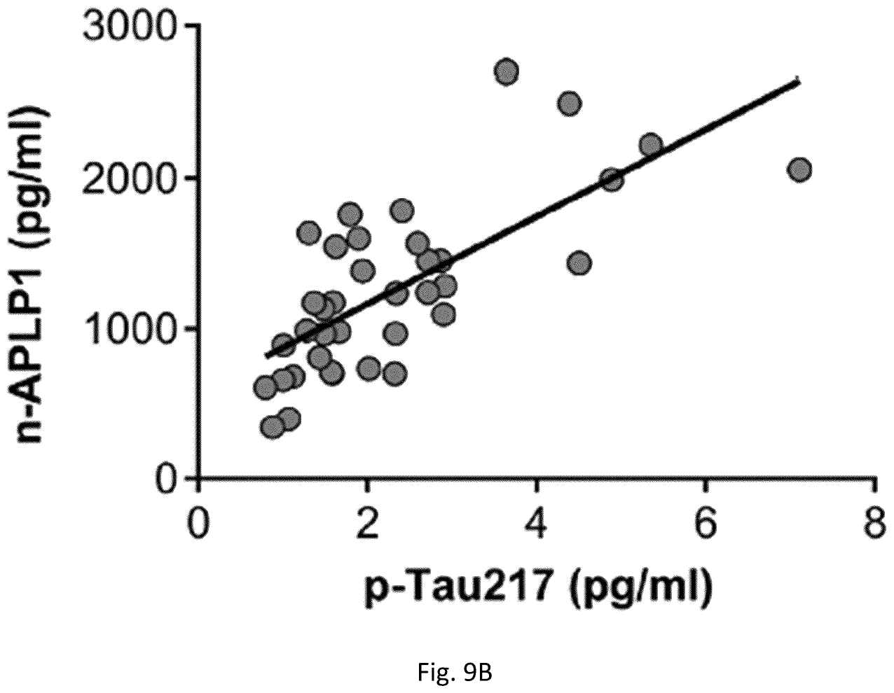

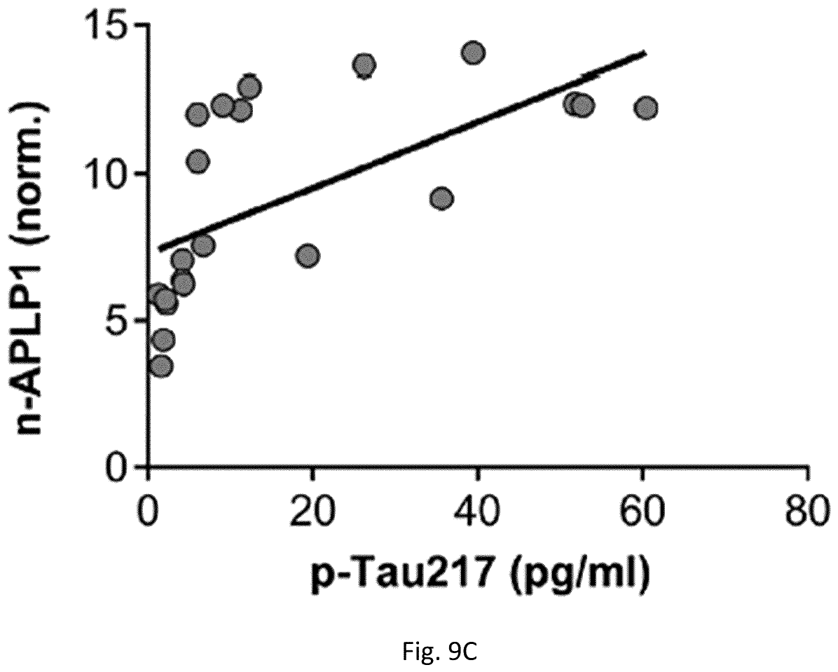

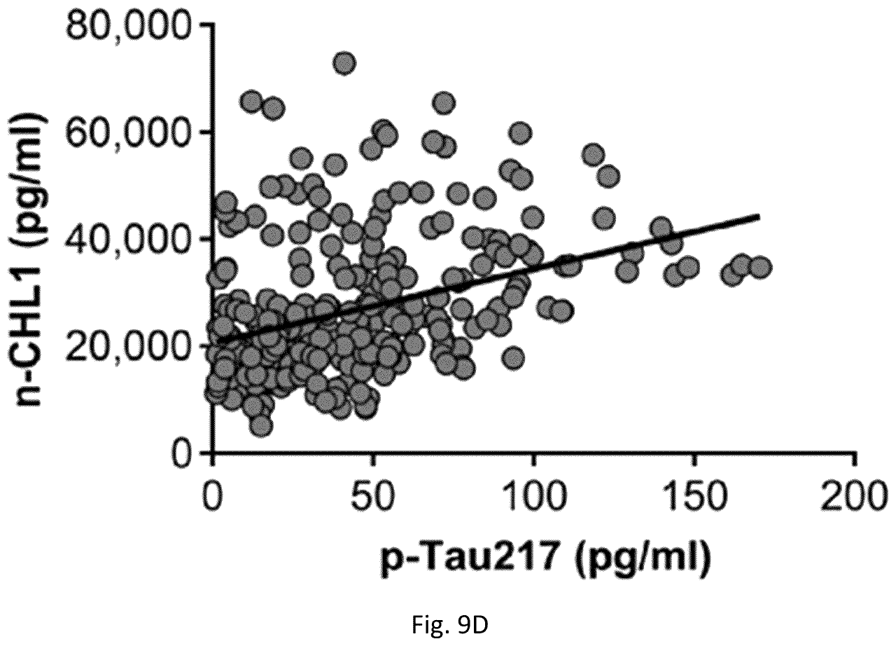

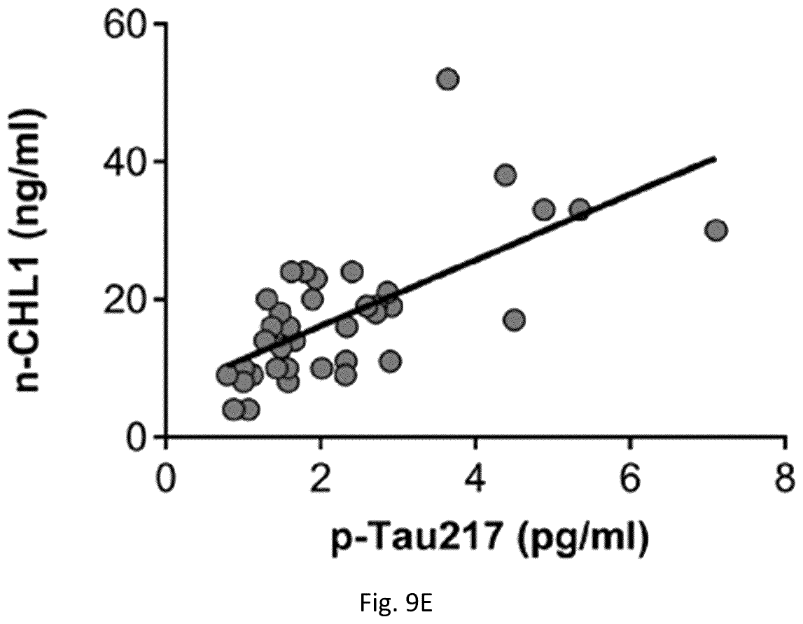

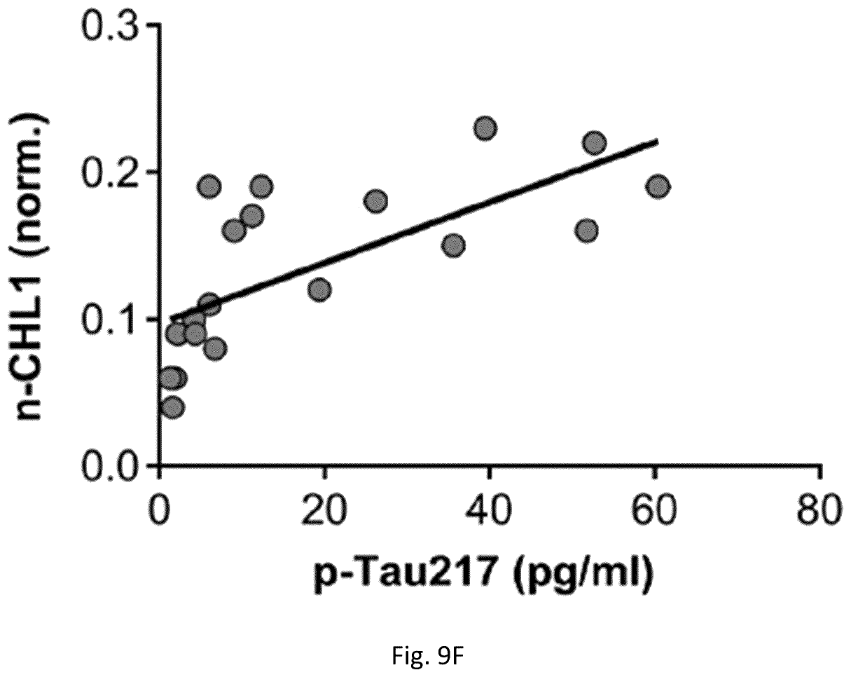

It was next set out to determine the relationship of three proteomic candidates, n-APLP1, n-CHL1 and md-tau in the CSF of 316 human AD patients with mild-to-moderate dementia, 40 cognitively healthy controls, and 21 subjects who were clinically determined to have mild cognitive impairment (MCI). Three outliers of n-APLP1 and n-CHL1 were identified by the SPSS statistics software, using the program's boxplot analysis. In FIG. 8, three patients who did not meet current criteria for “prodromal AD” (subjects clinically diagnosed as MCIs, who are biomarker positive), and “Controls” (cognitively healthy individuals, who are biomarker negative) were excluded from the analysis. Three patients had missing md-tau measurements. In FIG. 9, in the healthy control group, three outliers were identified for p-tau217 and excluded from the analysis.

Vps35^{fl/fl}; Camk2α-Cre knockout mice

All mice were housed in a barrier facility and used in accordance with National Institute of Health and Columbia University Institutional Animal Care and Use Committee (IACUC) regulations. Vps35 floxed mice were generated at Center for Mouse Genome Modification at UConn health. Homologous recombination was performed in mouse embryonic stem cells (mES) targeting the Vps35 gene at exons 2 to 6. The recombined gene had LoxP sites before exons 3 and after exon 5. G418 and Gancyclovir selection and nested long-range PCR were used to identify targeted clones. Targeted ES cells were aggregated into morula to generate chimeric mice. Next the chimeric mice were bred with ROSA26-Flpe to remove the Frt-flanked PGKneo cassette. Neuronal-selective Vps35 knockout mice were generated by crossing mice expressing loxP-flanked Vps35 (exons 3-5) (Vps35^{fl/fl}) with mice expressing Cre recombinase under the 356 Camk2α promoter. Camk2a-CRE mice were obtained from Jackson labs (Stock No: 005359). In all experiments, littermate Vps35^{fl/fl}; Camk2α-Cre (Vps35 cKO) were compared to Vps35^{fl/fl} (Control). Females and males of 3 and 6 months of age were used for all the experiments, with the exception of the initial proteomic screen performed with 6 month-old males.

### Murine Cerebrospinal Fluid (CSF) Collection and Proteomic Screen

Due to the well-known circadian rhythm fluctuations in CSF proteins, all CSF collections were performed within a specific 4-hour time window (generally in the afternoon) that did not vary by cohort or experiment method. For proteomic studies, murine CSF was collected in a post-mortem procedure following CO₂ overdose in accordance with Columbia University IACUC guidelines. Briefly, euthanized mice were placed in a prone position and the skin covering the back of the neck was shaved. A cotton swab containing 70% ethanol was used to remove any hair from the exposed skin. Then, a 27-gauge sterile needle (SV*27EL, Terumo Medical Products) attached to a 1-ml syringe (329650, BD Biosciences) was inserted into the cisterna magna allowing flow of CSF into the butterfly needle. After 10-15 secs, the needle was removed and the CSF aspirated into microcentrifuge tubes (1605-0000, USA Scientific), followed by a brief centrifugation at 600×g for 6 mins at 4° C. Supernatant was transferred to a new tube, immediately placed on dry ice, and further stored at −80° C. Roughly of fluid was collected per mouse. CSF visibly contaminated with blood (pelleted residual erythrocytes) was discarded. All remaining samples underwent more stringent assessment for blood contamination via hemoglobin ELISA (ab157715, Abcam) using 0.5 uL CSF with a 1:200 dilution. Samples below 0.01% blood contamination were retained for biochemical analyses and pooled into biological replicates of 30 uL each for proteomic analysis. Four biological replicates of control samples and five Vps35 cKO samples (each a composite of CSF from 4-7 mice) were generated. The protein concentration of each pooled sample was determined via Qubit Protein Quantification Assay (Q33211, Life Technology), with sample concentrations ranging from 0.10-0.16 mg/ml. Each sample of 4 g of protein was then loaded onto an S-trap column (PROFITI, 382 NY), digested with trypsin, eluted with trifluoroacetic acid, and dried. Two technical replicates (each containing 1.5 ug of tryptic peptides) were run per sample.

Antemortem CSF samples used for the validation of the proteomic hits were collected from anesthetized animals in accordance with Columbia University Institutional Animal Care and Use Committee (IACUC) guidelines. Mice were anesthetized via intraperitoneal injection with a mixture containing ketamine (100 mg/kg body weight) and xylazine (10 mg/kg body weight) before CSF collection as described above.

### LC-MS/MS Analysis

The concentrated peptide mix was reconstituted in a solution of 2% acetonitrile (ACN), 2% formic acid (FA) for MS analysis. Peptides were eluted from the ES 802 column (75 um×25 cm, Thermo Fisher) at 300 nl per minute using a Dionex Ultimate 3000 Nano LC system with a 90-minute gradient from 2% buffer B to 35% buffer B (100% acetonitrile, 0.1% formic acid). The gradient was switched from 35% to 85% buffer B over 1 min and held constant for 2 min. Finally, the gradient was changed from 85% buffer B to 98% buffer A (100% water, 0.1% formic acid) over 1 min, and then held constant at 98% buffer A for 5 more minutes. The application of a 2.0 kV distal voltage electrosprayed the eluting peptides directly into the Thermo Fusion Tribrid mass spectrometer equipped with an EASY-Spray source (Thermo Scientific). Mass spectrometer-scanning functions and HPLC gradients were controlled by the Xcalibur data system (Thermo Fisher Scientific). MS data were acquired in the Orbitrap (FT) at 120,000 resolution from m/z 400-1600. CID MS/MS were acquired in the Ion Trap (IT) on 2+ and higher charge state ions for 3 sec duty cycles.

Database Search and Interpretation of MS/MS Data

The Proteome Discoverer application extracts relevant tandem mass spectra from the raw file and determines the precursor charge state and the quality of the fragmentation spectrum. The Proteome Discoverer probability-based scoring system rates the relevance of the best matches found by the SEQUEST algorithm. The human protein database was downloaded as FASTA-formatted sequences from UniProt protein database (released on July 2017). The peptide mass search tolerance was set to 10 ppm. A minimum sequence length of 7 amino acids residues was required. Only fully tryptic peptides were considered. To calculate confidence levels and false positive rates (FUR), Proteome Discoverer generates a decoy database containing reverse sequences of the non-decoy protein database and performs the search against this concatenated database (non-decoy+decoy). 1% FDR was used to generate the quantitative list for statistical analysis. Total amount of peptides was used to normalize among samples. A total of 1505 mouse proteins were detected and included in the final data set. For each protein, quantification was determined by averaging the peak area of the three most abundant and distinct peptides (if available) and normalized to total area. Qlucore Omics Explorer and Minitab17 Software were used by the Proteomics Core to perform correlation and statistical analyses. Spearman correlations identified one outlier per genotype, removed prior to Downstream Analysis.

### Antibodies and Other Reagents for Mouse Studies

The following antibodies were used for immunoblot; VPS35 (ab57632, mouse, 1:1000, Abcam), Actin (NB600-535, mouse, 1:10000, Novus Biologicals), Albumin (NB600-41532, goat, 1:5000, Novus Biologicals), Albumin (ab19194, goat, 1:10000, Abcam), CHL1 (AF2147-SP, goat, 1:500, Novus Biologicals), CHL1 (25250-1-AP, rabbit, 1:1000, ProteinTech), APLP1 (AF3179-SP, rabbit, 1:500, R&D Systems), Tau (ab80579, mouse, 1:500, Abcam), beta-III tubulin (ab107216, chicken, 1:5000, Abcam), PSD95 (MAB1596, mouse, 1:1000, Millipore), Synaptophysin (MAB5258, mouse, 1:30 000, millipore), anti-mouse HRP (170-6516, Bio-Rad), anti-goat HRP (172-1034, rabbit, 1:3000, Bio-Rad), anti-rabbit HRP (1706515, goat, Bio-Rad), anti-chicken 800CW (925-32218, donkey, 1:20000, Licor), anti-mouse 680LT (925-68022, donkey, 1:20000, Licor), anti-rabbit 800CW (925-32211, goat, 1:20000, Licor), Protein G-HRP (18-161, 1:10000, Sigma Aldrich), Protein A-HRP (101023, 1:10000, Thermo Fisher).

The following antibodies were used for immunohistochemistry; NeuN (ABN78, rabbit, 1:200, Millipore) and VPS35 (ab10099, goat, 1:300, Abcam).

The following ELISA kit was used: Hemoglobin Mouse ELISA Kit (ab157715, Abcam). Roche lumi-light substrate (12015200001, Sigma Aldrich) was used for chemiluminescence.

### Immunoblotting

Samples were prepared in LDS sample buffer (NP0007, Thermo Fisher) and reducing agent (161-0792, Bio-Rad), boiled for 5 minutes at 90° C. and run via electrophoresis in 4-12% bis-tris gels (NP0336 or NP0335, Thermo Fisher) using MES buffer (NP0002-02, Thermo Fisher). For CSF and media experiments, equal volumes (5-15 uL) were loaded in each lane; for lysate, equal masses were loaded in each lane. Proteins were then transferred to nitrocellulose membranes (IB3010-31, Invitrogen) via iBlot (P3; 7:40) and blocked for 1 hour in 3-5% bovine serum albumin (A30075-100 gm, Research Product International) or Odyssey blocking buffer (927-40000, Licor) diluted 1:1 in PBS-T. Membranes were then probed with primary antibodies 448 overnight at 4° C., washed in PBS-T, and probed with secondary antibodies (HRP-conjugated or IR-conjugated) for one hour at room temperature. Membranes were again washed in PBS-T and imaged in Roche substrate (12015196001, Sigma Aldrich) for ECL or alone with the Licor odyssey system. Densitometric values for all proteins were calculated using Image Studio Lite software; reported values are normalized to albumin and β-actin.

### Immunohistochemistry and Histological Analysis

For immunohistochemistry studies, mice (females and males) were anesthetized, rinsed by cardiac perfusion with 0.9% saline buffer, and further fixed with 4% PFA. Whole brains were harvested and post fixed in 4% PFA overnight, followed by cryoprotection treatment in 30% sucrose (wt/vol) in PBS for 16 hours. Tissue blocks were then embedded in Optimal Cutting Temperature (OCT) compound and stored at −80° C. Briefly, 30 μm free-floating horizontal sections were washed three times with PBS and incubated at 4° C. overnight on a rotator in 1 ml of primary antibody (VPS35 or NeuN) diluted in PBS containing 0.3% Triton X-100 (vol/vol) and 5% normal serum (vol/vol). After three washes with PBS-T (0.1% Triton X-100), the sections were incubated for 30 minutes with secondary antibodies (Molecular probes). Following three washes with PBS-T, and one wash with PBS, sections were mounted on slides. Fluorescent images were collected using a Zeiss LSM 700 META confocal microscope equipped with a 63x Plan-Apochromat objective and HeNE1, HeNe2 and argon lasers. A semi-quantitative count of NeuN-positive neurons in the CA1 hippocampal region was performed in 6 Vps35 cKO mice and 6 control littermates. 4-5 Spaced NeuN-stained horizontal sections, starting from Bregma −2.28 to Bregma −3.60, were analyzed per animal using an automated cell counting plugin (http://imagej.net/Particle_Analysis) on ImageJ software (version 1.48, US National Institutes of Health). Briefly, evaluation of cell density was made on z projections of 10 consecutive confocal scans (total thickness 10 μm). A fixed threshold value was applied to all sections in order to identify the NeuN-positive neurons. Number of cells were counted in the CA1 region manually delineated on each individual section, and area of analysis was measured. The density of immunolabeled neurons was expressed as the mean number of positive cells per square millimeter (number/mm 2). All analysis was performed by a scientist blinded to the genotype status of the animal.

Histological Nissl stainings were performed according to the manufacturer's instructions (IW-3007, NovaUltra Nissl Staining kit, IHCWORLD). Quantification of the neuronal density was performed on 4-5 sections per animal (n=6 mice per group) starting from Bregma −2.28 mm to Bregma −3.60 mm. As for the NeuN quantification, the hippocampal cell layer CA1 were delineated on each cresyl violet-stained sections. Slides were scanned using a MPSR Leica SCN400 slide scanner (40× objective), and further analyzed using the built-in “Multi-point Tool” on ImageJ to manually count the stained cells. Briefly, cell density values were obtained by dividing the number of immunoreactive neurons by the total area of the corresponding hippocampal CA1 region (number/mm 2).

### Md-Tau Quantification in Murine CSF

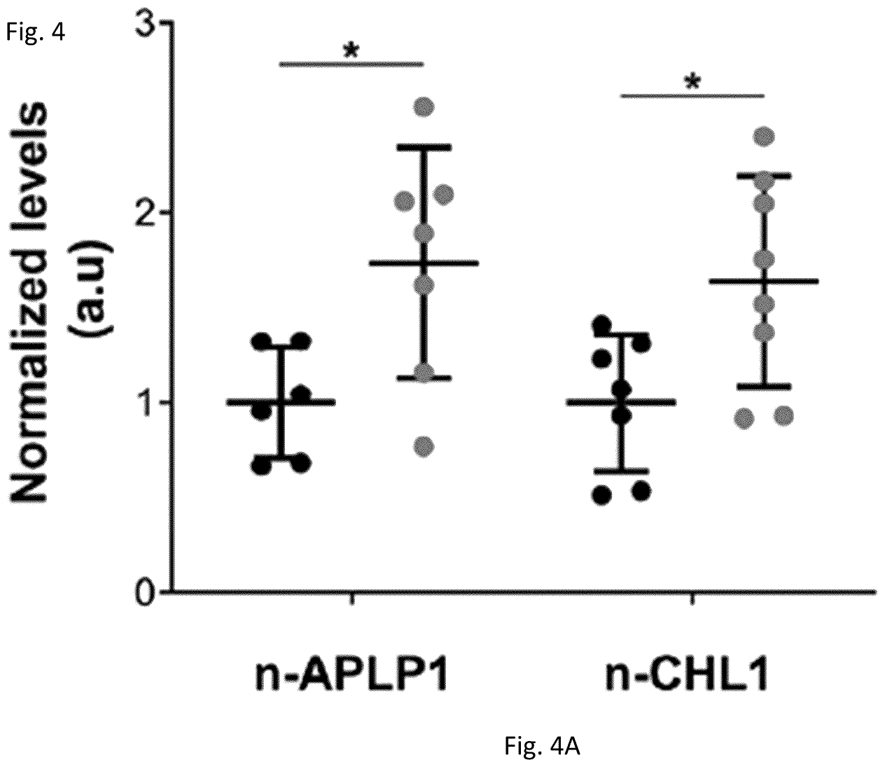

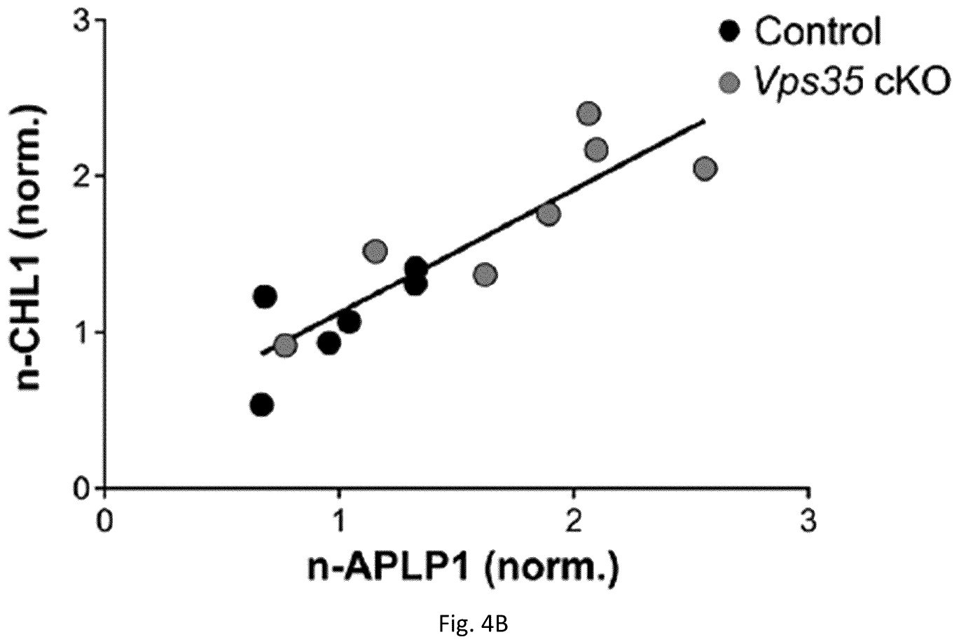

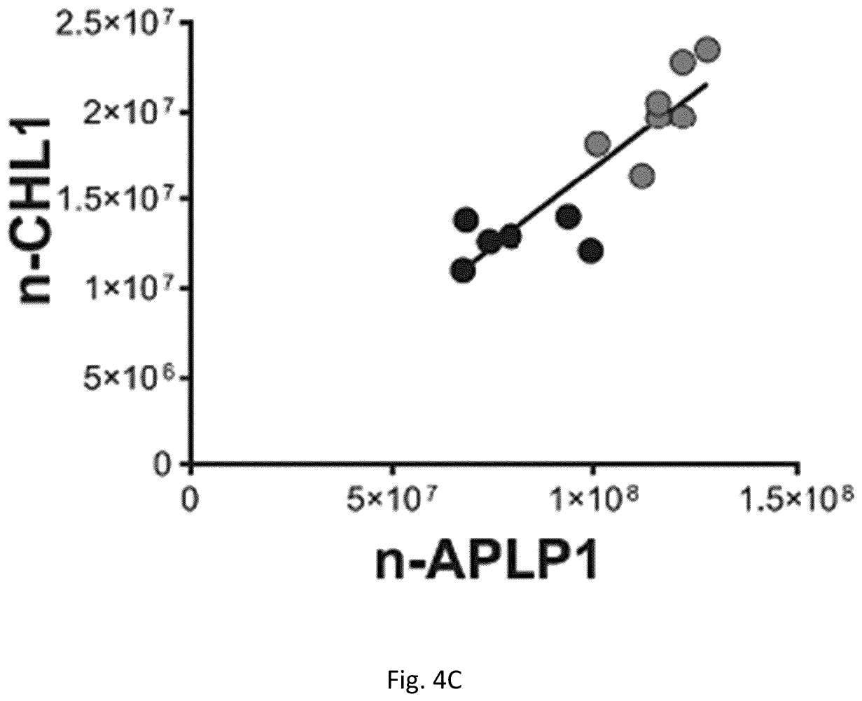

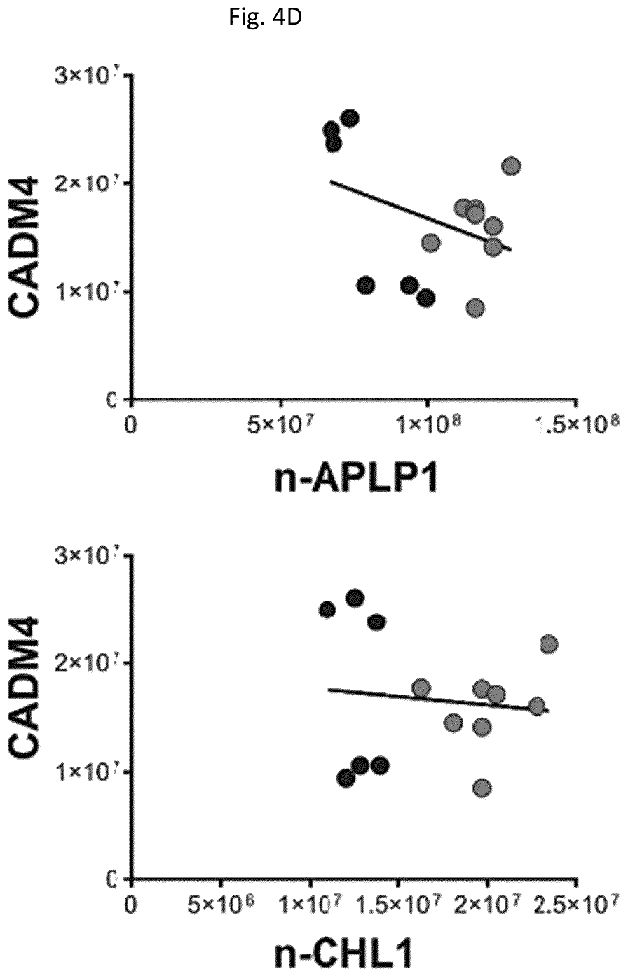

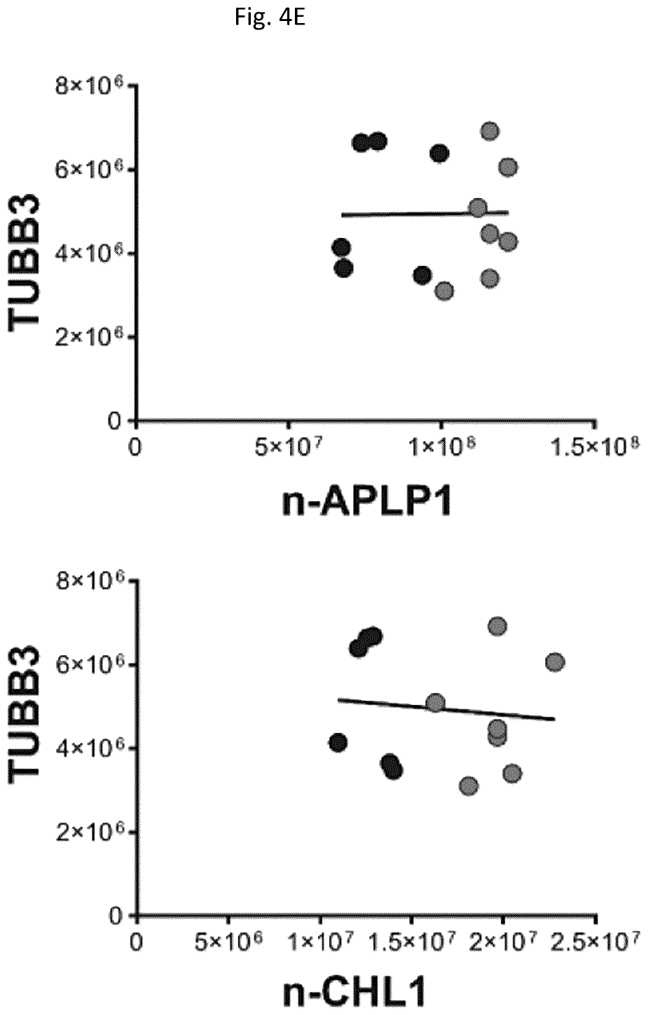

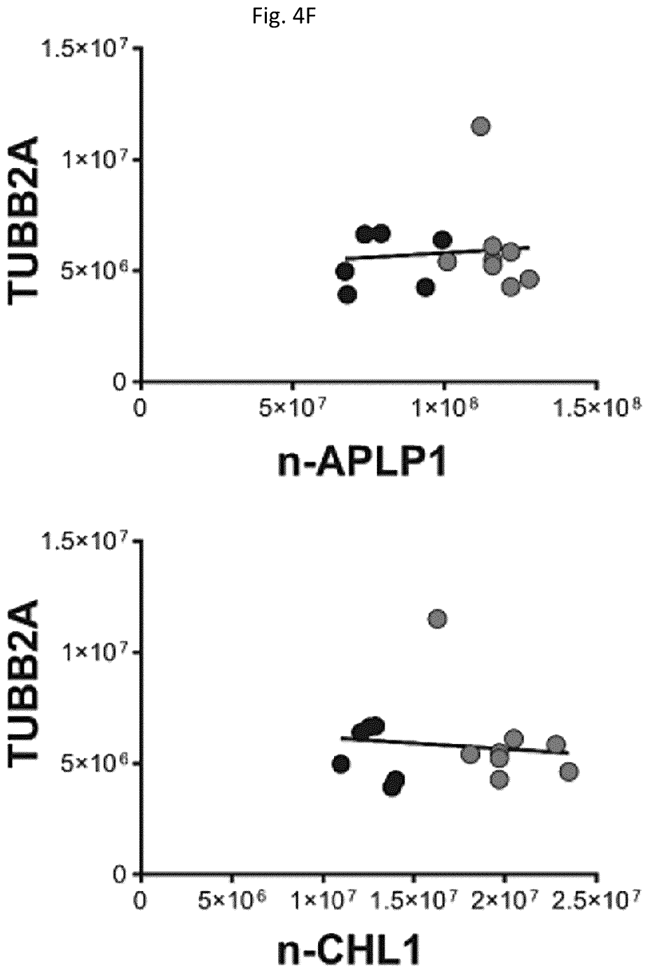

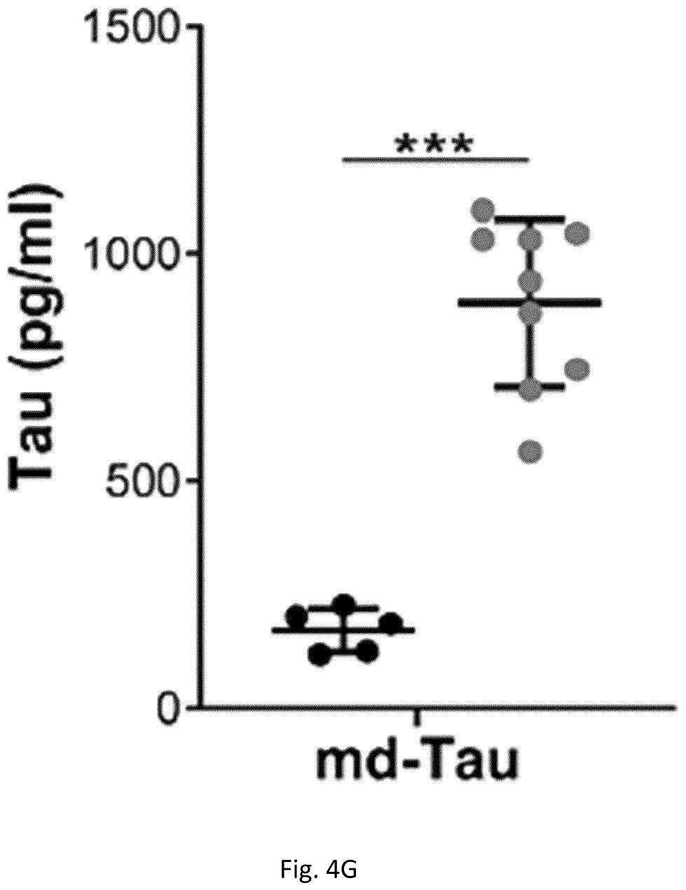

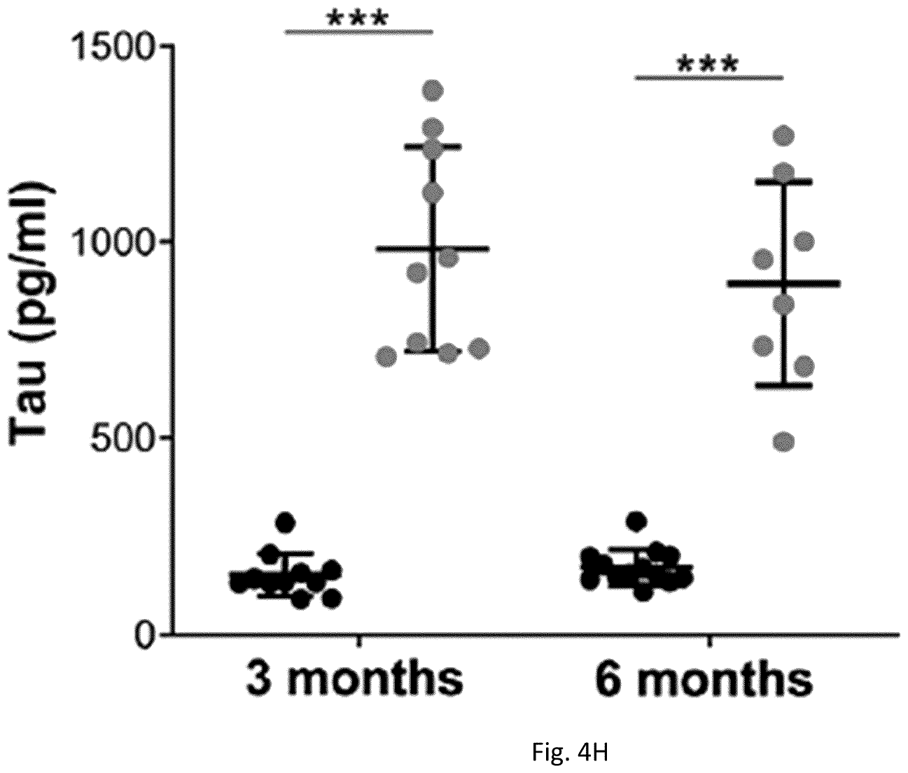

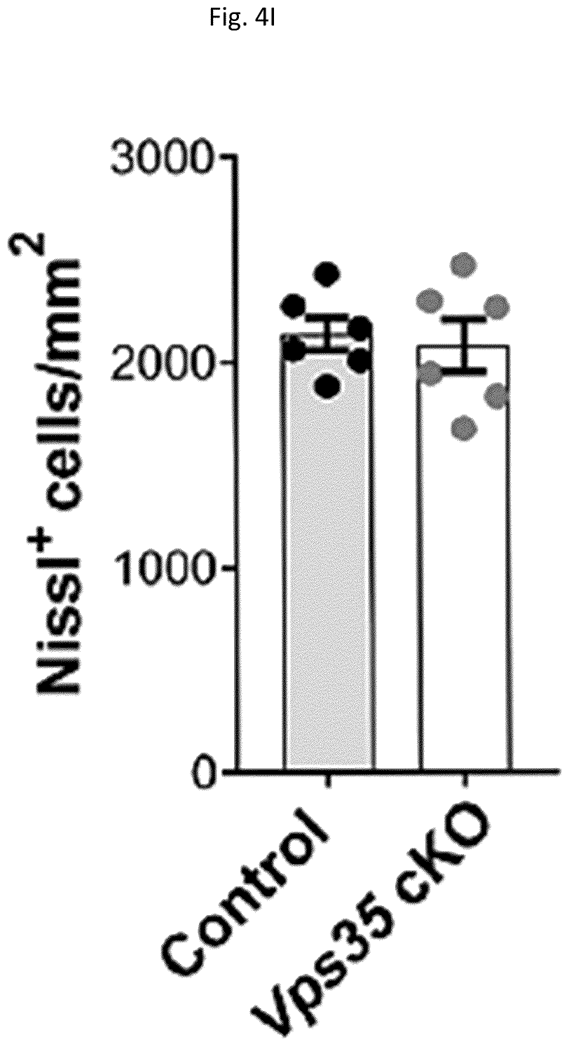

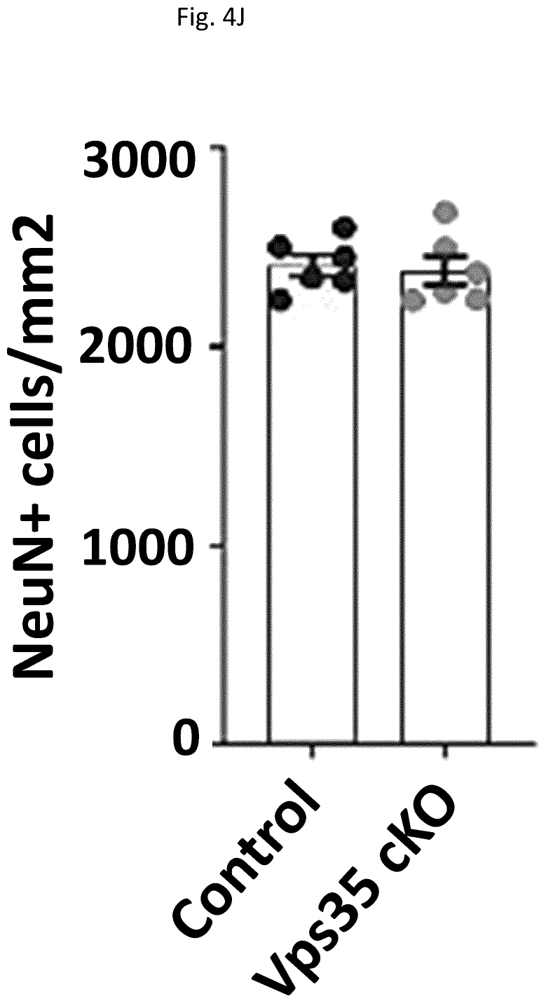

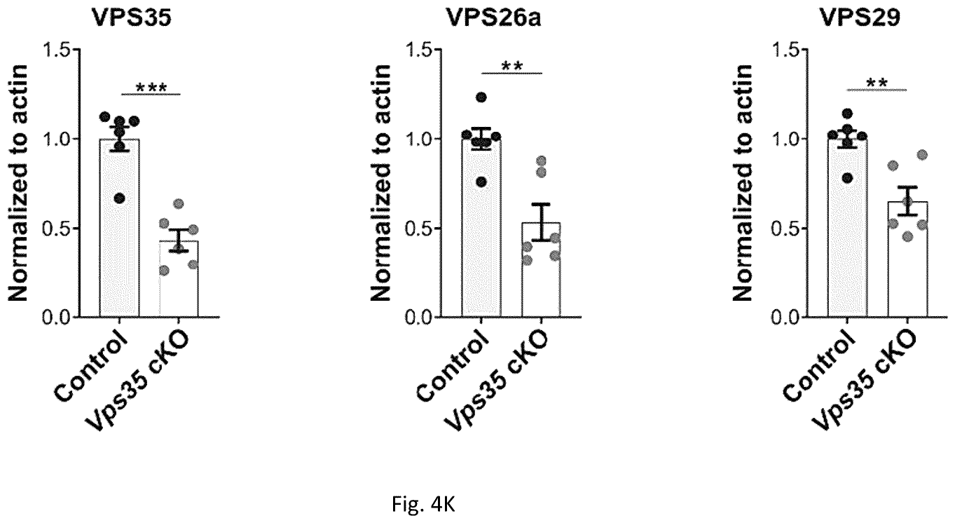

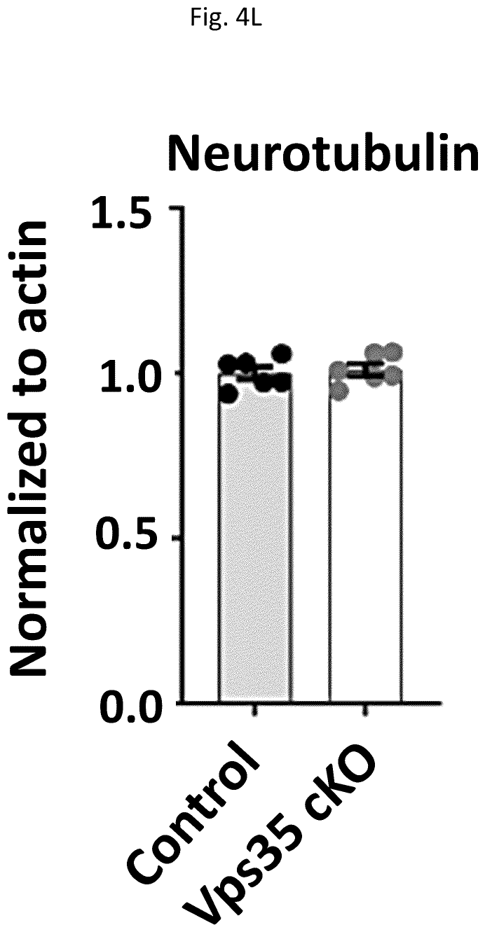

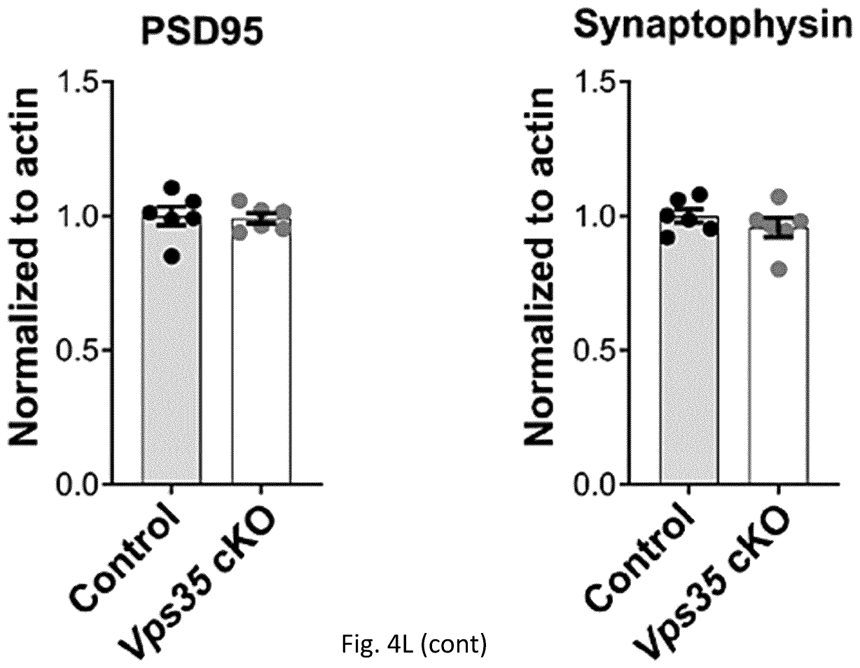

In FIG. 4G, postmortem CSF tau was quantified by Simoa™ technology using the mouse total-tau assay (102209, Quanterix), which is directed against the mid-protein epitope of the tau holoprotein. Individual CSF samples (5 uL) were diluted 1:80 in sample buffer and then split into technical duplicates. Standards and samples were run according to the manufacturer's instructions and read on the HD-1 analyzer (Quanterix). Replication of CSF tau elevation in 3 and 6 month-old mice shown in FIG. 4H was performed using antemortem CSF samples collected from two different cohorts (22 females and 20 males) of anesthetized animals in accordance with Columbia University IACUC guidelines. For the latter, individual CSF samples (2 uL) were diluted 1:100 in sample buffer and then split into technical duplicates and read on the SR-X analyzer (Quanterix). CSF samples from Tau KO mice were used as negative controls.

n-APLP1 and n-CHL1 Quantification in Human CSF Assay Development

The following 8 APLP1 antibodies were screened for compatibility; 1=#354008 (ThermoFisher), 2=AF3129 (NovusBio), 3=AF3179 (NovusBio), 4=MAB3908 (NovusBio), 5=12305-2-AP (ProteinTech), 6=MAB3129 (ProteinTech), 7=ab210557 (AbCam), and (AbCam) using extracellular domain APLP1 recombinant protein #AG2951 (ProteinTech) using Simoa™ homebrew kit (cat #101354, Quanterix) and standard assay definitions.

The following 6 CHL1 antibodies were screened for compatibility; 8=MABN229 (Millipore), 9=AF2147 (NovusBio), 11=MAB2126 (NovusBio), 12=25250-1-AP (ProteinTech), 13=MAB2147 (NovusBio), and 14=LS-C485419 (LsBio) using membrane proximal extracellular domain CHL1 recombinant protein #AG19263 (ProteinTech) using Simoa™ homebrew kit (cat #101354, Quanterix) and standard assay definitions.

The top 6 assays for each analyte (most sensitive with recombinant protein) were used to measure titrations of low tau (healthy control) CSF pool to confirm recognition of endogenous signal, and dilution linearity. The top 2 assays for each analyte from the CSF screen were then optimized to improve sensitivity. The final assay chosen for each analyte (APLP1:2×10 and CHL1:12×9) were used for subsequent clinical cohort 25 measurements. CSF was diluted to 1:80 for n-APLP1 measurement, and 1:50 for n-CHL1 measurement.

### Human CSF Studies

CSF from healthy controls and MCI subjects were requested from the Columbia University Alzheimer's Disease Research Center (ADRC) CSF bank. This CSF bank includes CSF obtained by lumbar puncture and banked for research according to protocols approved by the Columbia University IRB. CSF was stored frozen in 400 uL aliquots in polypropylene tubes, at −80° C. Frozen CSF aliquots were later thawed and analyzed for Aβ42 and total tau by the ADRC Clinical Core using a multiplex fluorimetric microsphere xMAP bead-based sandwich immunoassay consisting of monoclonal antibodies covalently coupled to spectrally specific fluorescent beads. This assay (Inno-BIA AlzBio3, Fujirebio US Inc.) was performed on a Luminex 100 machine. Thresholds implemented to confirm amyloid positivity in AD patients were CSF total tau/Aβ42≥0.15. CSF from mild-to-moderate dementia subjects, was obtained from Janssen clinical trial ELN115727-301/302. These lumbar puncture samples, obtained with Informed consent for use in AD biomarker research, were stored in 500 ul aliquots in polypropylene tubes at −80° C. until use. All measurements shown (Innotest Aβ42: plaque positivity, Aβ42=600 pg/ml) and in-house Simoa™ n-APLP1, n-CHL1, md-Tau, and p-tau217 were performed at Janssen R&D are described in Triana-Baltzer et al. 2020.

### Statistical Methods

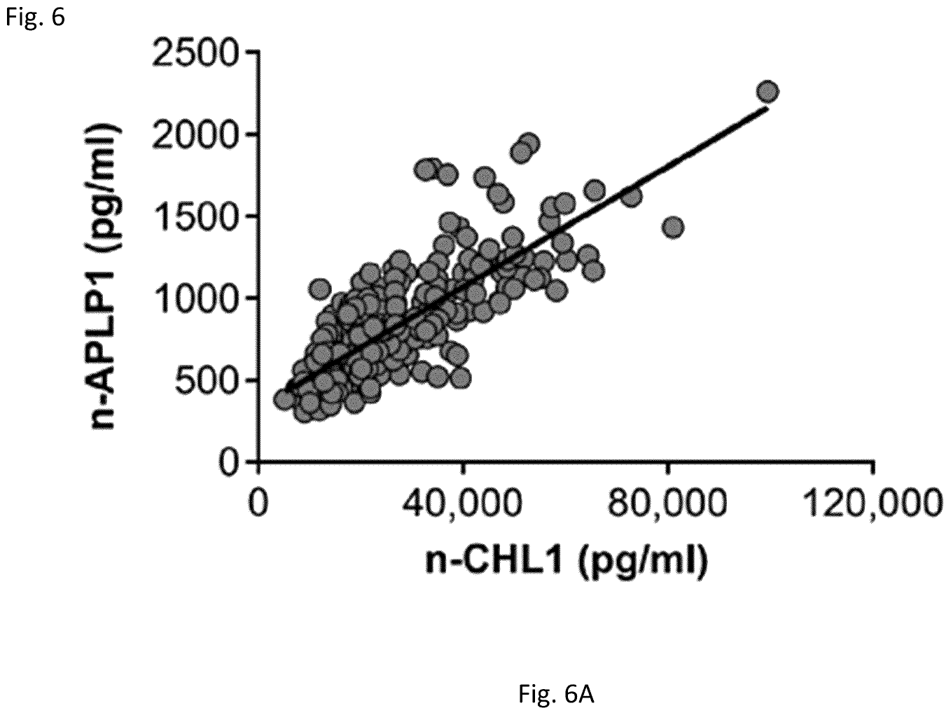

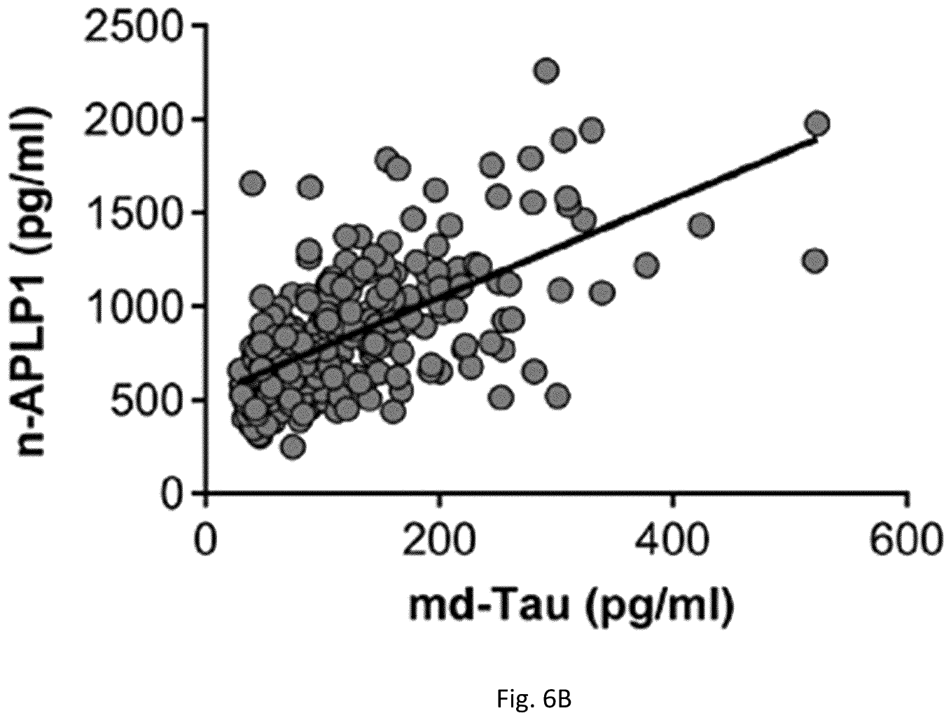

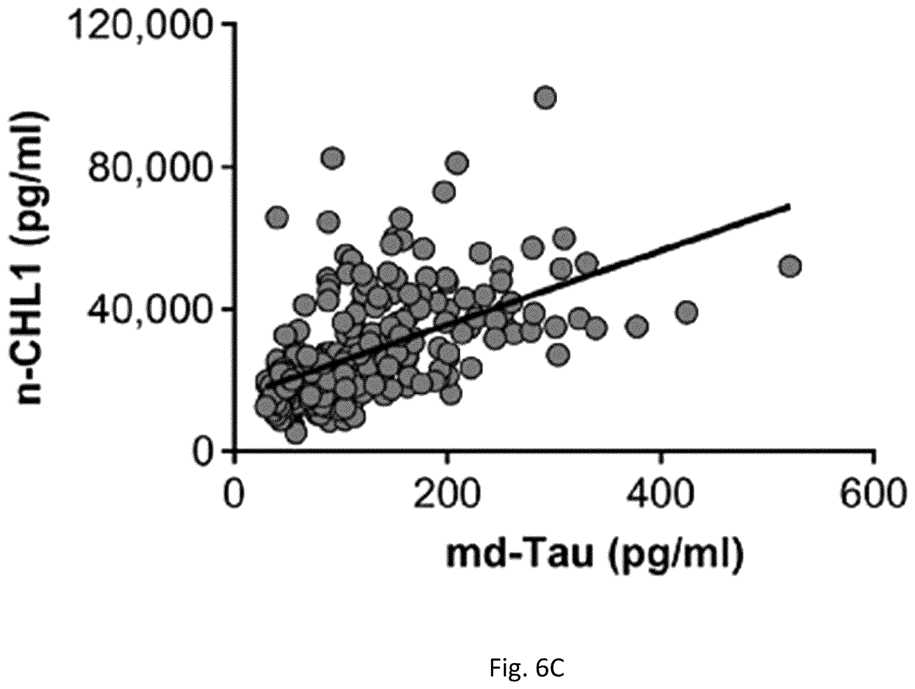

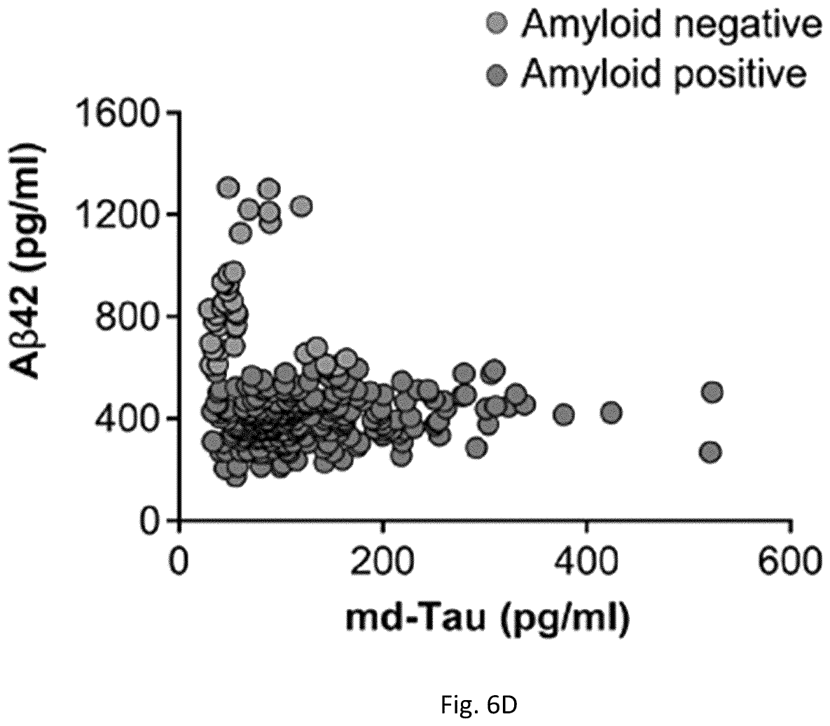

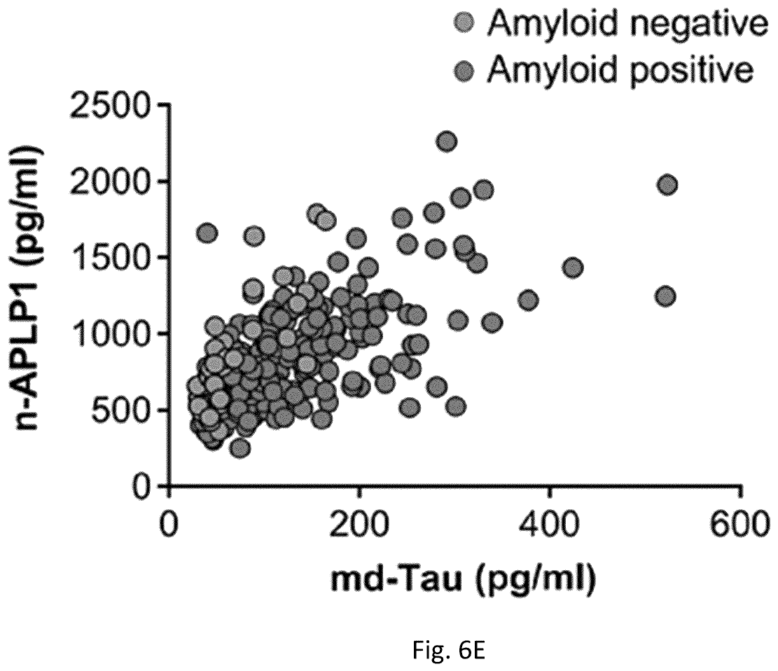

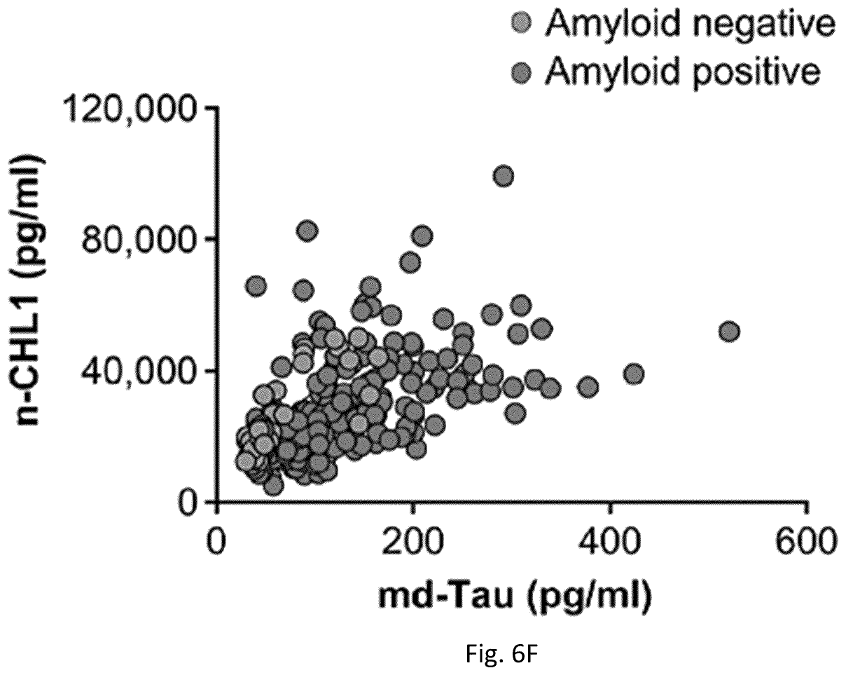

Formal boxplot analysis was applied to each dataset to exclude outliers. Student's t-test was used for all biochemical experiments with a two-tailed distribution with equal variance (P<0.05) or as stated in the figure legend. GraphPad Prism version 7.00 for Windows (GraphPad Software) was used for all graph representation, and the statistical analysis, except those shown in FIGS. 6-9. ANOVA was used for parametric analysis of CSF proteomics, and Benjamini and Hochberg False discovery rate (FDR) —corrected p- and q-values are reported. Nonparametric analysis identified proteins which were present in at least 4 technical replicates from one genotype and absent from all replicates of the other genotype. Significance is indicated as *P<0.05, **P<0.01 and ***P<0.005. The SPSS Statistics software was used for human correlation studies. All data are shown as means±SEM or SD. “n” for each experimental design is indicated in figure legends.

### Example 2—Screening the CSF Proteome in Neuronal-Selective Vps35 Knockout Mice

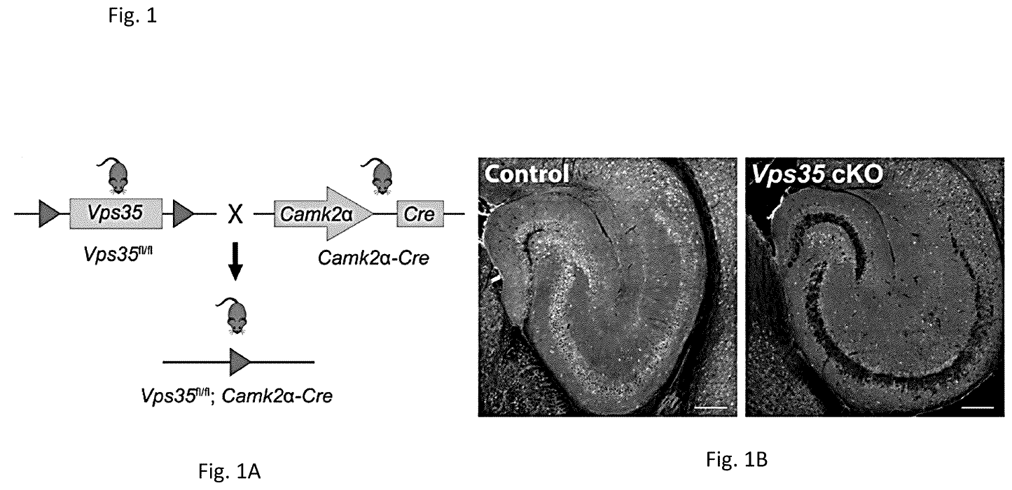

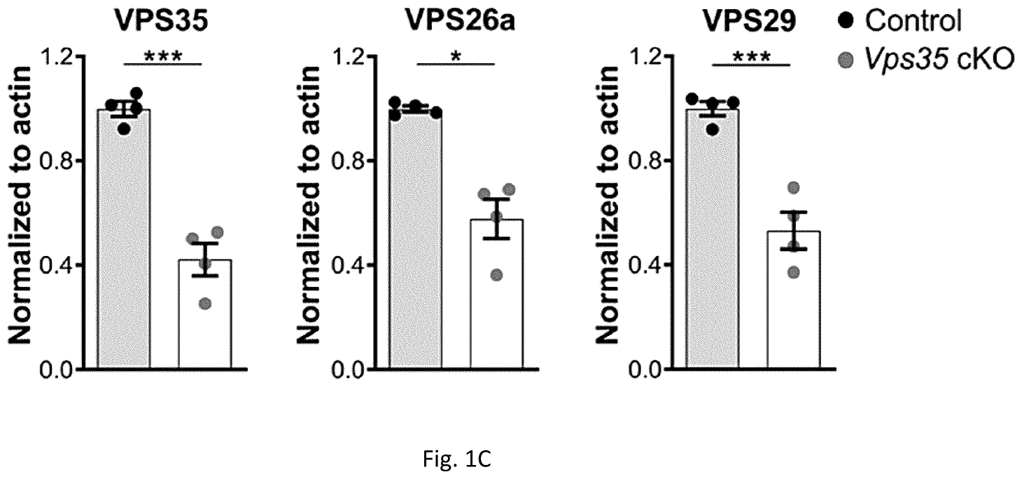

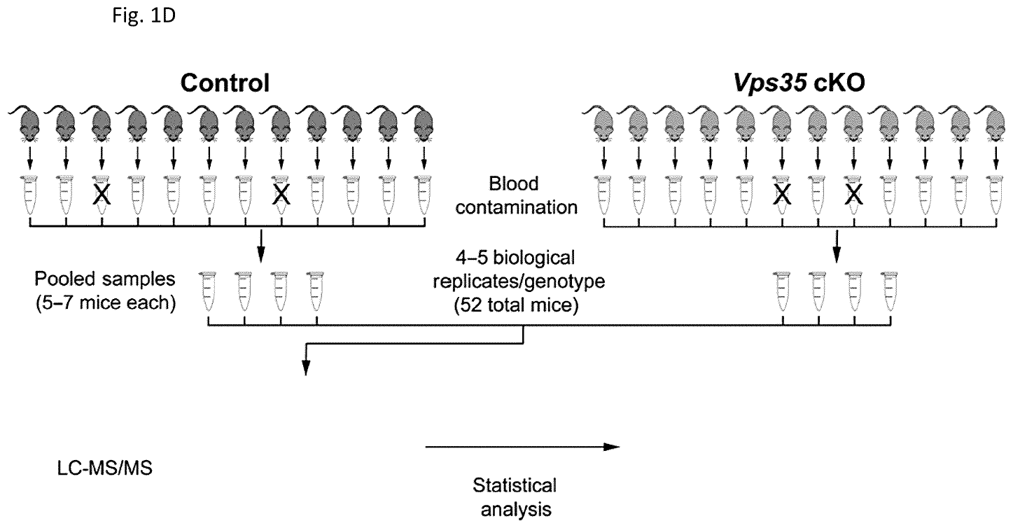

Mice expressing loxP-flanked Vps35 (Vps35^{fl/fl}) on a C57BL/6 background were crossed with mice expressing Cre recombinase under the Camk2α promoter to knockout Vps35 in forebrain neurons (FIG. 1A). In all experiments, Vps35^{fl/fl}; Camk2α-Cre (Vps35 cKO) were compared to Vps35^{fl/fl} (Control). First this mouse model was characterized using confocal microscopy and Western blot analysis Immunofluorescence studies revealed a clear depletion of Vps35 in CA1 pyramidal neurons of Vps35 cKO mice compared to their control littermates (FIG. 1B).

The hippocampus from 6 month-old mice was harvested and it was found by Western blot analysis a concomitant reduction of VPS35 (P=0.0002), and other retromer core proteins, VPS26a (P=0.0286) and VPS29 (P=0.0008) (FIG. 1C).

Proteomic analysis (illustrated in FIG. 1D) identified 1505 proteins in the CSF by one unique peptide (1048 proteins by two unique peptides).

### Example 3—Identifying Alterations in CSF Proteins in Neuronal-Selective Vps35 Knockout Mice

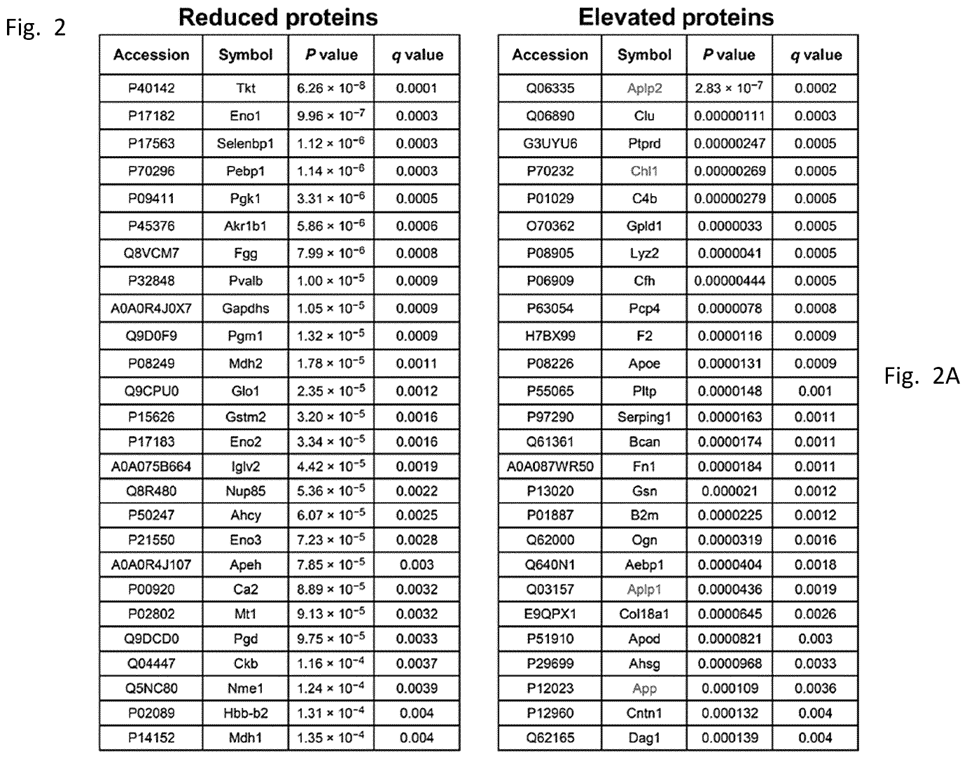

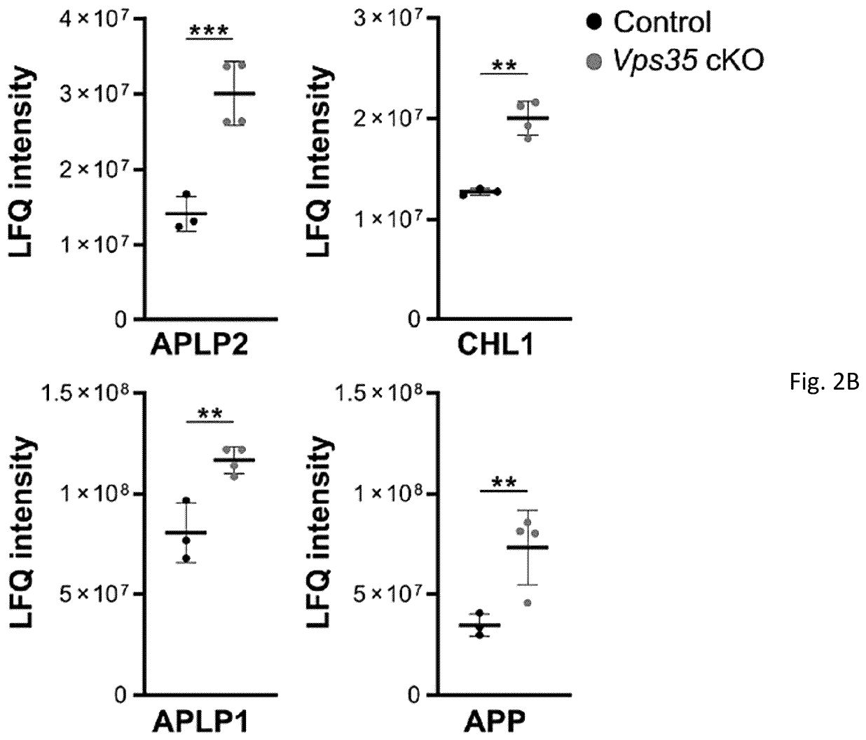

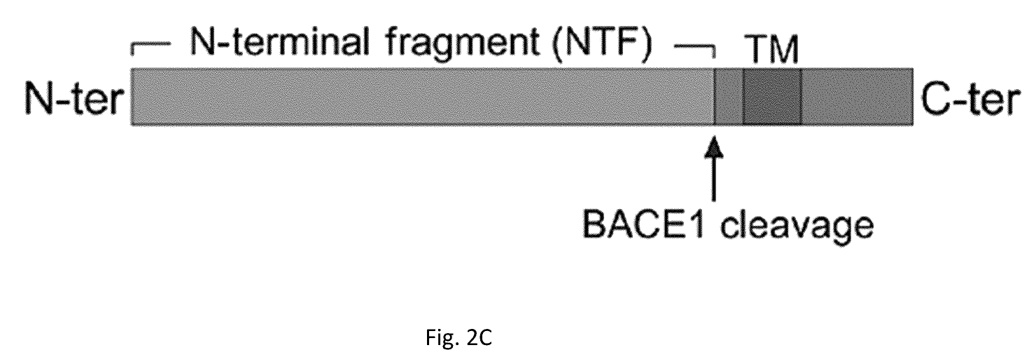

Different analytic approaches were applied to the CSF proteomics data to identify CSF proteins that abnormally accumulate as a result of depleting neuronal Vps35. A parametric approach was first applied, using an ANOVA to compare the difference of mean expression in detected proteins between the Vps35 cKOs and controls. Fifty-two proteins were found significantly altered in the CSF of the Vps35 cKO mice (FIG. 2A). Among the class of elevated proteins, four are type-I transmembrane proteins that are established BACE1 substrates: APLP1, APLP2, APP, and CHL1 (FIG. 2B). BACE1 functions primarily at endosomal membranes where it cleaves its substrates (Vassar et al. 1999) (FIG. 2C), liberating their n-terminal fragments into the endosomal lumen from where these fragments are secreted to the extracellular space. In fact, this group 88 of substrates has been reported as among the top proteins that were found reduced in a CSF proteomic screen of Bacel KO mice (Dislich et al. 2015). Vps35 deficiency has been shown to increase BACE1 activity by increasing the resident time of the enzyme and its substrates in endosomal membranes (Wang et al. 2012; Vardarajan et al. 2012). By investigating the peptides identified by mass spectrometry (MS/MS) it was found that they mapped onto the protein's N-terminal domains, consistent with accelerated retromer-dependent endosomal secretion.

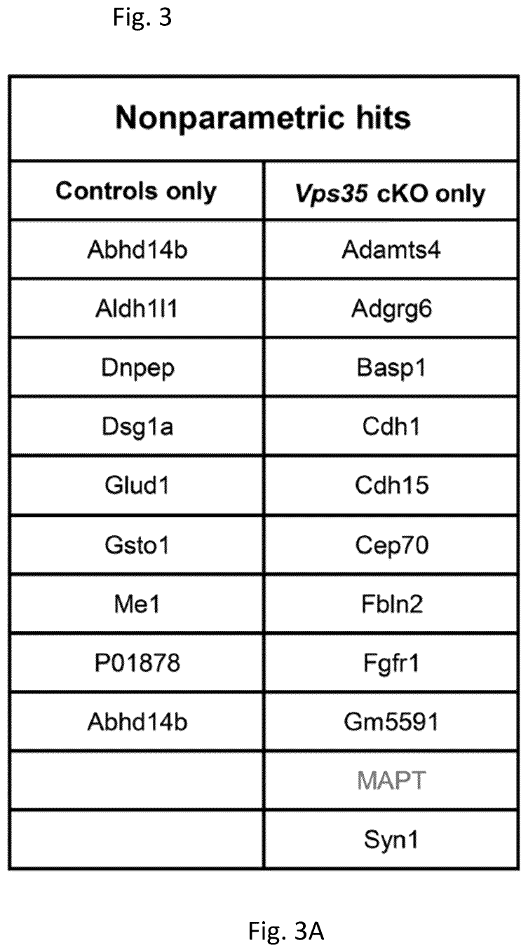

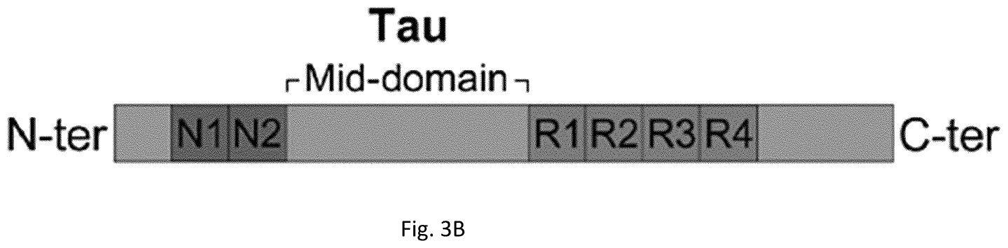

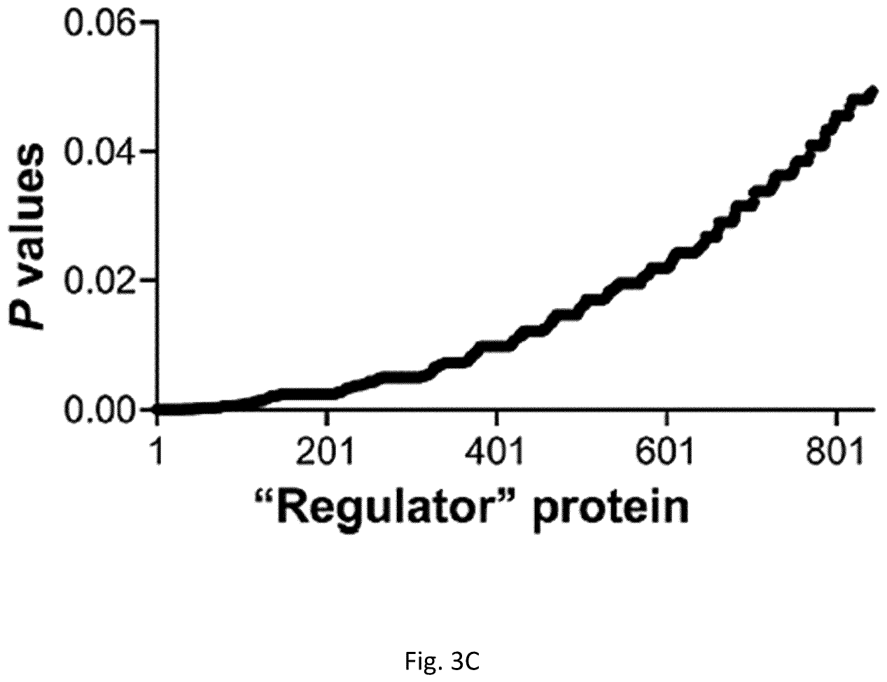

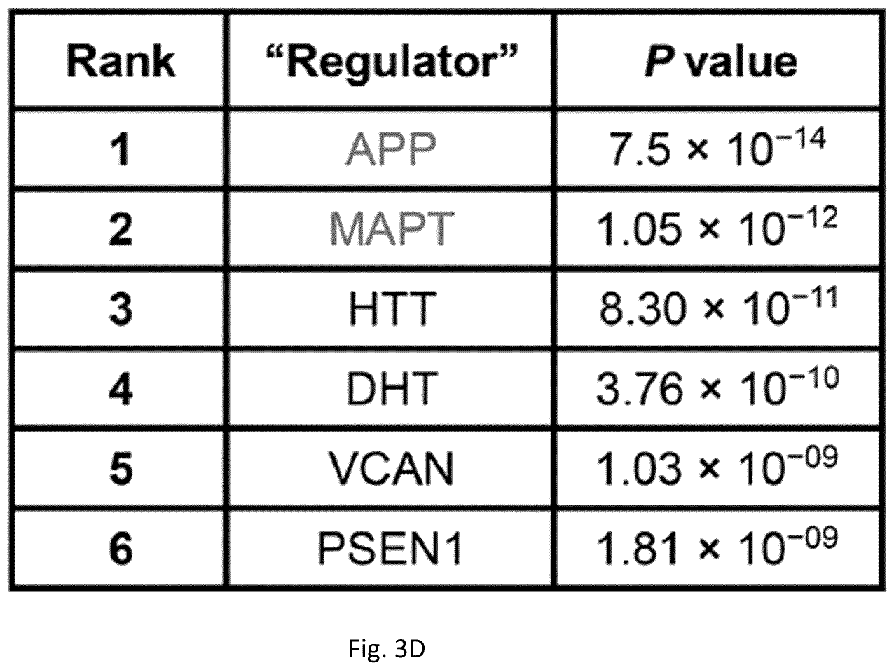

A second, nonparametric, analysis was performed to identify proteins detected in CSF from one genotype but not the other. Eight proteins were detected in the CSF of controls and not the Vps35 cKOs, whereas eleven proteins were detected in the CSF of the Vps35 cKOs and not the controls (FIG. 3A). The microtubule-associated protein tau (MAPT) was one of the proteins detected only in the CSF of Vps35 cKO mice. By investigating the sequence of the tau peptide identified by MS/MS—SGYSSPGSPGTPGSR (SEQ ID NO: 11), it was found that it mapped onto the protein's mid protein region, a domain contained within the N-terminal tau fragment most commonly found to accumulate in the CSF of AD patients (Ssiti et al. 2018; Cicognola et al. 2019; Meredith et al. 2013; Barthelemy et al. 2020a; Barthelemy et al. 2020b) (FIG. 3B).

The final approach relied on ingenuity pathway analysis (IPA) (Kramer et al. 2014) to identify molecules that might be ‘regulating’ the global changes in the CSF of Vps35 cKO mice. This software utilizes established transcriptional interactions to predict upstream molecular convergence of global changes in large datasets (Kramer et al. 2014). Over 800 proteins were identified as potential regulator proteins in this data set (FIG. 3C). The two top regulators with the lowest p-values (>1×10⁻¹¹) were APP and MAPT (FIG. 3D), further supporting the first two analyses performed.

### Example 4—Validating BACE1 Substrates and Tau Accumulation in Mouse CSF

In anticipation of the human CSF studies, it was decided to further confirm and validate the n-APLP1 and n-CHL1 findings. Because these two proteins are highly expressed in the brain. CSF was collected from a separate cohort of Vps35 cKO and control mice and n-CHL1 and n-APLP1 measured via immunoblotting.

Replicating and validating the CSF screen, both n-CHL1 (P=0.02) and n-APLP1 (P=0.02) were significantly elevated in the new cohort of Vps35 cKO CSF as compared to control littermates (FIG. 4A). To investigate the relationships among CSF n-APLP1 and n-CHL1, the mouse CSF data was revisited, and a strong correlation was observed between CSF n-APLP1 and n-CHL1 (n=14, β=0.89, P<0.0001) (FIG. 4B). In individual genotypes and collectively as a group, the relationship was nearly collinear across mice, with the Vps35 cKO mice simply shifting this relationship to the right. These observations suggested that the concentration of these proteins in CSF seem to be regulated by the same endosomal-based source. The same relationship was observed when a correlation analysis was performed using the MS/MS peak intensity measurements (FIG. 4C).

The next aim was to identify neuronal proteins which release into the CSF that are not affected by neuronal Vps35 depletion, as another way of validating the proteomic findings. Two candidates were identified: the synaptic adhesion molecule 4 (CADM4), highly expressed at the cell surface of neurons; and the neuronal tubulin beta3 (TUBB3). Correlation analysis using the MS/MS raw peak intensity measurements (FIGS. 4D and 4E), revealed no relationships between these proteins and n-APLP1, or n-CHL1. CSF concentrations of tubulin 2a (TUBB2a), another cytoskeleton protein generally found in CSF, was not affected by Vps35 depletion (FIG. 4F).

Although a growing body of evidence suggests that tau might also be secreted via the endosomal pathway (Chai et al. 2012; Karch et al. 2012; Mohamed et al. 2014), in contrast to BACE1 substrates, the link between tau and the endosomal pathway in general and to retromer trafficking in particular is still unclear. Quantitation of endogenous tau in mouse CSF is challenging, as CSF concentrations are roughly 50,000-fold lower than that of brain tissue. Therefore, Single-molecule-array (Simoa™) technology, a highly sensitive assay (Rissin et al. 2010) which has been used by prior studies to reliably detect murine CSF tau (Chen et al 2019) was used. The assay employed antibodies directed at the mid-domain epitope of tau (md-Tau), which are commonly used to measure total tau in human patients (Meredith et al. 2013). md-Tau was reliably detected in as little as 2-5 uL of murine antemortem CSF. Furthermore, the Simoa™ measurement of CSF tau in a new cohort of mice revealed a striking increase in the Vps35 cKOs, replicating and validating the initial nonparametric finding (P=0.00002) (FIG. 4G).

Additionally, studies in patients and animal models have established that neuronal cell death is by itself neither necessary nor sufficient to cause elevation in CSF tau. Nevertheless, biochemical and histological analyses was performed in two additional cohorts of Vps35 cKO mice and control littermates to address this issue. The study was started by replicating the findings of elevated CSF tau at 3 and 6 months using antemortem CSF (FIG. 4H). A semi-quantitative analysis by Nissl staining further revealed no signs of neuronal loss in the hippocampus of 3 month-old Vps35 cKO mice, compared to their control littermates (FIG. 4I), which was further supported by NeuN immunofluorescence staining analysis (FIG. 4J). Furthermore, protein expression of pre- and post-synaptic markers were also unaffected in Vps35 151 cKO mice compared to their control littermates (FIGS. 4K and 4L).

Together, these studies suggested that the elevation of CSF tau observed in Vps35 cKO mice is not driven by neuronal cell death.

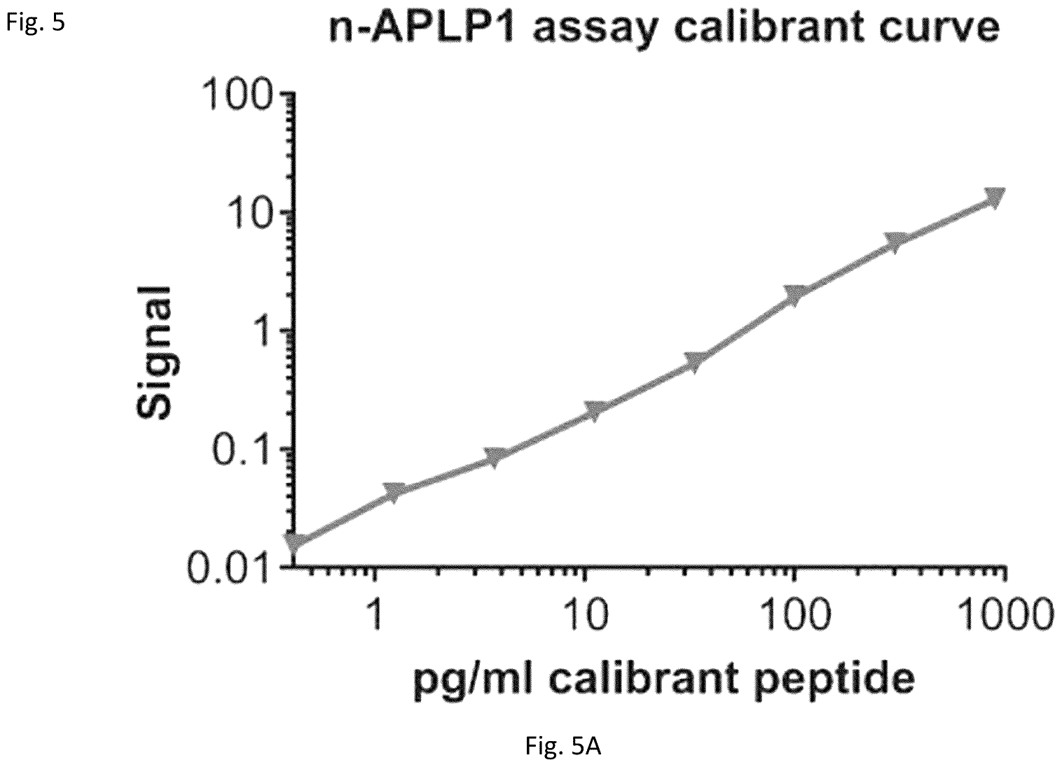

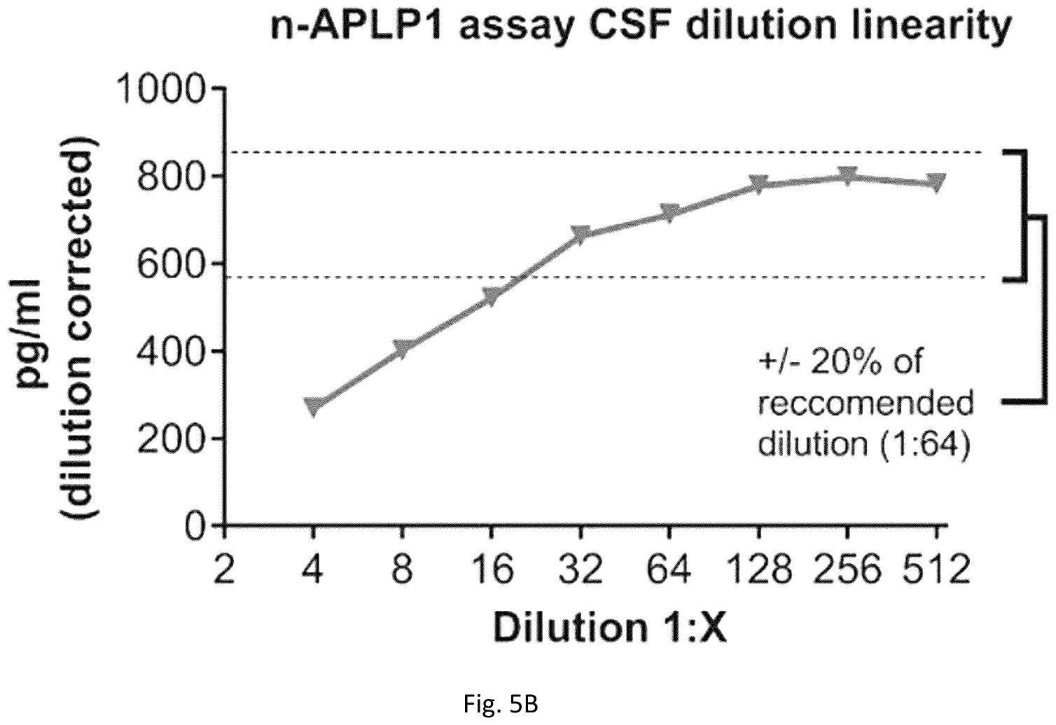

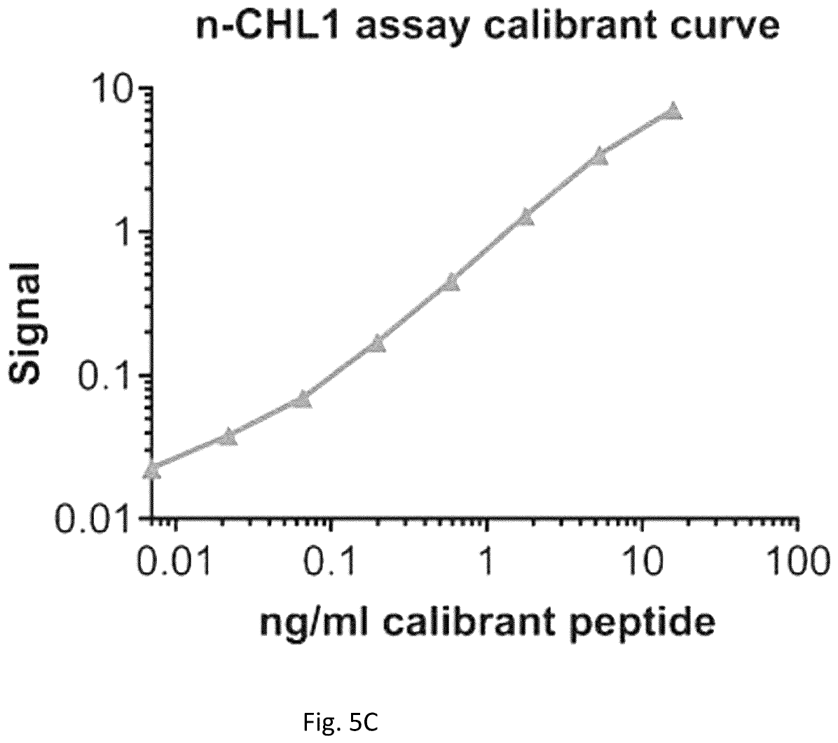

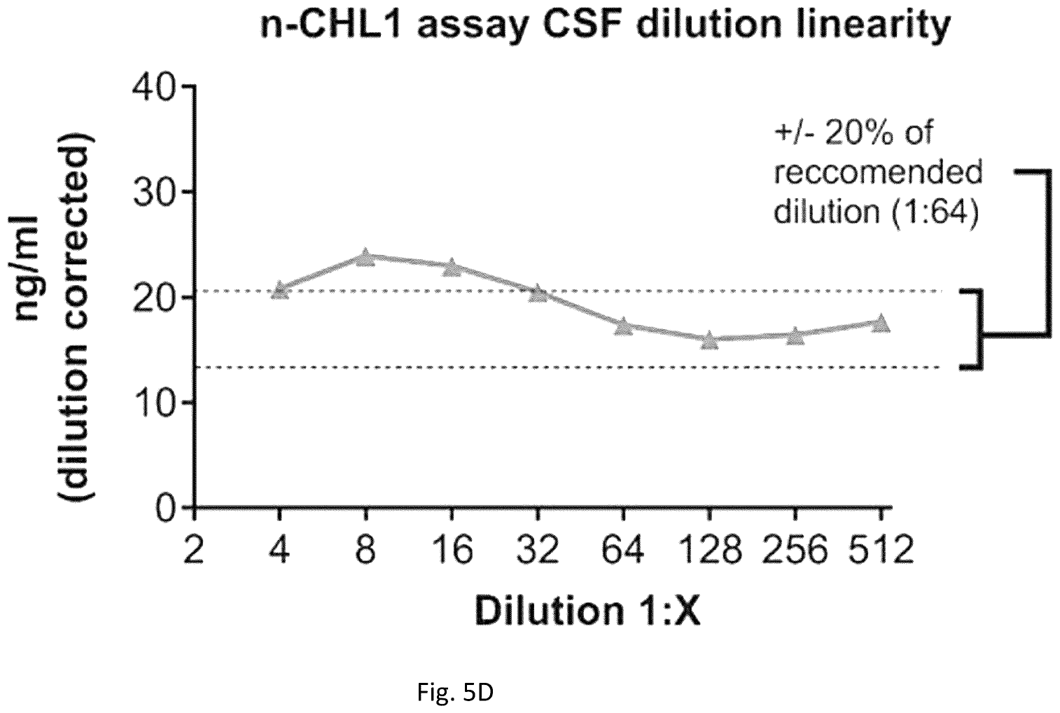

### Example 5—Developing and Validating n-APLP1 and n-CHL1 Simoa™-Based Assays

Whereas validated assays for human md-Tau already exist, in order to measure APLP1 and CHL1 concentrations in humans, Simoa™ assays for n-APLP1 and n-CHL1 were developed and validated. First eight commercially available antibodies directed against the n-terminus of APLP1, and six commercially available antibodies directed against the n-terminus of CHL1 were identified. Using Simoa™ platform, antibody pairs were screened for compatibility via signal with recombinant peptides of either n-APLP1 or n-CHL1. The pairs with highest sensitivity were then evaluated using titrations of healthy control (HC) CSF pool, ultimately yielding several APLP1 and CHL1 assays with good precision, sensitivity and dilution linearity. The best performing n-APLP1 assay exhibited mean intra-test precision of 4.6%, dilution linearity of CSF in range of 1:32-1:512 and reported concentrations of ˜700 pg/ml in HC CSF (FIGS. 5A and 5B). The best performing n-CHL1 assay exhibited mean intra-test precision of 7.6%, dilution linearity of CSF in range of 1:32-1:512 and reported concentrations of ˜15 ng/ml in HC CSF (FIGS. 5C and 5D).

Together, these studies generated Simoa™ assays that are suitable for reliably identifying n-APLP1 and n-CHL1 in human CSF.

### Example 6—n-APLP1 and n-CHL1 are Correlated with Tau in the CSF of Dementia Patients

Next it was sought out to determine the relationship of the three validated hits in the CSF of AD patients with mild-to-moderate dementia. The Simoa™-based assays of n-APLP1 and n-CHL1 were used to measure their concentrations in the CSF of over 316 patients with mild-to-moderate dementia (79% amyloid positive). To compare to traditional AD biomarkers, the CSF was also assayed for md-Tau and Aβ42. A correlation was observed between the CSF concentrations of n-APLP1 and n-CHL1 (β=0.72, P=9.1×10⁻⁴⁸) (FIG. 6A), between md-Tau and n-APLP1 (β=0.6, P=1.3×10⁻³⁰) (FIG. 6B), and between md-Tau and n-CHL1 (β=0.53, P=1.7×10⁻³³) (FIG. 6c).

Many extracellular peptides, including fragments of APP and APLP1 are known to bind extracellular amyloid plaques (Bayer et al. 1999), and therefore the group was stratified into those who, based on established CSF A1342 cutoffs, were amyloid plaque positive (Aβ42<600 pg/ml). Confirming the interpretation that amyloid plaques can confound the relationships, a much stronger relationship in amyloid-negative patients between md-Tau and n-APLP1 (β=0.85, P=3.7×10⁻¹⁰ (FIG. 6D), and between md-Tau and n-CHL1 (β=0.91, P=1.8×10⁻¹³) (FIG. 6E) was observed, whereas the relationship between n-APLP1 and n-CHL1 was found to be plaque-independent (FIG. 6F).

### Example 7—n-APLP1 and n-CHL1 are Correlated with Tau in the CSF of Healthy Controls and Elevated in MCI Patients

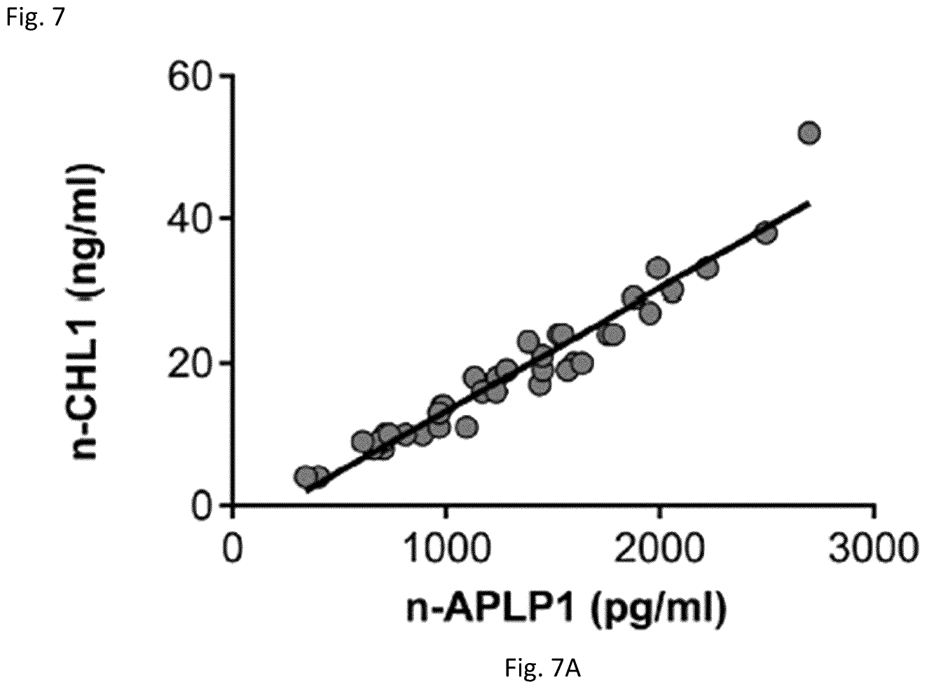

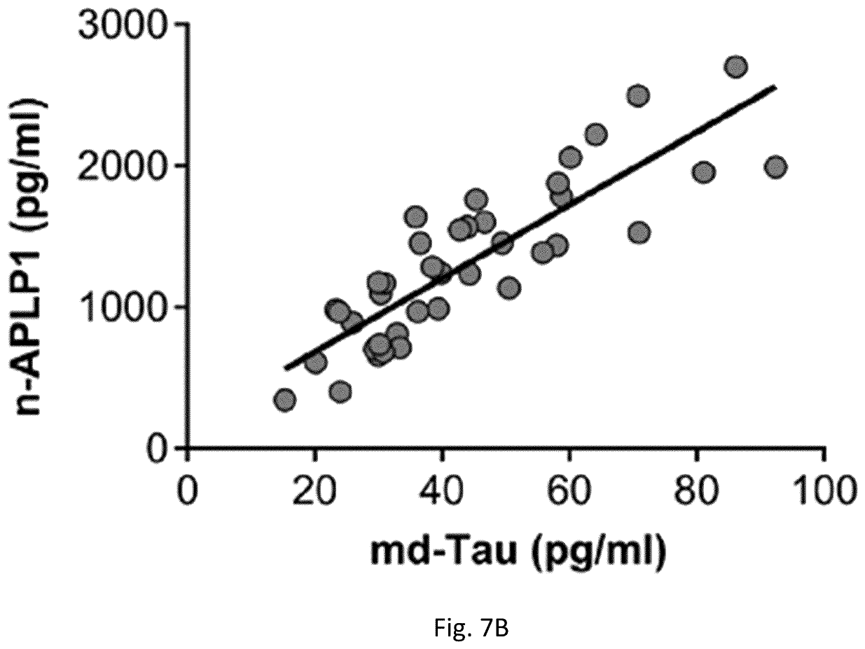

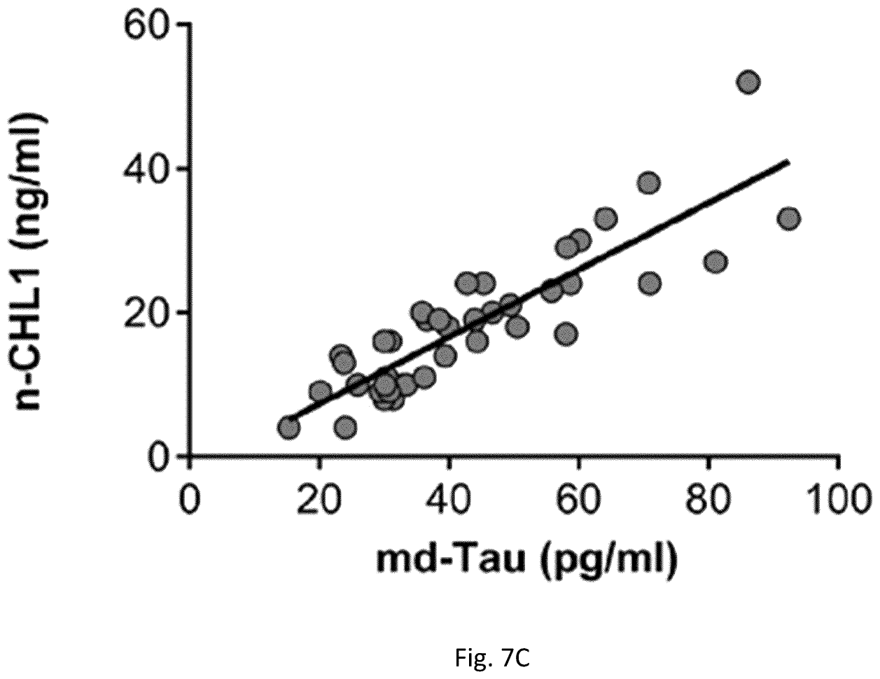

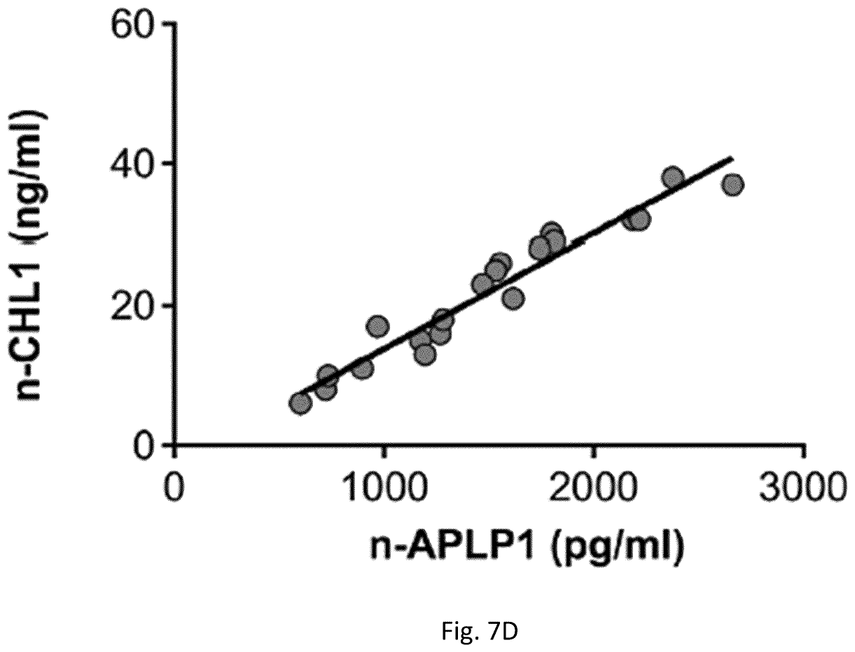

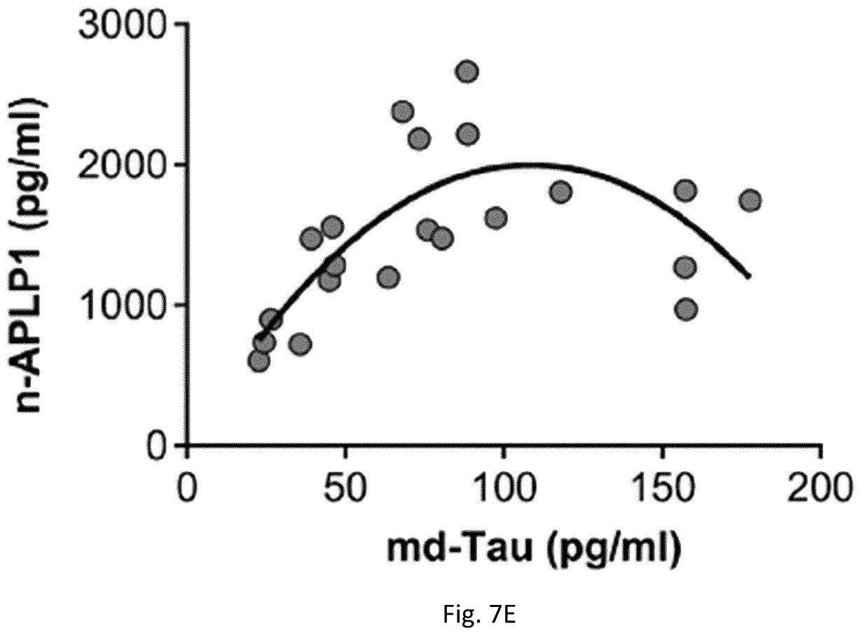

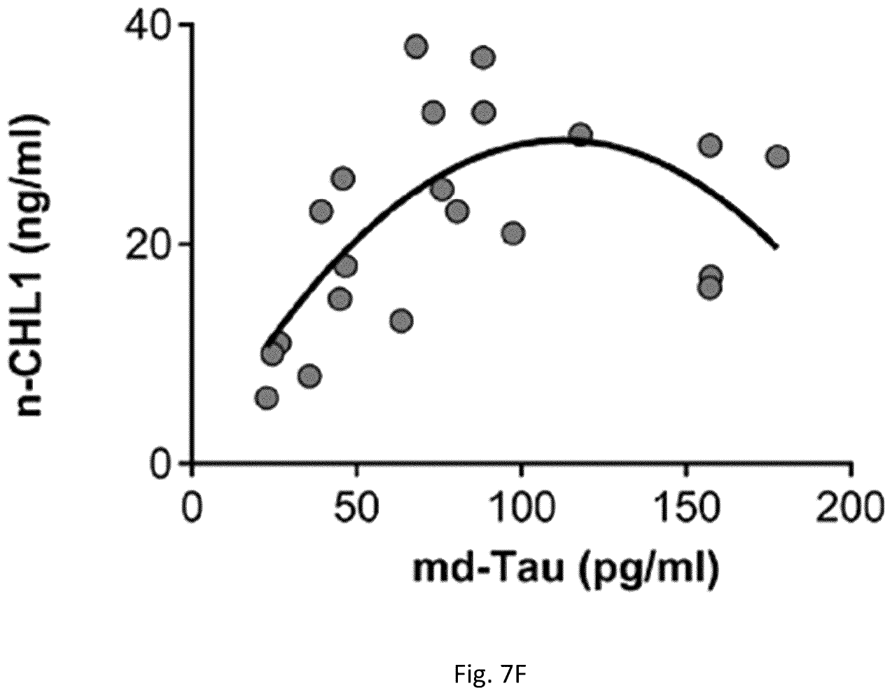

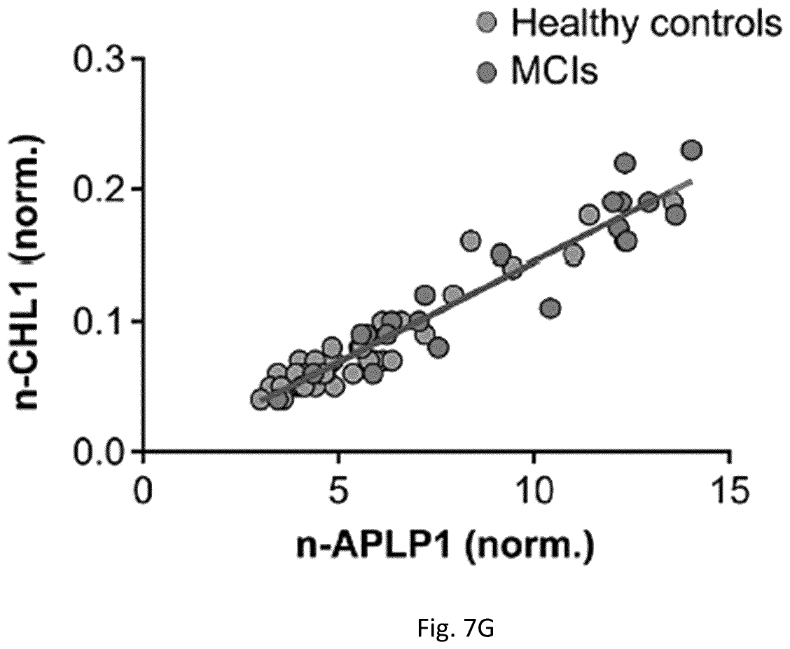

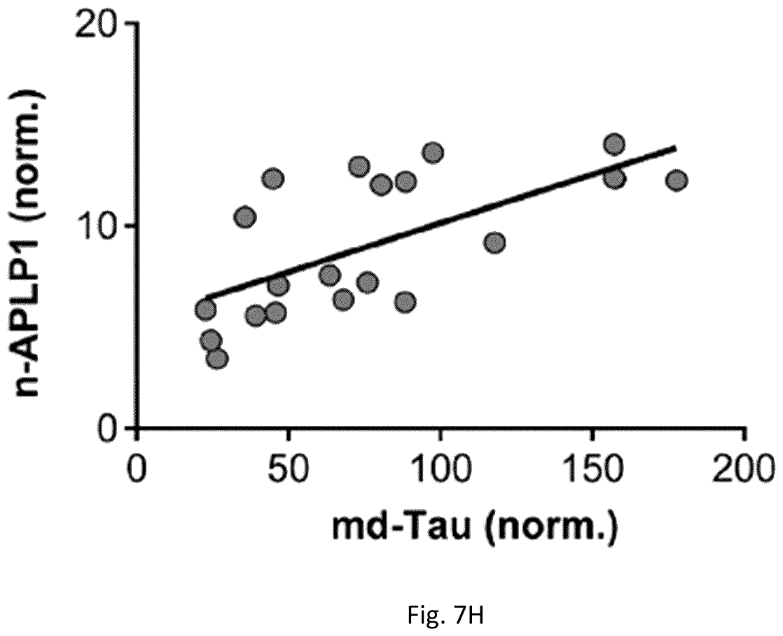

Banked CSF from 40 healthy subjects who were clinically determined to be cognitively unimpaired (mean age=68.3, range=55-87; 53% female), and CSF from 21 subjects who were clinically determined to have mild cognitive impairment (MCI) (mean age=67.0, range=52-87; 34% female) were used. Strong linear correlations in healthy controls were observed between CSF concentrations of n-APLP1 and n-CHL1 (β=0.97, P=2.4×10⁻²⁴) (FIG. 7A), md-Tau and n-APLP1 (β=0.86, P=1.7×10⁻¹²) (FIG. 7B), and between md-Tau and n-CHL1 (β=P=2.1×10⁻¹⁷) (FIG. 7C).

In MCI patients, a strong linear correlation was observed between n-APLP1 and n-CHL1 (β=0.97, P=6.5×10⁻¹³) (FIG. 7D), whereas the relationship between md-Tau and n-APLP1 (β=0.53, P=0.001) (FIG. 7E) or between md-Tau and n-CHL1 (β=0.50, P=0.002) were found to be curvilinear (FIG. 7F).

To test the assumption that this curvilinear relationship reflected the confound of amyloid plaques, when adjustment was made for CSF Aβ42, the relationship between md-Tau and n-APLP1 (β=0.67, P=0.001) or between md-Tau and n-CHL1 (β=0.79, P=0.00002) was now linear (FIGS. 7G-7I).

Next, to determine who among this cohort had evidence of AD, a tau/Aβ42 ratio cutoff of ≥0.15 was used as indicative of AD, based on a sample of 82 CSF for which this cutoff provided an optimal AUC of 0.87 with specificity (91%) and specificity (75%). The majority of subjects who were clinically unimpaired were biomarker-negative for AD and the majority of subjects who were clinically diagnosed as MCI were biomarker-positive for AD, and thus presumably represented prodromal AD (chi square=24.0, P=9.6×10⁻⁷).

To test for between-group differences, a significant elevation was found in the corrected measures of n-APLP1 (F=84.2, P=9.4×10⁻¹³) and n-CHL1 (F=78.2, P=3.2×10⁻¹²) in the prodromal AD patients vs. controls (FIGS. 8A and 8B). Of note, based on the distributions, approximately 70% of prodromal AD patients were 2 standard deviations above the means of the controls.

### Example 8—n-APLP1 and n-CHL1 are Correlated with Phosphorylated Tau in the CSF of Healthy Controls and Patients

Recent studies have reported that compared to other tau species found in the CSF, tau phosphorylation at its threonine 217 site (p-tau217) is the CSF tau ‘signature’ that is most sensitive to AD, detected in the earliest preclinical stages of the disease before the onset of neurodegeneration, and the one most specific to AD compared to other tauopathies (Barthelemy et al. 2020a; Barthelemy et al. 2020b).

Although reagents are not currently available to reliably measure mouse CSF p-tau217, the relationship of n-APLP1 and n-CHL1 to p-tau217 was examined in most of the human CSF samples. As with md-tau, it was found that p-tau217 correlates with n-APLP1 (β=0.36, P=6.5×10⁻¹¹) and n-CHL1 (β=0.37, P=6.4×10⁻¹¹) in the dementia cohort; that p-tau217 correlates with n-APLP1 (β=0.72, P=5.1×10⁻⁷) and n-CHL1 (β=0.62, P=5.0×10⁻⁶) in the healthy controls and that p-tau217 correlates with normalized n-APLP1 (β=0.63, P=0.002), and n-CHL1 (β=0.71, P=0.0003) in the MCI cohort (FIG. 9).

### Example 9— Materials and Methods for Examples 10-15

### Antibodies, Reagents and Plasmids

Antibodies were obtained from the following sources; rabbit polyclonal antibodies to VPS26b (Novus Biologicals, NBP1-92575, RRID:AB_11020053, 1:500 for Western Blot (WB), 1:200 for immunofluorescence (IF), 1:50 for Immunohistochemistry (IHC)), VPS26a (Abcam, ab23892, RRID:AB_2215043, 1:2000 for WB, 1:300 for IF), VPS26a (1:300 used for electron-microscopy (EM), a kind gift from Dr. Juan Bonifacino), neuro-tubulin (abcam, ab18207, RRID:AB_444319, 1:5000 for WB), VPS35 (Bethyl laboratories, A304-727A, RRID:AB_2620922, validated for IP, see methods below), GluA1 C-terminus (Millipore, AB1504, RRID:AB_2113602, 1:200 for IHC), mGluR5 (Alomones lab, AGC-007, RRID:AB_2039991, 1:500 for WB), Syntaxin13 (Synaptic Systems, 110 133, RRID:AB_2198225, 1:500 for WB), Rab11 (Thermo fisher, 71-5300, RRID:AB_2533987, 1:500 for WB); goat polyclonal antibodies to VPS35 (Abcam, ab10099, RRID:AB_296841, 1:300 for IF), VPS29 (Sigma, SAB2501105, RRID:AB_10602838, 1:500 for WB), EEA1 (Santa Cruz, sc-6415, RRID:AB_2096822, 1:300 for IF); mouse monoclonal antibodies to β-actin (Novus Biologicals, NB600-535, RRID:AB_2222878, 1:10000 for WB), α-tubulin (Sigma, T6074, RRID:AB_477582, 1:5000 for WB), VPS35 (Abcam, ab57632, RRID:AB_946126, 1:1000 for WB, 1:300 IF), VPS26a (Novus Biologicals, NBP2-36754, 1:200 for IF), Golgin97 (Invitrogen, clone CDF4 A-21270, RRID:AB_221447, 1:100 for IF), Syntaxin13 (Synaptic Systems, 110 132, RRID:AB_67552, 1:300 for IF), Rab? (Santa Cruz, sc-376362, RRID:AB_10987863, 1:100 for IF and 1:500 for WB), GluA1 N-terminus (Millipore, MAB2263, RRID:AB_11212678, 1:500 for WB), MAP2 (Millipore, AB5622, RRID:AB_91939, 1:400 for IHC), neuro-tubulin (Biolegends, cat #801202, RRID:AB_10063408, 1:5000 used for WB), Rab5 (Santa Cruz, SC-46692, RRID:AB_628191, 1:500 for WB); chicken polyclonal antibodies to GFAP (abcam ab4674, RRID:AB_304558, 1:500 for IHC), MAP2 (Abcam, ab5392, RRID:AB_2138153, 1:800 for IF). Secondary antibodies from LI-COR Biosciences. Dilution for IRDye 800CW (1:15000), IRDye 680RD (1:15000) and IRDye 680LT (1:20000). Alexa-conjugated fluorescent secondary antibodies were from Life Technologies (1:400 for Alexa 488 and Alexa 555; 1:300 for Alexa 647).

Reagents were of pharmacological grade, as follows: human transferrin —Alexa555 (Invitrogen, cat #T35352, lot #1716236, 1:200) and (−)-Bicuculline methiodide (Tocris, cat #2503).

Second generation lentiviral packaging plasmids VSVg and 48.9 were gifts from Peter Scheiffele (University of Basel), whereas the EGFP-tagged active, full-length Cre recombinase and catalytically inactive, truncated Cre FUGW lentiviral constructs were a gift from Thomas Sudhof (Stanford University, California). The lentiviral constructs used for the rescue experiments were purchases from OriGene, p-Lenti-C-mGFP-P2A-Puro (cat #PS100093) and p-Lenti-C-mGFP VPS26b (cat #MR204988L4). Lentiviral particles were generated using a Lentiviral Packaging kit (Origene, cat #TR30037). The VPS26a peptide used for the immunostaining (IF) validation of VPS26a antibody was from Novus Biologicals (cat #NBP2-36754PEP).

### Human Studies

### In Vivo MRI Studies

Image acquisition. The human MRI data was obtained from the Alzheimer's Disease Neuroimaging Initiative (ADNI) database. ADNI is a multicenter study aimed to develop AD biomarkers. The details of the ADNI MRI acquisition, preprocessing protocols (Jack et al., 2008) can be found in the project website. Briefly, the Tl-weighted structural MRI scans were acquired with MP-RAGE (Magnetization-prepared rapid gradient echo) sequence under 1.5T MRI scanner. A total of 188 AD patients and 169 normal subjects baseline scans were used in the current study.

The demographic information of the subjects can be found in Table 1.

TABLE 1
Demographic information of the human
subjects used in the MRI analysis

| | AD (N = 188) | Normal (N = 169) |
|---|---|---|
| Age mean (std) | 75.18 (7.50) | 75.64 (5.18) |
| Gender M/F | 99/89 | 83/86 |

Image processing and data analysis. The Tl-weighted structural images were processed using FreeSurfer, generating individual cortical surfaces and cortical thickness maps (Fischl and Dale, 2000). The individual cortical surfaces were registered into the FreeSurfer fsaverage space for inter-subject analysis. At each vertex, linear regression with cortical thickness as dependent variable was performed with AD vs. normal diagnosis as testing variable, and with gender and age as covariates. t-statistics of the regression analyses were reported as a raw t-map. The results were rendered in the flat map representation of the cortical surface using FreeSurfer utilities (Fischl et al., 1999) and custom scripts. The flat-map representation provides unbiased visualization of the whole cortex in one single view, and facilitates observation of the findings, and the identification of clustered parcellation of interest. To localize AD-vulnerable regions, we thresholded the t-map at t<−12 (passing Bonferroni correction at α=1E-22). The statistics on the surface space were converted to anatomical space using FreeSurfer utilities.

Postmortem Studies. Tissue blocks from healthy controls and AD cases were requested from the Columbia University Alzheimer's Disease Research Center (ADRC) brain bank (IRB #AAAB0192, Not Human Subjects Research under 45 CFR 46 exemption 4). Neuropathological assessment was performed using methods previously described (Vonsattel et al., 1995). All cases were rated according to Braak and Braak (Braak and Braak, 1991), Consortium to Establish a Registry for Alzheimer's Disease (CERAD) (Mirra et al., 1991), and National Institute on Aging—Reagan Institute (NIA-RI) criteria. A total of 24 cases were used in this study: healthy controls, n=16 (age ranging from 36 to 89 years old) and AD, n=8 (age ranging from 66 to 89 years old). Demographic information can be found in Table 2. Standard brain blocks3 (SBB8 samples) containing the anterior part of the TEC, where the TEC abuts the amygdala, were used for the Western blot analysis. TEC and neighboring regions harvested from each sample block (Mai et al., 2015) were homogenized using a Bel-Art Micro-Tube Homogenizer in RIPA buffer (10 times the tissue weight in mg) containing protease and phosphatase inhibitors (Roche) for the extraction of proteins. Samples were incubated for 30 min at 4° C. in rotation before centrifugation at 13,000×g for 20 min at 4° C. Protein concentration was measured using BCA assay (PIERCE) prior to Western blot analysis using the Odyssey blocking buffers (LI-COR Biosciences).

TABLE 2
Demographic information of the human subjects used in the
post-mortem studies

| | | | | Braak | | |
|---|---|---|---|---|---|---|
| | | | | Stage (NF | | |
| Subject | Sex | Age | AD | tangles) | CERAD | NIAR |
| 1 | F | 58 | NO | 0* | B | Not el. |
| 2 | F | 54 | NO | 0* | Not el. | Not el. |
| 3 | M | 36 | NO | 0* | Not el. | Not el. |
| 4 | M | 74 | NO | 0* | Not el. | Not el. |
| 5 | M | 75 | NO | 0* | Not el. | Not el. |
| 6 | M | 78 | NO | 0* | Not el. | Not el. |
| 7 | F | 89 | NO | 0* | Not el. | Not el. |
| 8 | M | 66 | NO | 0* | Not el. | Not el. |
| 9 | F | 79 | NO | 0* | Not el. | Not el. |
| 10 | F | 68 | NO | 0* | Not el. | Not el. |
| 11 | M | 85 | NO | 0* | Not el. | Not el. |
| 12 | M | 62 | NO | 0* | Not el. | Not el. |
| 13 | F | 89 | NO | 0* | Not el. | Not el. |
| 14 | F | 83 | NO | 0* | Not el. | Not el. |
| 15 | F | 83 | NO | 0* | Not el. | Not el. |
| 16 | F | 89 | NO | 0* | Not el. | Not el. |
| 17 | F | 89+ | AD | 5 | B | HIGH |
| 18 | F | 79 | AD | 5 | B | INTERMEDIATE |
| 19 | F | 80 | AD | 6 | C | HIGH |
| 20 | F | 66 | AD | 6 | D | HIGH |
| 21 | M | 81 | AD | 3 | B | INTERMEDIATE |
| 22 | F | 83 | AD | 5 | C | HIGH |
| 23 | F | 89 | AD | 5 | C | INTERMEDIATE |
| 24 | M | 86 | AD | 5 | C | HIGH |

A total of 24 cases were used in this study: healthy controls, n=16 and AD, n=8. PMI, postmortem interval; AD, Alzheimer's disease; 0*, Minimal Tau pathology consistent with normal aging; Not el., Not eligible; CERAD, Consortium to Establish a Registry for Alzheimer's Disease; NIA-RI, National Institute on Aging—Reagan Institute

### Mouse Studies

All animal procedures and experiments were performed in accordance with national guidelines (National Institutes of Health) and approved by the Institutional Animal Care and Use Committee of Columbia University, and SUNY Downstate Medical Center. Mice were maintained in groups of 5 or less with 12 h on/off light cycles. Behavioral task experiments were performed using 2 to 3 independent groups (separate cohorts) at different time point (3-4, 6-7, 12-14 and 18-months of age) during the light phase. Animals from both sexes were randomly allocated to experimental groups. All assessments were performed by someone blind to the genotype of the animals. Mice were sacrificed by cervical dislocation, and tissue samples were dissected immediately and frozen in dry ice before storage at −80C. Hippocampi were dissected from all genotypes described below and analyzed by western blot for retromer proteins.

A colony of VPS26b KO mice originally described by Kim et al., 2008 has been established. This KO line was generated by replacing exons 5 and 6 of the PS26b gene by a neomycin-resistance cassette. These animals are viable, live into adulthood, and do not display any gross physical abnormalities. VPS26a heterozygote (HET) mice were previously described and characterized in Muhammad et al. 2008. Vps26^{flox/flox} mice, containing exons 3-7 of the Vps26 gene flanked by a LoxP site in both alleles were used to generate primary neuronal cultures. This line was described in Mecozzi et al., 2014.

Lastly, mice expressing loxP-flanked VPS35 (VPS35_{flox/flox}) on a C57BL/6 background were crossed with mice expressing Cre recombinase under the Camk2α promoter to knock-out VPS35 in forebrain neurons as described in Example 1. In all experiments, littermate VPS35^{flox/flox}; Camk2α-Cre (VPS35 cKO) were compared to VPS35^{flox/flox} (VPS35 WT).

Neuronal cell culture. Hippocampal and cortical cultures were obtained from P0 mice, trypsinized for 10 mM at 37° C., dissociated by passing through a Pasteur pipette and finally plated on poly-Ornithine coated 35 or 100 mm dishes (biochemistry) or poly-Lysine-coated coverslips (Immunofluorescence). Neurons were incubated with Neurobasal media (Invitrogen) with 10% fetal bovine serum for 2-4 h before being transferred into serum-free Neurobasal-A media supplemented with B27 (Invitrogen), Glutamax, and cultured for 15 or 21 days in vitro (DIV). Media were always changed 24 h post-plating to remove any cell debris.

Cell transfection and lentivirus production. Knockout VPS26a^{-/-} or control VPS26a^{flox/flox} primary hippocampal neurons were generated by infecting cells at DIV 5, for a period of 10 days, with a lentivirus carrying either the catalytically active Cre or catalytically dead Cre recombinase (ΔCre, control), respectively. Both Cre recombinases encoded two nuclear localization sequences (NLS) and were fused to eGFP containing a third NLS for enhanced nuclear targeting. Lentiviruses were produced in HEK-293T cells. Briefly, virus-containing medium was collected 72 h after transfection, centrifuged at 2,800 g for 5 mM, filtered through a 0.45 mm filter (Millipore), supplemented with 10 μg/ml polybrene (Sigma-Aldrich) and applied at a 1:5 ratio to hippocampal/cortical medium. The KD efficiency was further monitored by western blot analysis.

Western blotting. Mouse tissue samples were homogenized using a Glas-Col homogenizer in an ice-cold lysis buffer consisting of 20 mM Tris HCl (pH 7.4), 1% TritonX, mM NaCl and protease and phosphatase inhibitors (Roche) for the extraction of proteins. After centrifugation at 17,000 g for 10 mM at 4° C., supernatants were collected and the protein concentration was measured using BCA assay (PIERCE) prior to Western blot analysis using the Odyssey blocking buffers (LI-COR Biosciences). For all other immunoblots, hippocampal neurons were lysed for 10 mM at 4° C. in RIPA buffer with protease and phosphatase inhibitor cocktails, centrifuged for 15 mM at 13,000 g, and supernatant were processed for SDS—PAGE and immunoblotting. The images were acquired with the Odyssey Infrared Imaging System (LI-COR Biosciences) and analyzed by the software program Image Studio Lite Ver 5.0 as specified in the Odyssey software manual.

Pulse—chase Transferrin. Primary hippocampal cultures were starved in warmed Neurobasal-A medium containing 1% HEPES (Invitrogen) for 30 mM and incubated with 15 μg/ml of human transferrin —Alexa 555 for 30 mM at 37° C. Medium was then replaced and transferrin was chased for 10 min (to load recycling endosomes) before cell fixation.

Glycine stimulation. Glycine stimulation experiments for immunofluorescence analysis of VPS26 co-stainings with different intracellular markers were performed in DIV 15 primary hippocampal neurons, essentially as described by Park et al. (2006). Very briefly, neurons were incubated at 37° C. for 5 mM in extracellular solution (ECS) containing 124 mM NaCl, 2 mM CaCl2, 3 mM KCl, 10 mM HEPES (Invitrogen) and 10 mM Glucose (Sigma) (pH 7.4). Activity was induced with E4 supplemented with 20 μM bicuculine and 200 μM Glycine (Sigma) for 5 min, and finally in E4 with 20 μM bicuculine for 5 mM before fixation and imaging. For transferrin stainings, a transferrin uptake was performed right before glycine treatment. Transferrin was only added to the first step of the glycine treatment (5 mM incubation with E4). Neuronal activity was induced in absence of transferrin.

Co-immunoprecipitation (Co-IP). Co-immunoprecipitation experiments were performed using 500-600 μg of proteins extracted from primary neuronal cultures using a lysis buffer containing 0.5% Triton X-100, 50 mM Tris, 100 mM EDTA, 150 mM NaCl, pH 7.3. Protein extracts were incubated overnight with 10 μg of anti-VPS35 (Bethyl laboratories, A304-727A), anti-VPS26a (Abcam, ab23892) and anti-VPS26b (Novus biologicals, NBP1-92575) antibodies pre-incubated with protein-G beads (Invitrogen). Precipitates were subsequently washed five times with IP buffer and boiled for 30 mM at 50° C. in 100 ul of reducing SDS sample buffer 2X, followed by 5 min at 90° C. Immunoprecipitates were fractionated by SDS-PAGE and immunoblotted using same antibodies as probes. Input samples represent 10 μg of total protein corresponding to 2% of total protein used for each co-immunoprecipitation.

Cell surface biotinylation. Surface biotinylation pulldowns were performed according to 1 the manufacturer's instructions (Pierce). Briefly, cortical neurons were washed twice with PBS and “live” labeled with 1 mg/ml of Sulfo-NHS—SS-Biotin at 4° C. for 30 min. After being rapidly washed 3 times with PBS, cells were quenched with 100 mM of glycine for 10 min. Cell surface biotinylated proteins were pulled down overnight, at 4° C., from cell lysates by streptavidin precipitation and detected by western blotting analysis. Lysates not subjected to streptavidin beads were analyzed as a measure of total cellular protein levels.

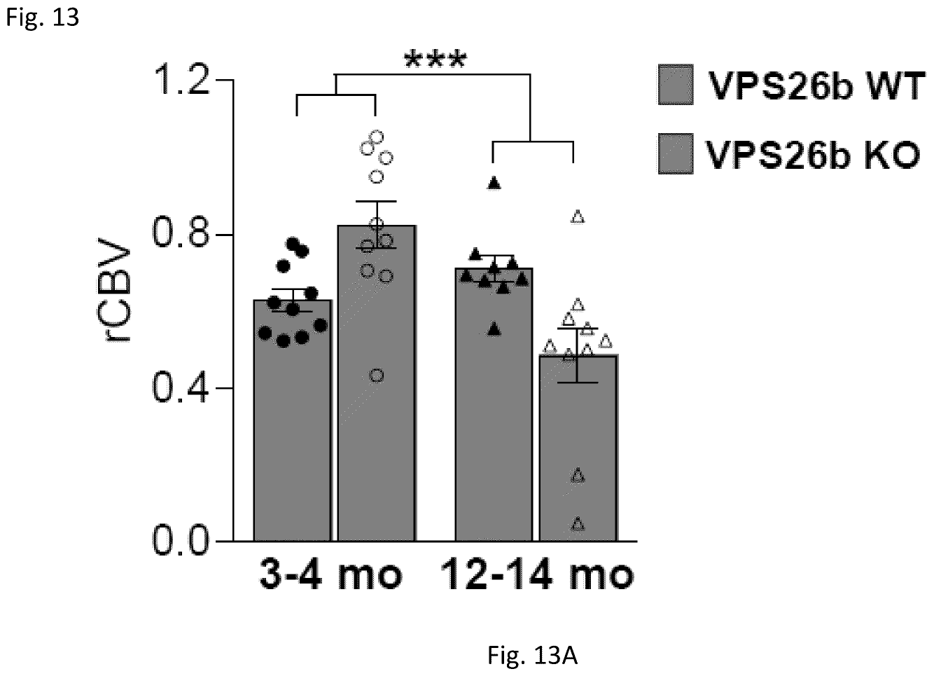

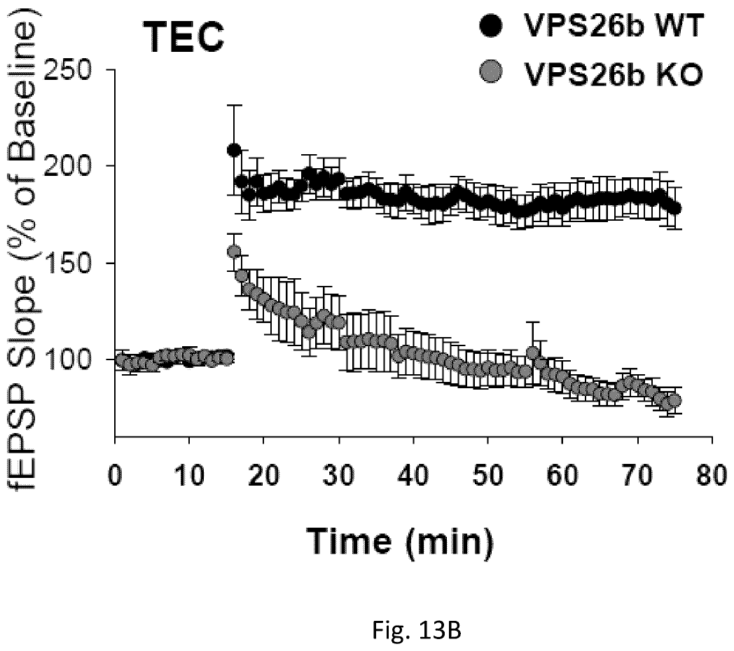

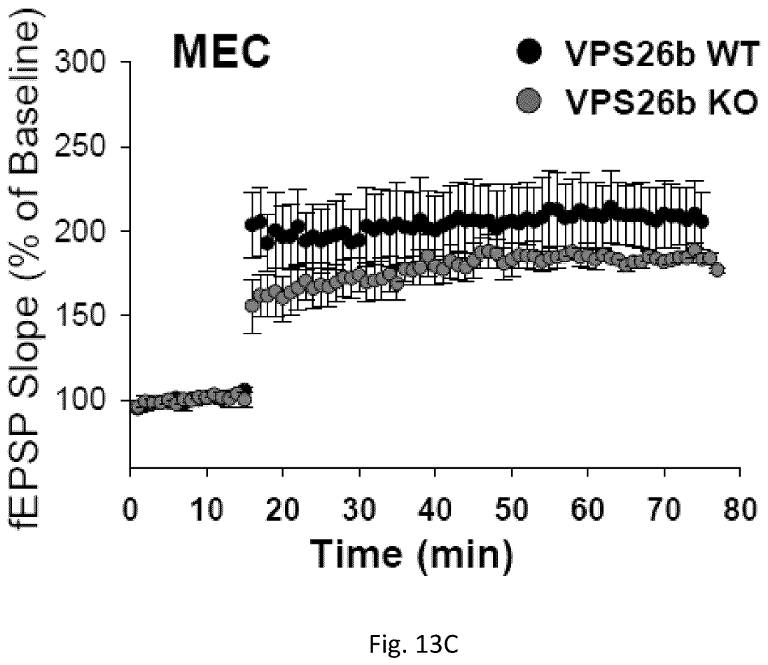

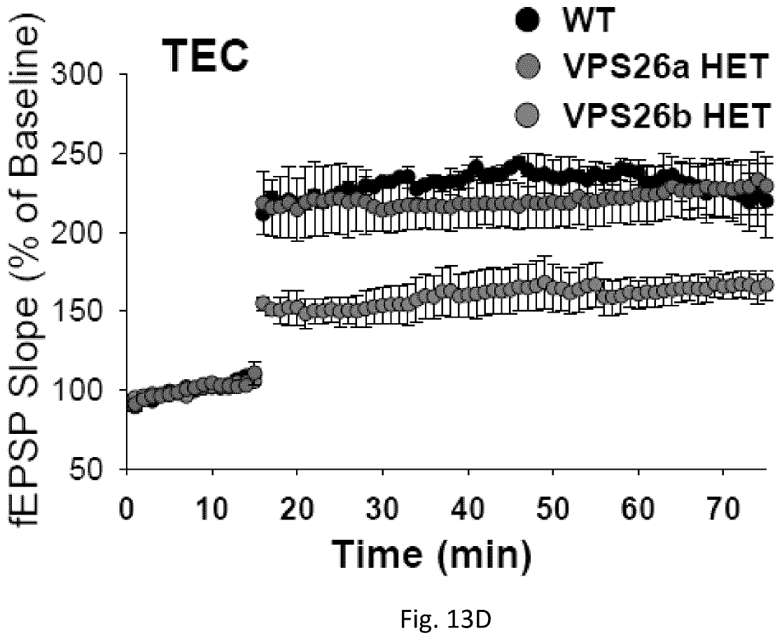

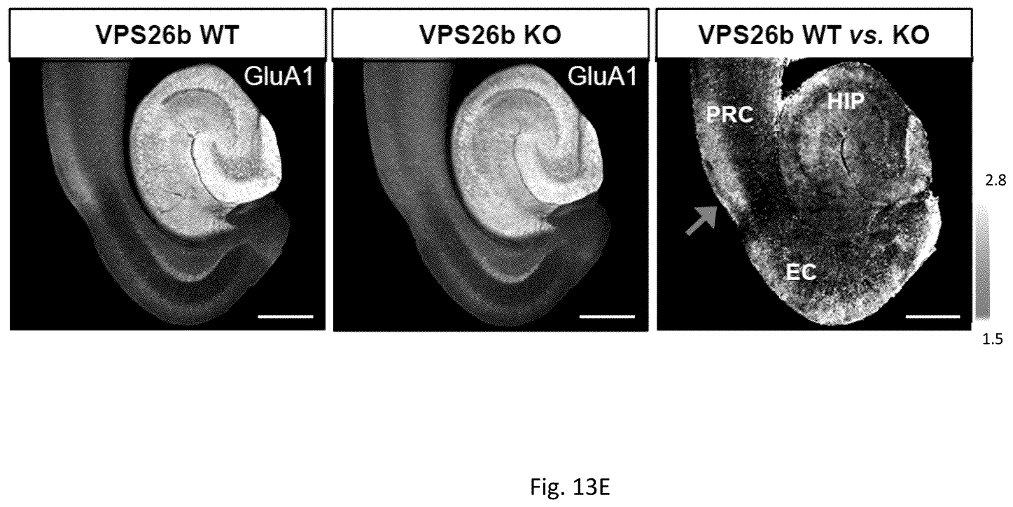

Confocal microscopy. Primary hippocampal neurons cultured on coverslips were fixed in a final concentration of 2% paraformaldehyde (PFA) and 0.5% sucrose (mixed with culture medium) and permeabilized with 0.05% saponin in PBS supplemented with 5% donkey serum. For all intracellular stainings, neurons were incubated overnight with primary antibody and further incubated for 45 min with secondary antibodies (Molecular probes). Images were captured with a Zeiss LSM 700 META confocal microscope equipped with a 63× Plan-Apochromat objective and HeNEi, HeNe₂ and argon lasers. Colocalization analysis (% colocalization) was obtained based on Pearson's correlation, that is, (Pearson's coefficient) 2×100, using the JACoP Plugin on ImageJ (Bolte and Cordelieres, 2006). Z-stack images of soma and proximal dendrites (with no thresholds applied), were used for data analysis shown in all Figures, except for FIG. 13B. For this latter, regions of interest (ROI's) were used to investigate the intracellular localization of GluA1 in VPS26b KO and WT cultures. Three to four proximal dendritic segments of 40 μm were analyzed and averaged for each cell. Cell surface GluA1 staining intensity was determined using the ‘Multi Measure’ build-in option in ImageJ and the intensity of the staining was normalized to the cell area. For staining, hippocampal neurons were initially “live” incubated with an anti-N-terminal GluA1 antibody (MAB2263; 1:300) diluted in Neurobasal-A media containing 1% HEPES for 30 min at 37° C. After a brief washing in prewarmed Neurobasal A, cells were fixed with 4% PFA. Fixed nonpermeabilized neurons were further incubated with a fluorescent secondary antibody for 1 hour at RT. When required, following a short fixation with 2% PFA, cells were permeabilized using PBS-0.1% Triton X-100 for 10 min, and further incubated with MAP2 antibody for the detection of dendrites.

Confocal microscopy GluA1 pixel-based analysis. Confocal microscopy of GluA1 stainings shown in FIG. 13F were co-registered into a group-wise template space using an inverse-inconsistent diffeomorphic co-registration algorithm, as described previously (Sabuncu et al., 2009, Avants et al., 2011, Khan et al., 2014). The intensity of the GluA1 staining was normalized to the mean intensity of each brain section. Normalized GluA1 intensity was analyzed using pixel-based analysis with two sample test implemented in SPM8 (Wellcome Department of Imaging Neuroscience).

Immunohistochemistry analysis. Mice were anesthetized, rinsed by cardiac perfusion with 0.9% saline buffer and further fixed with 4% PFA. Whole brains were excised and post fixed in 4% PFA overnight, followed by cryoprotection treatment in 30% sucrose (wt/vol) in PBS for 16 h. Tissue blocks were then embedded in Optimal Cutting Temperature (OCT) compound and stored at −80° C. Briefly, 30 μm free-floating horizontal sections were washed three times with PBS and incubated at 4° C. overnight on a rotator in 1 ml of primary antibody diluted in PBS containing 0.3% Triton X-100 (vol/vol) and 5% normal serum (vol/vol). After three washes with PBS-T (0.1% Triton X-100), the sections were incubated for 30 min with secondary antibodies (Molecular probes). Following three washes with PBS-T, and one wash with PBS, sections were mounted on slides. Fluorescent images were collected using a Zeiss LSM 700 META confocal microscope equipped with a 63×Plan-Apochromat objective and HeNEi, HeNe₂ and argon lasers.

Ultracryomicrotomy and Immunogold Labeling. For ultrathin cryosectioning and immunogold labeling, hippocampal neurons (15 DIV) were fixed with a mixture of 2% (wt/vol) PFA and 0.125% (wt/vol) glutaraldehyde in 0.1 M phosphate buffer (PB), pH 7.4.Cell pellets were washed with PB, embedded in 10% (wt/vol) gelatin and infused in 2.3 M sucrose. Mounted gelatin blocks were frozen in liquid nitrogen and ultrathin sections were prepared with an EM UC6 μltracryomicrotome (Leica). Ultrathin cryosections were collected with 2% (vol/vol) methylcellulose, 2.3 M sucrose and single or double immunogold labeled with antibodies and protein A coupled to −10 or 15-nm gold (PAG10 and PAG15), as previously reported (Truschel et al., 2009). Sections were observed under a Philips CM-12 electron microscope (FEI; Eindhoven, The Netherlands) and photographed with a Gatan (4k×2.7k) digital camera (Gatan, Inc., Pleasanton, CA). The relative distribution of VPS26b and VPS26b in hippocampal cultures was evaluated by EM analysis of randomly selected cell profiles from two distinct grids. Approximately 100 gold particles for each condition were counted and assigned to the compartment over which they were located. The definition of the distinct compartments was based on their morphology and their previous characterization by immunogold labeling with different organelle markers. Results are presented as a percentage of the total number of gold particles in each compartment and is represented as mean±S.E.M. A quantitation of non-specific background labeling was performed for VPS26a and VPS26b. Background labeling accounted for less than 2.1% of the total number of gold particles for VPS26b, and to less than 1% for VPS26a labeling.

### Behavioral Analysis

Object Context Recognition (OCR) task. The OCR task was carried out as previously described (Wilson et al., 2013) with minor modifications. Briefly, mice were handled daily for a week prior to the study and then given two habituation sessions. In both sessions they were individually exposed to two distinct environments (context X and context Y) for 5 min. Testing began 1-2 days after the last habituation session. In the OCR task, mice receive two test sessions separated by 24 hrs. Each session is divided into four sample phases and a test phase, each lasting 3 min. In the sample phases two identical objects (either A1:A2 or B1:B2) are placed in far corners of a context (context X or Y). A 2-min interval separate the four sample phases: phases 1 and 4 comprised objects A1 and A2 in context X, phases 2 and 3 comprised objects B1 and B2 in context Y. There is a 5-min interval between the end of sample phase 4 and the test phase. In the test phase, the animal is tested in either context X or Y with objects A3 and B3. Thus, during the test phase one object is in the same environment as in the sample phase, and the other object is in the different environment to the sample phase. In a second session (24 hrs later) the order of the sample phases are reversed and the test environment changes for each individual mouse. Thus, in sample phases 1 and 4, a mouse that had previously been exposed to A1:A2 in context X is now exposed to B1:B2 in context Y, and in phases 2 and 3 it is exposed to A1:A2 in context X. This tests the mouse's memory for object context associations as one of the objects will have been experienced in this context before, while the other will not. Test object, test context, order of context presentation in the sample phases and side of presentation was counterbalanced across mice. All training and testing sessions were recorded using the automated ANY-maze video tracking software. Discrimination between the objects were calculated using a discrimination ratio, calculated by dividing the mean time exploring the novel object by the mean of the total time exploring the novel and familiar objects during the test session.

This value was multiplied by 100 to obtain a percentage preference for the novel object (Tnovel/lTnovel+Tfamiliarl×100).

Novel Object Recognition (NOR). The NOR task was performed as previously described (Barker and Warburton, 2011). Very briefly, the day prior to training, mice were habituated to experimental apparatus consisting of a white rectangular open field (60 cm×50 cm×26 cm) for 10 min in the absence of any objects. On the second day, mice were placed in the experimental apparatus in the presence of two identical objects and allowed to explore them for 10 min. After a retention interval of 24 h, mice were placed again in the apparatus, where one of the objects was replaced by a novel object. Exploration of the objects was defined as the mice facing and sniffing the objects within 2-cm distance and/or touching them. Sitting and turning around on the object were not considered exploratory behavior. All training and testing sessions were recorded using the automated ANY-maze video tracking software. The ability of the mouse to recognize the novel object was determined by dividing the mean time exploring the novel object by the mean of the total time exploring the novel and familiar objects during the test session. This value was multiplied by 100 to obtain a percentage preference for the novel object (Tnovel/lTnovel+Tfamiliarl×100). The positions of the objects in the test phase and the objects used as novel or familiar were counterbalanced between the animals.

Modified Radial Arm Water Maze (RAWM). The 2-days RAWM task was carried out as previously described (Alamed et al., 2006). Briefly, the behavioral apparatus consisted of a pool 1.2 m in diameter filled with water made opaque with white paint. Dividers were placed into the pool to generate a contiguous space with six equidistantly spaced arms radiating from the center. A 10-cm escape platform was at the end of a designated goal arm, which remained fixed for each mouse throughout the experiment. Spatial cues were present on the walls of the testing room. On each trial, the mouse started the task from a different randomly chosen arm. Each trial lasts 1 min Entry into an arm with no platform or failure to select an arm after 10 sec was scored as an error. Animals that enter an incorrect arm were guided back to the start arm. At the end of each trial, animals were allowed to rest on the platform for 20 s. Each mouse was tested for 15 trials each day for two consecutive days. On the first day, mice were trained for 15 trials, with the first 12 trials alternating between visible (flagged) and hidden (submerged) platform. The last 3 trials of the first day and all of the 15 trials of the second day were performed with a submerged platform. Results were analyzed by dividing the 30 trials into 10 trial blocks and calculating the average error for each trial block.

Visible platform test. Mice that were subject to the 2-days RAWM were also tested on a visible platform to rule out impairments of vision, motivation, motor coordination, as well as cognitive deficits that are not restricted to spatial learning. Briefly, behavioral tests were carried out in the same pool but without arms and with a visible (flagged) platform. The platform location was randomly modified to eliminate any contribution of external spatial cues. Mice were given two sessions of three trials each day over 2 days. Each animal was allowed to swim for 1 min from a random location. The time taken (latency) and speed to reach the platform were recorded and analyzed using a ceiling-mounted camera, anHVS-2020 video tracking system and the EthoVision software. Failure to reach the platform was scored as 60 s. Results were analyzed by dividing the 12 trials into 4 trial blocks and calculating an average value for each trial block.

### MRI Analysis

Image acquisition. Cerebral blood volume (CBV)-fMRI was used to image two independent groups of mice, young and old male VPS26b KO mice and their WT littermates, with the imaging protocol as previously described (Khan et al., 2014). A Bruker BioSpec 94/20 (field strength, 9.4 T; bore size, 20 cm) horizontal small animal MRI scanner with software ParaVision 6.0.1 (Bruker BioSpin, Billerica, MA, USA) and a 23-mm 1H circularly polarized transmit/receive capable mouse head volume coil were used for the imaging. Mice were anesthetized using the medical air and isoflurane (3% volume for induction, 1.1-1.5% for maintenance at 1 liter/min air flow, via a nose cone). A flowing water heating pad was used to maintain the body temperature at around 37° C. Sterile eye lubricant was applied after each scan. T2-weighted images were acquired before and 36 min after intraperitoneal injections of the contrast agent Gadodiamide (Omniscan; GE Healthcare, Princeton, NJ, USA) at the dosage of 10 mmol/kg. T2-weighted images were acquired with a fast-spin echo acquisition (repetition time, 2,500 ms; effective echo time, 45 ms; rapid acquisition and relaxation enhancement factor, 16; in plane resolution, 60 μm; slice thickness, 250 μm).

Generating CBV maps. As previously described (Khan et al., 2014), CBV was mapped according to changes in the transverse relaxation time (ΔR2) induced by gadolinium injection. CBV was derived by normalizing ΔR2 to the mean ΔR2 signal present in the posterior cerebral artery (SCA), as delineated by a blinded rater.

Voxel-based analysis. MR images were first skull stripped to yield whole-brain volumes using an automatic rodent brain extraction algorithm based on 3-D pulse-coupled neural networks (PCNN), as described previously (Chou et al., 2011). Mice whole-brain volumes processing was conducted using custom Linux bash and Matlab scripts. Volumes were co-registered into a group-wise template space using an inverse-inconsistent diffeomorphic co-registration algorithm, as described previously (Sabuncu et al., 2009, Avants et al., 2011, Khan et al., 2014). CBV maps were analyzed 968 using voxel-based analysis with a general linear model implemented in SPM8 (Wellcome Department of Imaging Neuroscience). Data were modeled in a factorial framework with genotype and age included as the between-subjects factors. The genotype X age interaction effect with the direction showing age-related CBV worsening defects in KO mice was contrasted with a t-test. Result was corrected for multiple comparisons using Monte Carlo simulation implemented in AFNI-AlphaSim.

### Ex Vivo Analyses

Electrophysiology. Hippocampal horizontal slices were obtained as previously described (Cepeda-Prado et al., 2012). In short, mice were anesthetized with ketamine/xylazine, brains removed and placed in modified artificial cerebral spinal fluid (aCSF) bubbled with O₂ and CO₂ (pH 7.4) and sectioned through the ventral hippocampus into 400 μm-thick slices. Recordings were performed at an approximate position A/P−3.8 mm, M/L 3.0 mm, and D/V −5.6 mm (based on the C57Bl/6J mouse brain atlas, http://www.mbl.org/atlas/atlas.php). The horizontal brain slices contained all the regions of the hippocampal formation: DG, CA3, CA1, Sub, pre/para Sub, LEC, MEC, and perirhinal cortices (slice had an orientation that was similar to the middle hippocampus MRI slice used). Two synapses were tested, TECII to TECIII and MECII to MECUM The slice field excitatory post synaptic potentials (fEPSP) were recorded in the TEC layer III and MEC layer I/II with glass electrodes filled with NaCl 150 mM (2-3 MΩ resistance), which were elicited by stimulating TEC layer II and MEC layer II using a tungsten bipolar electrode. Input-output relationship curves were obtained and a stimulus evoking ˜40-50% of the maximum response was selected for the rest of the experiment. For the long term potentiation (LTP) experiments a stimulus as described above was used, a baseline of test responses was obtained (15 mM with an inter-stimulus interval of 30 s). LTP was induced on the TEC synapse by high frequency stimulation (HFS, 3 trains of 100 pulses at 100 Hz, 10 seconds interval) as previously described (Criscuolo et al., 2015) and on the MEC synapse by a high frequency stimulation of 2 trains of 100 pulses at 100 Hz, 60 second interval (modified from Ma et al., 2008). Responses were recorded for 60 mins after HFS. The field-EPSP (fEPSP) slope was measured and expressed as percentage of baseline. The results are expressed as mean±S.E.M. To verify glutamatergic synaptic transmission AMPA-receptors and NMDA-receptors were blocked with bath applied NBQX (16 μM) and 2-APV (10 μM), respectively. At the end of the experiment, which abolished fEPSP.

### Statistical Methods

Data are expressed as geometric mean with error bars representing±SEM. Data normality was analyzed using a D'Agostino-Pearson omnibus normality test. A two-way ANOVA with Bonferroni's post hoc test was used for the OCR and NOR behavioral tasks (three independent age groups). For the comparison of VPS26 HET mice performance in the OCR task, unpaired student's t-test or non-parametric Mann-Whitney T-test was used as described in figure legend. To compare the escape latency, swim speed and performance in the 2-days RAWM, a two-way repeated—measures ANOVA with Bonferroni post-tests was used. Unpaired student's t-test was used for some of the biochemical experiments and immunofluorescence analysis using two-tailed distribution with 1013 equal variance (P<Unpaired t-test with Welch's correction was performed for groups with unequal variances as stated in the figure legend. Differences between more than two groups were evaluated by one-way ANOVA with post hoc Tukey's multiple comparisons test. All the statistical analysis described above were performed using the GraphPad Prism version 7.00 for Windows (GraphPAd Software). Mice and human MRI statistical analysis was performed using SPM8 (Wellcome Department of Imaging Neuroscience), while the electrophysiology data and human WB analysis were analyzed using SPSS. The “n” indicated on figure legends represent either the number of animals or cells analyzed per condition or group.

### Example 10—VPS26b is Highly Expressed in Brain Tissue and Primary Neurons

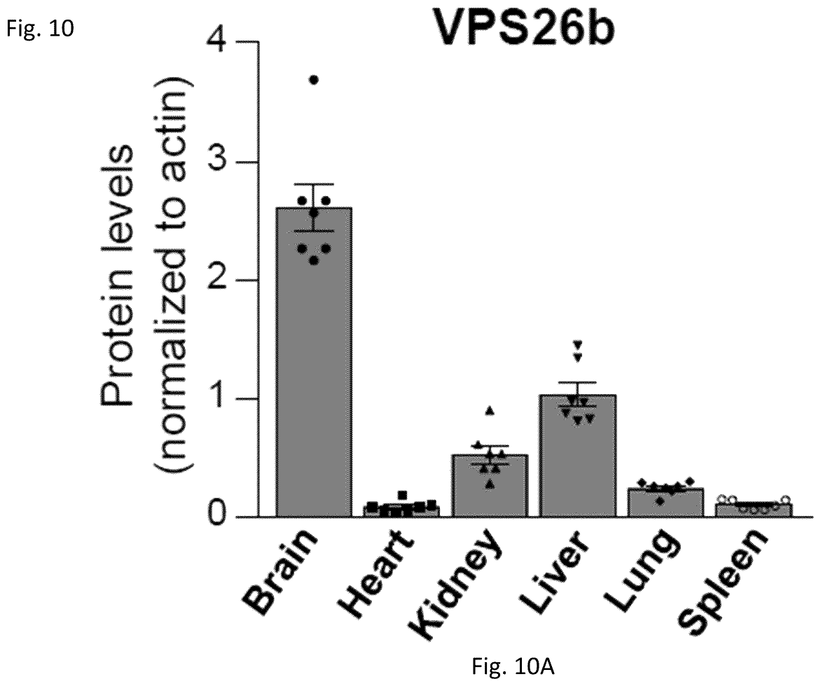

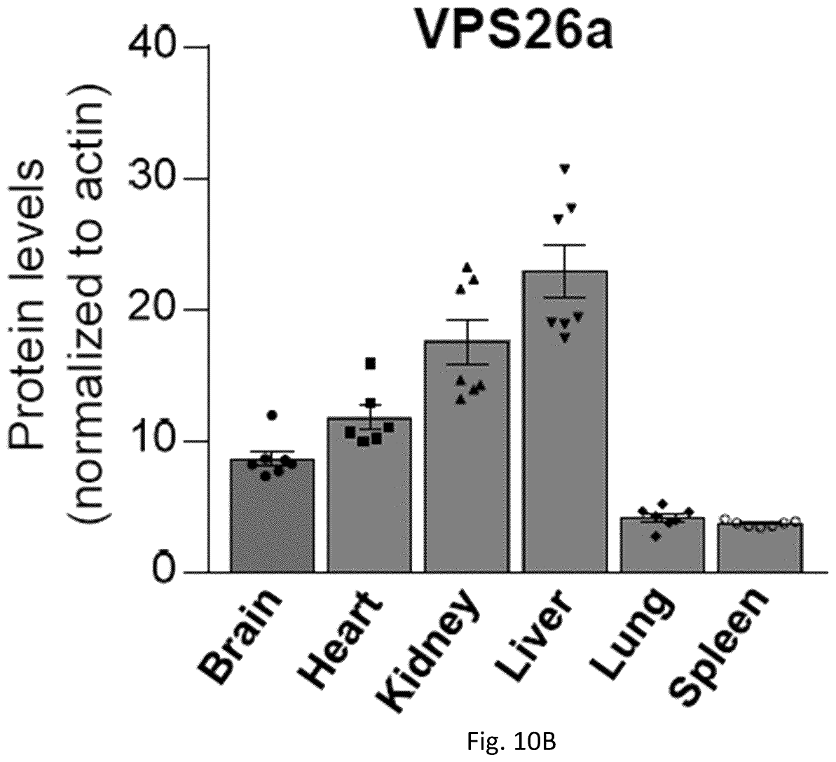

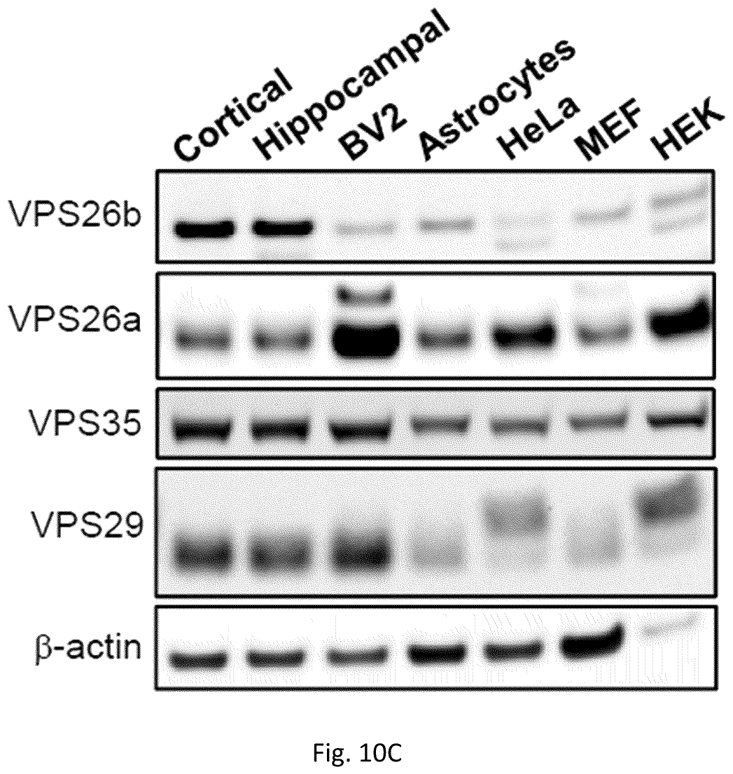

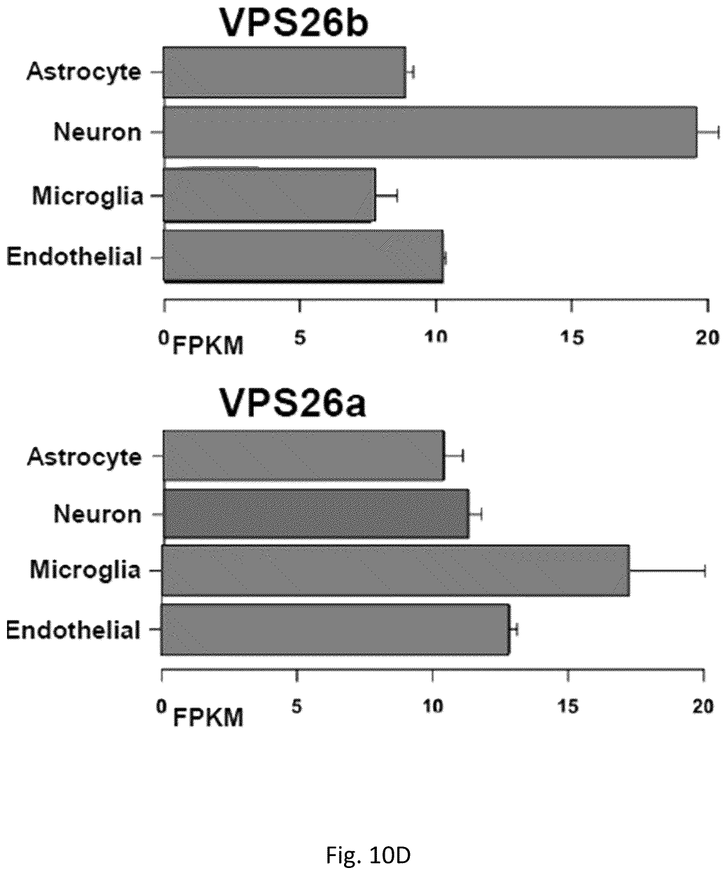

Different organs from wild-type (WT) mice were harvested and analyzed by Western blot analysis. This analysis confirmed previous findings showing that, compared to VPS26a, VPS26b is enriched in the brain and in neuronal cells (FIGS. 10A and 10B). A search of an mRNA database (Zhang et al., 2014) agreed with and extended the protein level analysis, showing that VPS26b is highly expressed in neurons, compared to astrocytes and other brain cell types, while the expression profile of VPS26a is more distributed across brain cells (FIG. 10D) Immunofluorescence analyses in fixed hippocampal slices was performed, and found that VPS26b is present in neurons, as labeled with MAP2, but not in GFAP-positive astrocytes (results not shown). These results suggested that VPS26b is differentially expressed in the brain where it is enriched in neurons.

### Example 11—VPS26b and VPS26a Define Distinct Retromer Cores

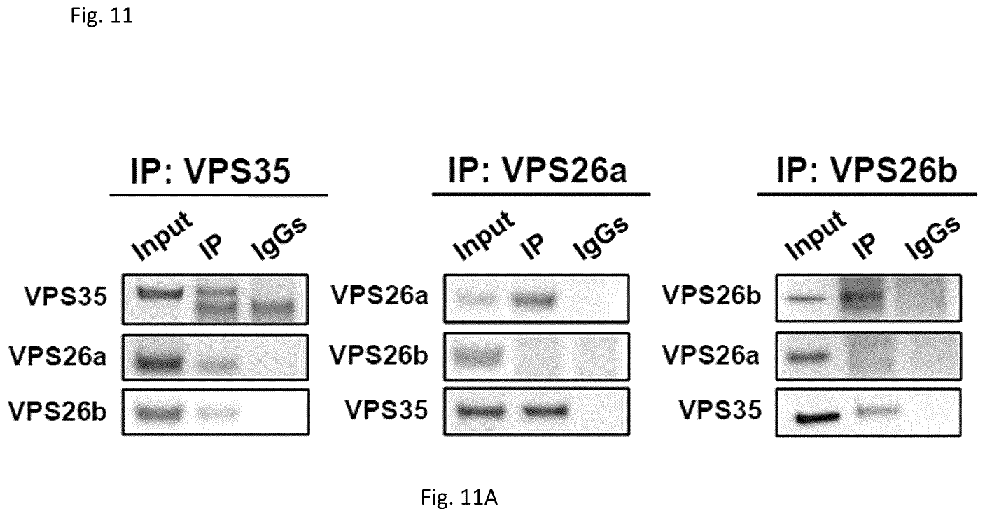

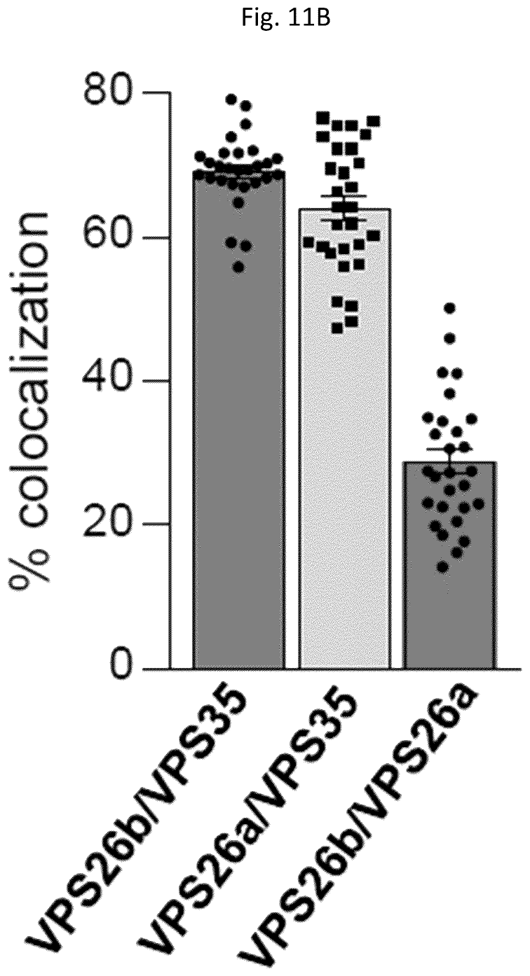

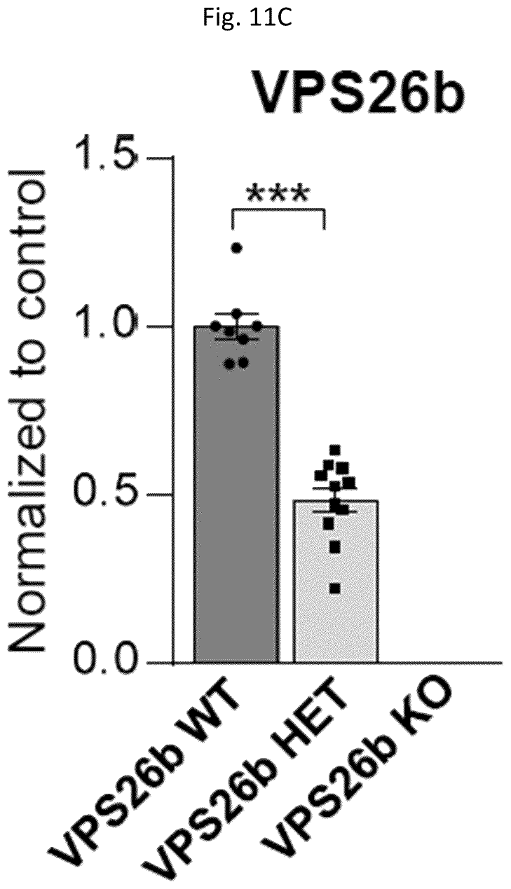

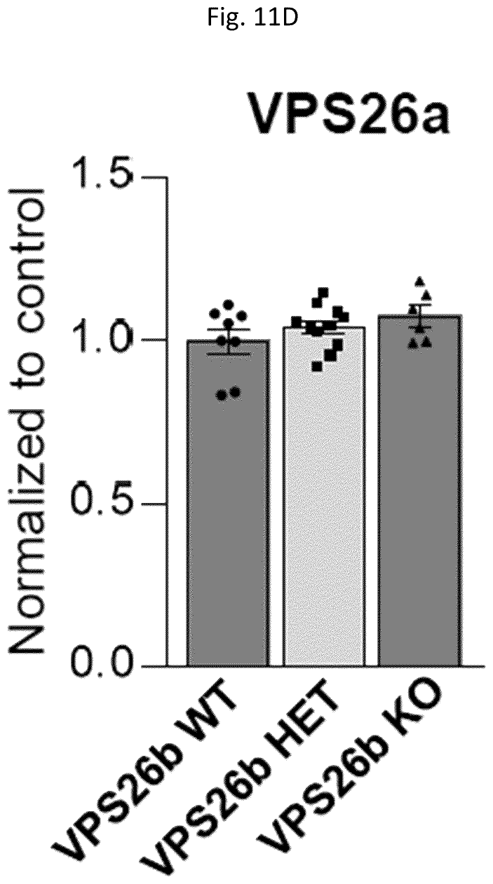

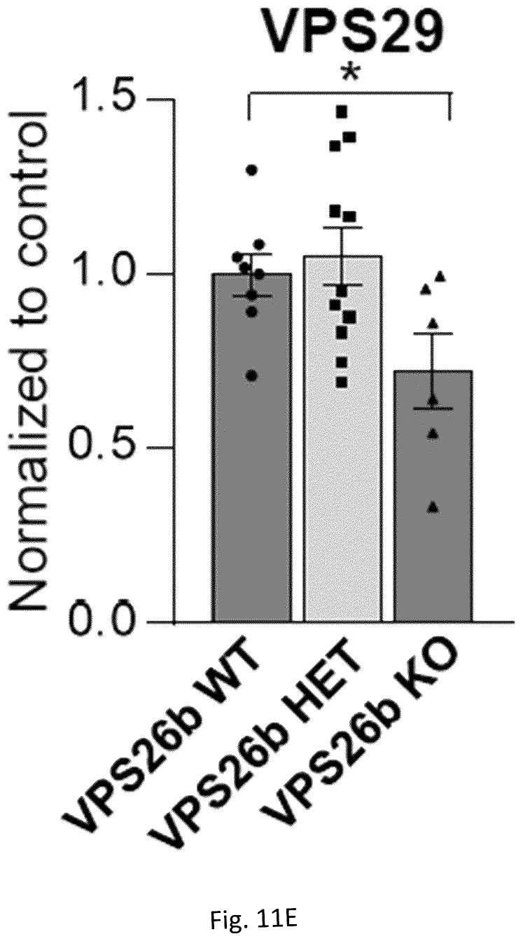

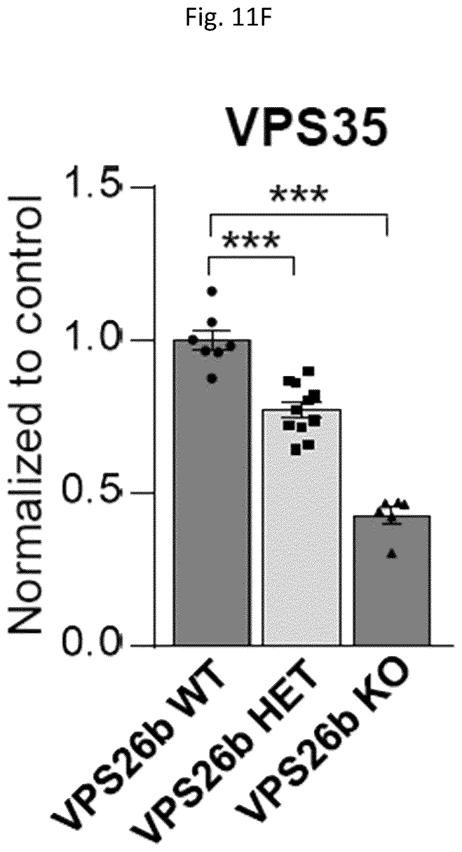

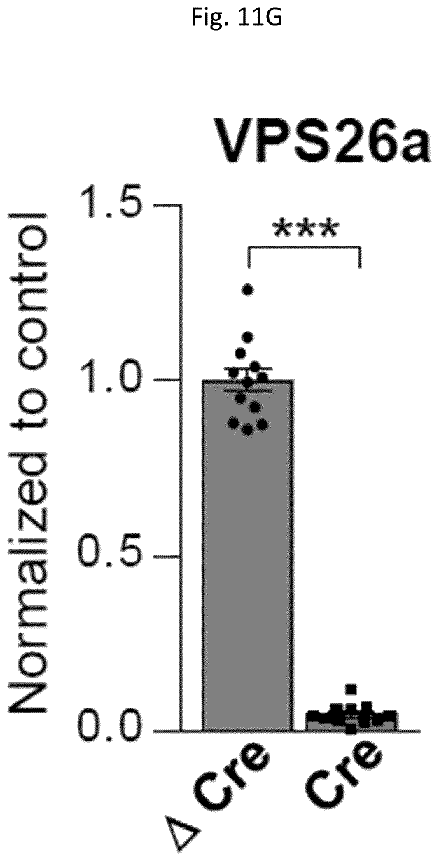

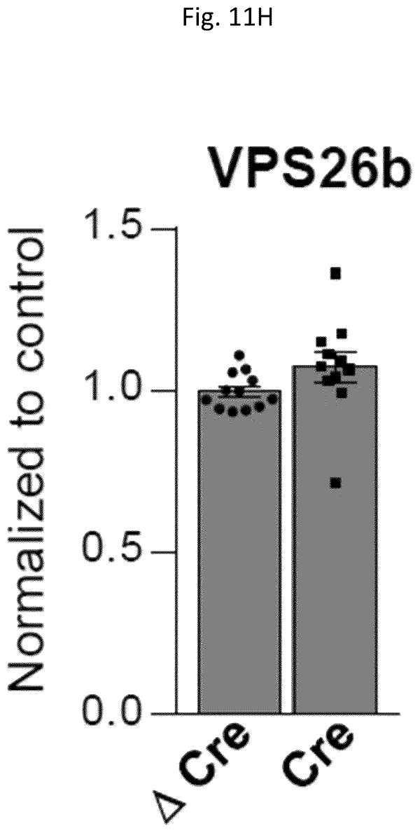

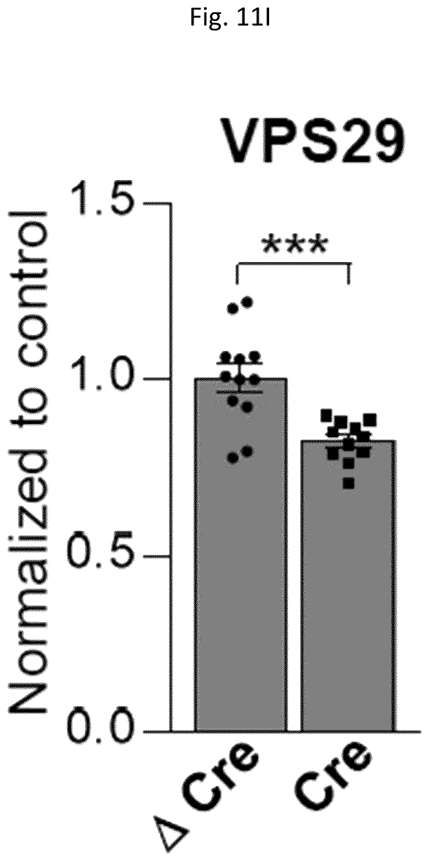

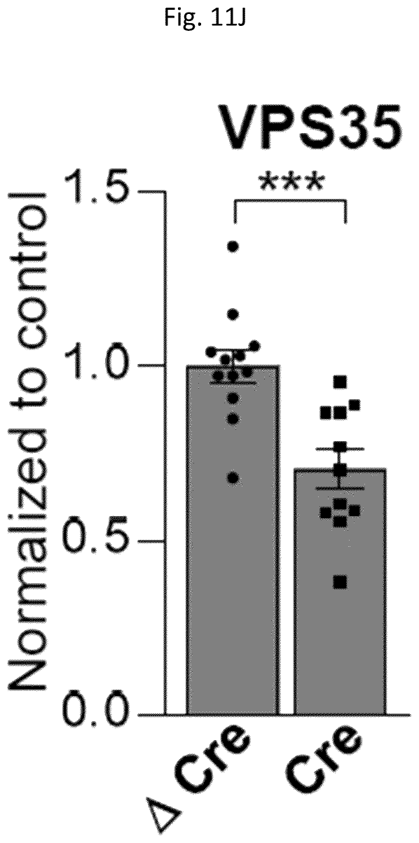

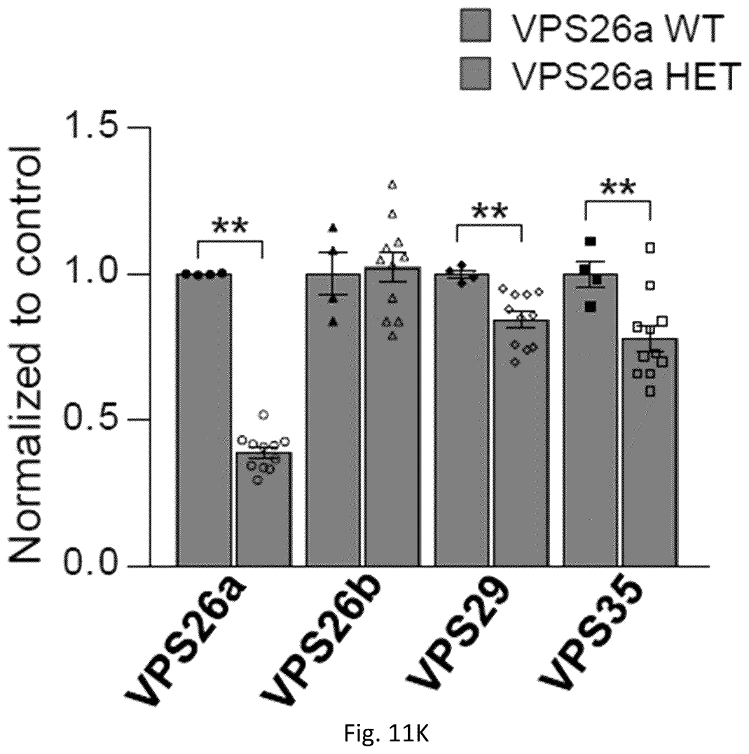

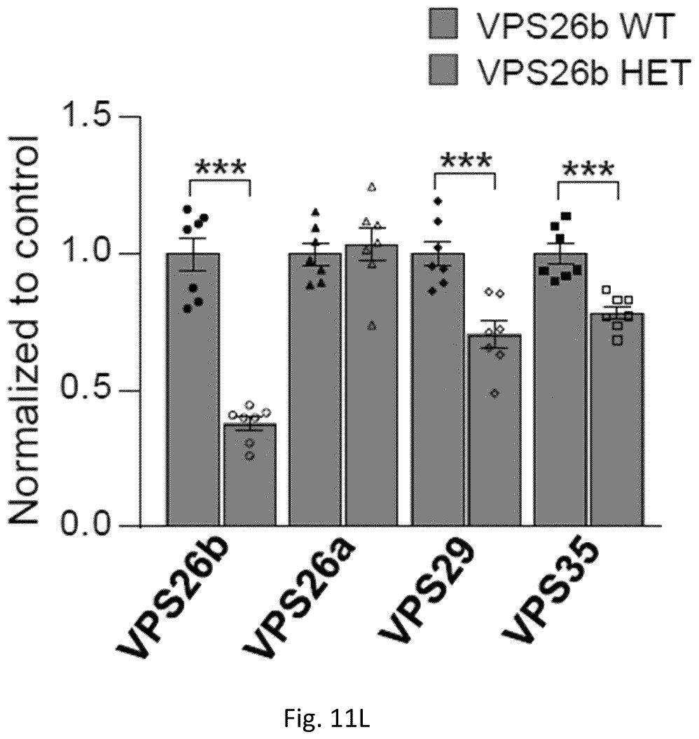

The two VPS26 paralogs that exist in neurons might be interchangeable in binding a single retromer core, or they might be part of two distinct cores. To begin addressing this question, co-immunoprecipitation experiments were performed in primary hippocampal neurons, showing that while VPS35 pulled down both paralogs, VPS26a pulled down VPS35 but not VPS26b, and VPS26b pulled down VPS35 but not VPS26a (FIG. 11A). Providing further evidence for separate cores, confocal microscopy showed that while VPS35 highly colocalized with VPS26b and with VPS26a, both paralogs exhibited low intracellular colocalization (FIG. 11B).

These observations suggested that neurons might have two separate retromer cores. To further test this hypothesis, previous studies showing that a primary knock down of one core member causes a secondary reduction in other bound members were relied on (as reviewed in (Small and Petsko, 2015)). If VPS26a and VPS26b are part of separate cores, a primary reduction in one should not affect the levels of the other. By examining primary neuronal cultures derived from VPS26b KO mice, a secondary reduction was found in VPS35 and VPS29, but no effect on VPS26a (FIGS. 11C-11F). To overcome the problem of VPS26a KO lethality, a VPS26α^{flox/flox} mouse model was used, and infected cultured neurons derived from these mice, with a lentivirus expressing either Cre recombinase (Cre), or a catalytically dead version of Cre (ΔCre). A near complete depletion of VPS26a had no effect on VPS26b (FIGS. 11G-11J). Further supporting these findings, fluorescent microscopy showed that VPS26b-VPS35 colocalization was unaffected in VPS26a depleted neurons, and that VPS26a-VPS35 colocalization was unchanged in VPS26b KO neurons (data not shown).

Next to validate these findings in vivo, three genetically engineered retromer-deficient mouse models were used (FIGS. 11K and 11L). Since homozygote VPS26a^{-/-} mice are embryonic lethal, the expression levels of both VPS26a and VPS26b in heterozygote mice were compared. The hippocampus from 6 month-old mice heterozygote VPS26a (Muhammad et al., 2008) (VPS26a HET) and their WT littermates (VPS26a WT), or mice heterozygote for VPS26b (Kim et al., 2010) (VPS626b HET) and their WT littermates (VPS26b WT) were harvested, and analysis performed by Western blot. It was found that a primary deficiency in VPS26a caused a secondary reduction in VPS35 and VPS29, but had no effect on VPS26b (FIG. 11K). Conversely, a primary deficiency in VPS26b caused a secondary reduction in VPS35 and VPS29, but had no effect on VPS26a (FIG. 11L).

The protein expression levels of both VPS26 paralogs in VPS35 deficient mice were investigated. As VPS35 KO mice are embryonic lethal, a novel mouse model in which the CamKIIα-Cre/LoxP system was used to knock out VPS35 in forebrain neurons. By harvesting the hippocampus of 6 month-old VPS35 cKO mice and their WT littermates (VPS35 WT), Western blot analysis showed that a primary deficiency in VPS35 caused a secondary reduction in VPS29 and both VPS26 paralogs (results not shown).

Collectively, these studies supported the hypothesis that, within the brain, primary neurons are enriched with a distinct retromer core, VPS29-VPS35-VPS26b.

### Example 12—VPS26b Redistributes to Recycling Endosomes During Neuronal Stimulation

Based on the studies above, and because previous studies have suggested that the VPS26 paralogs might serve different functions in endosomal trafficking (Bugarcic et al., 2011), neuronal cell cultures were used to test the hypothesis that VPS26b and VPS26a might differentially localize to endosomal compartments with which retromer has been associated— the early endosome, the recycling endosome, and the late endosome.

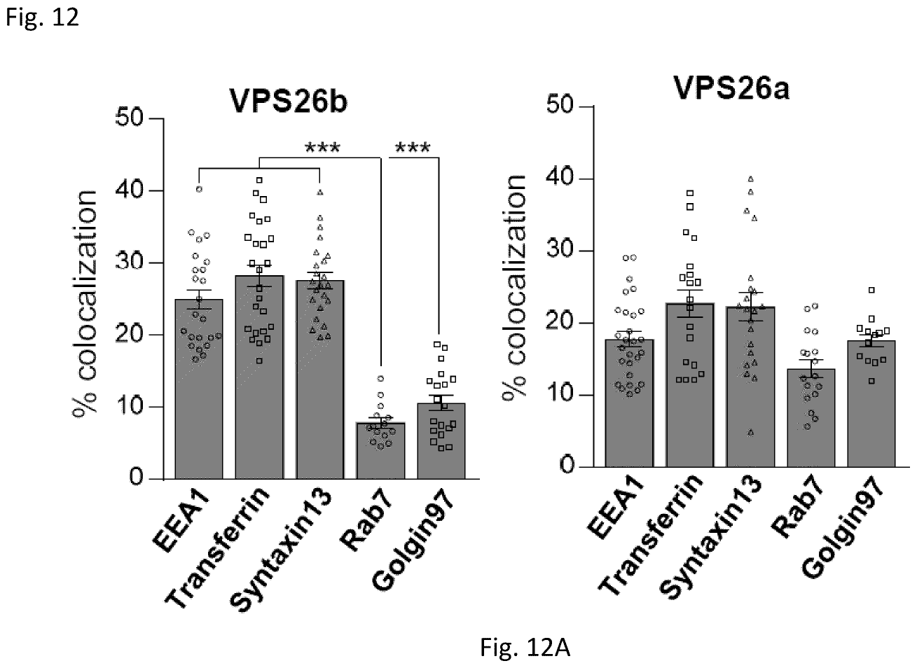

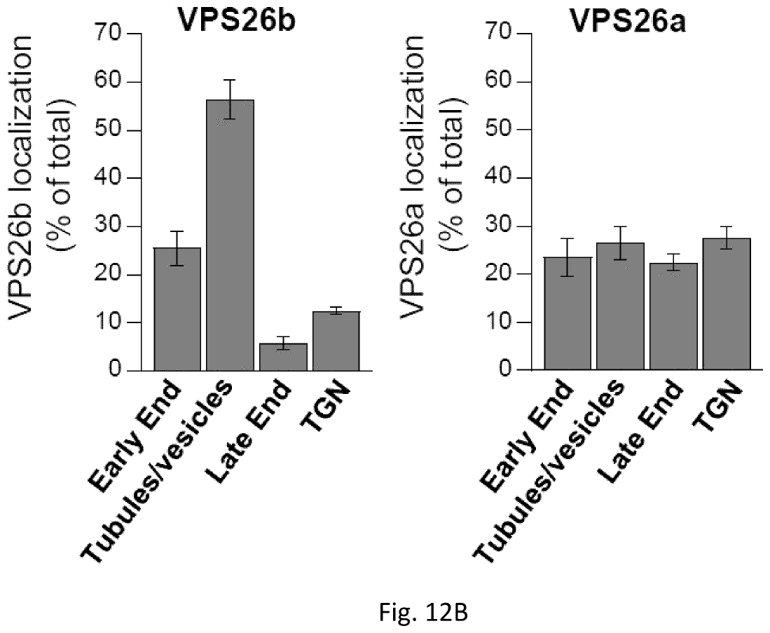

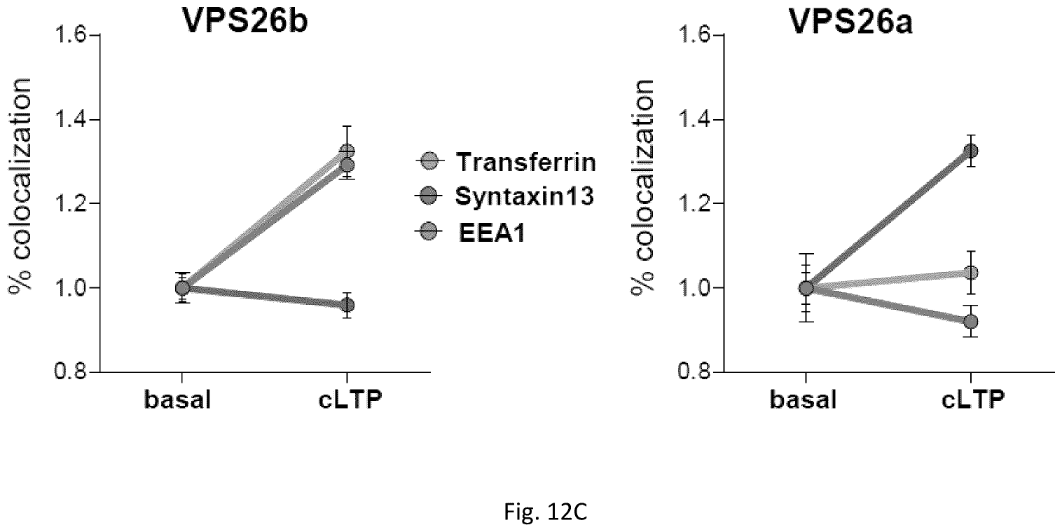

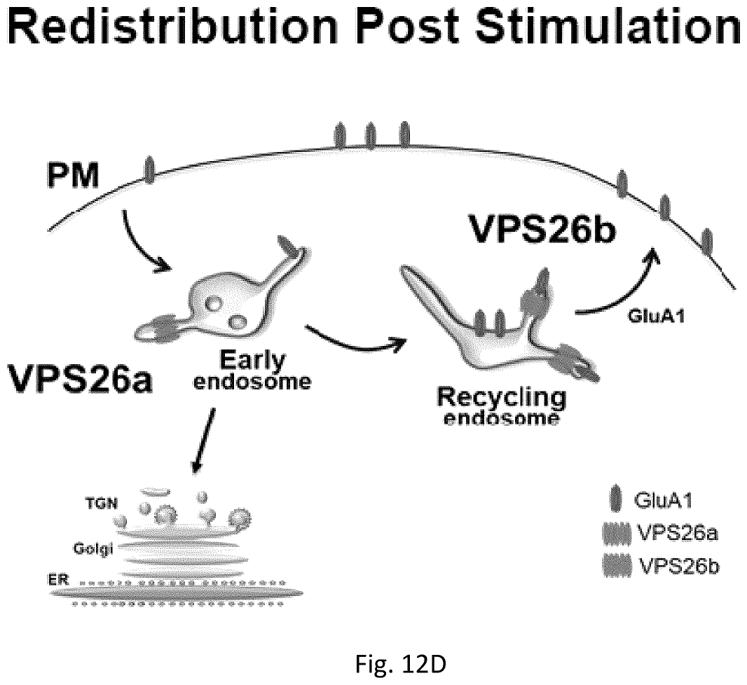

To begin addressing this question, confocal microscopy and markers of various organelles were used to compare the endogenous subcellular localization of the two VPS26 paralogs (FIG. 12A). While overlaps in localization were noted, significant differences were detected. VPS26a was found broadly distributed across all membrane compartments—the early (EEA1) and recycling endosomes (Pulse-chase transferrin and Syntaxin13), but also the late endosome (Rab7) and Trans-Golgi network (Golgin97). Compared to VPS26a, VPS26b localized more to early and recycling endosomes Immuno-electron microscopy (IEM) analysis performed in hippocampal cultures further revealed an enrichment of VPS26b in tubulo-vesicular structures most likely derived from the recycling pathway (FIG. 12B).

Since retromer is required for LTP, which is known to redistribute proteins to endosomal compartments in which they function, it was tested whether there would be redistribution of the two VPS26 paralogs after chemical LTP (cLTP) (FIG. 12C). Upon cLTP stimulation, the paralogs showed different patterns of redistribution. While VPS26b increased its localization to recycling endosomes, but not to early endosomes, VPS26a increased its localization to early endosomes but not to recycling endosomes (FIG. 12D).

Collectively, these results showed that compared to VPS26a, VPS26b differentially localizes to recycling endosomes, particularly upon neuronal stimulation.

### Example 13—the Trans-Entorhinal Cortex Differentially Depends on VPS26b

Since VPS26b KO mice live into adulthood, it was sought to determine whether there are brain regions differentially sensitive to VPS26b depletion. First an fMRI variant was used that maps regional levels of cerebral blood volume (CBV), a neuroimaging indicator of basal metabolism (Small et al., 2011) with particularly high spatial resolution, and thus useful for interrogating the whole rodent brain in localizing metabolic defects (Khan et al., 2014). Young (3-4 months) and old (12-14 months) VPS26b KO mice and their VPS26b WT littermates were imaged and tested for a genotype X age interaction. Across the whole brain, voxel-based analysis reliably identified a focal age-related defect in the VPS26b KO mice. This metabolic defect was found to localize to the borderzone between the perirhinal and the entorhinal cortices, which in the clinical literature is called the trans-entorhinal cortex (TEC). A region-of-interest analysis was used to document that the interaction was driven by age-related dysfunction in the TEC of the VPS26b KO mice (FIG. 13A).

To validate this strikingly focal effect, electrophysiological analysis was performed in acute slices of the medial temporal lobe, whose slice location and orientation were generated and guided by the neuroimaging study. Results revealed a dramatic defect in LTP in the TEC of VPS26b KO mice (FIG. 13B), with normal basal synaptic transmission. Confirming the anatomical selectivity of the effect, while visual inspection revealed a mild reduction, no statistically reliable deficits were observed in the MEC of VPS26b KO mice (FIG. 13C). Confirming the paralog selectivity of the effect, a significant LTP defect was observed in the TEC of VPS26b heterozygote mice, but no defects were observed in VPS26a heterozygote mice (FIG. 13D).

The dramatic LTP defects observed in VPS26b deficiency is consistent with a reduction in the AMPA-sensitive glutamate receptor GluA1, a glutamate receptor known to be recycled by retromer (Temkin et al., 2017). Accordingly, brain slices of the medial temporal lobe were stained for GluA1, whose slice location and orientation were guided by the neuroimaging study. Across all regions analyzed, the most reliable GluA1 reduction was observed in the TEC of VPS26b KO mice (FIG. 13E), precisely in the same location where fMRI identified metabolic dysfunction and electrophysiology identified LTP defects. Collectively, these observations show that VPS26b depletion specifically targets the TEC region, the subregion of the brain most vulnerable to AD (Khan et al., 2014).

### Example 14—Cognitive Profiling Supports VPS26b's Regional Association

There are no established behavioral tasks whose performance selectively depends on the TEC. Instead, since the TEC is at the borderzone of the perirhinal cortex and lateral entorhinal cortex, we used available tasks whose performance localize to these two parahippocampal regions: The novel object recognition (NOR) task for the perirhinal cortex (Barker and Warburton, 2011), and the object-context recognition (OCR) task for the lateral entorhinal cortex (Wilson et al., 2013). In addition, as the hippocampus is interconnected with the paraphippocampus, we also used the modified 2-days radial arm water maze (2d-RAWM) to evaluate hippocampal function (Alamed et al., 2006).

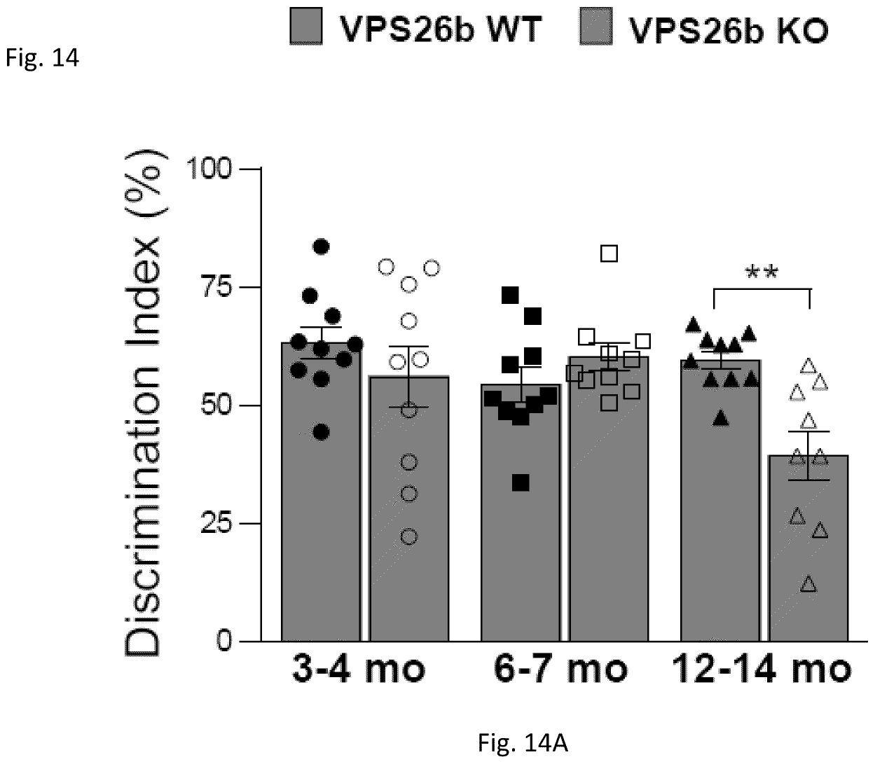

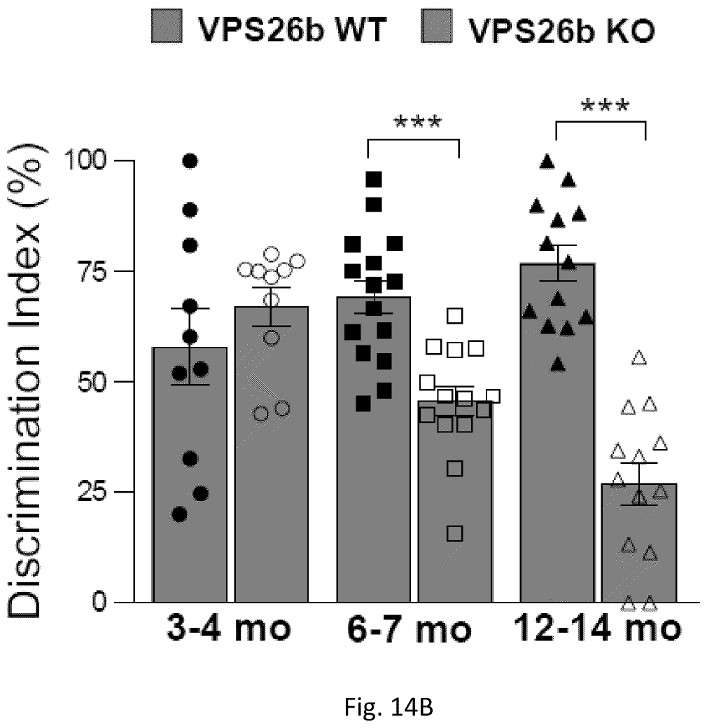

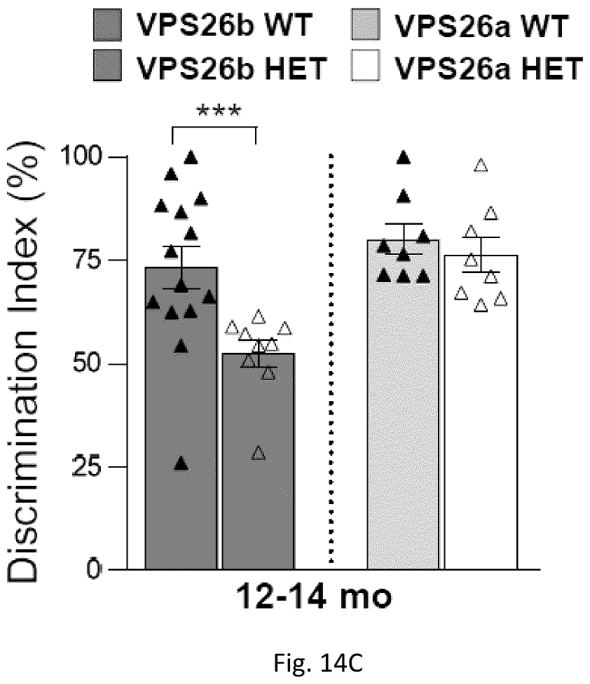

These tasks were used to cognitively profile young (3-4 months), middle-aged (6-7 months) and old (12-14 months) VPS26b KO mice and their WT littermates (FIG. 14). While at the oldest ages some defects were observed in both the NOR and the OCR tasks, results showed that OCR performance was most sensitive to VPS26b depletion, affected first and foremost (FIGS. 14A and 14B). More specifically, a significant genotype X age interaction was observed, driven by an age-dependent worsening beginning at middle age. No defects on the RAWM task were observed at this age, and only a trend towards a defect was observed at older ages (results not shown).

Since VPS26a KOs are embryonic lethal, the paralog specificity of this effect was confirmed by finding that VPS26b heterozygote, but not VPS26a heterozygote mice, have OCR defects (FIG. 13C). Collectively, this cognitive profile supports the conclusion that, compared to VPS26a, the general vicinity of the TEC differentially depends on VPS26b.

### Example 15—VPS26b Mediates Glutamate-Receptor Trafficking from the Recycling Endosome

Previous studies have established that recycling endosomes are the intracellular reservoirs of GluA1 and that GluA1 recycling to the cell surface is retromer-dependent (Temkin et al., 2017). These studies, however, used VPS35 depletion to establish this retromer-dependence, and, as shown above, VPS35 causes secondary reduction in both VPS26a and VPS26b. The findings reported above, suggest that it is the VPS26b-retromer that is more likely to play a role in GluA1 recycling.

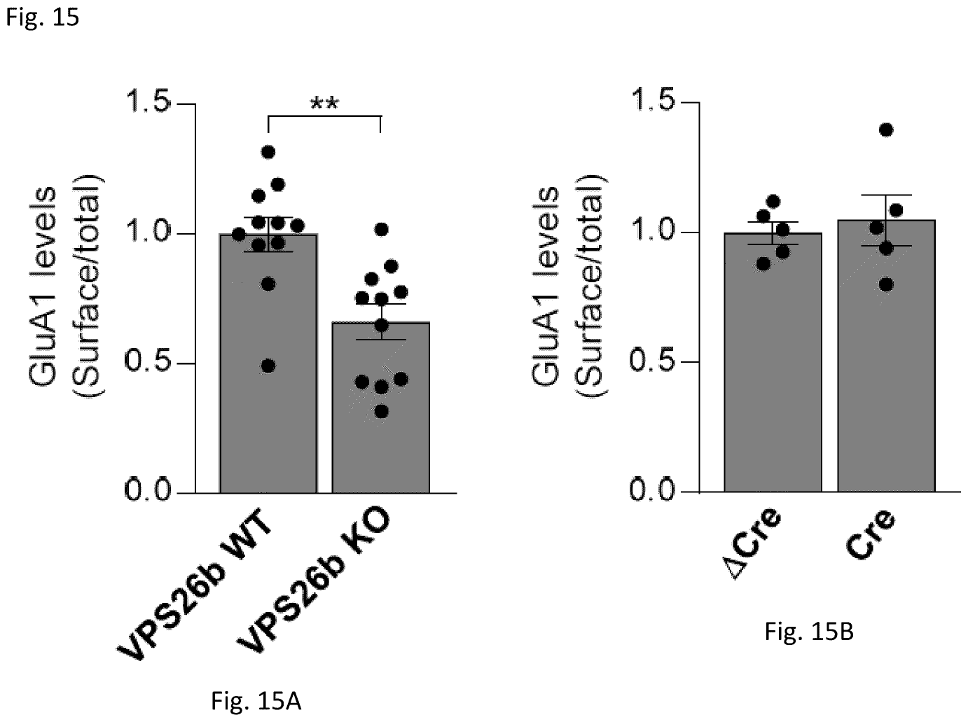

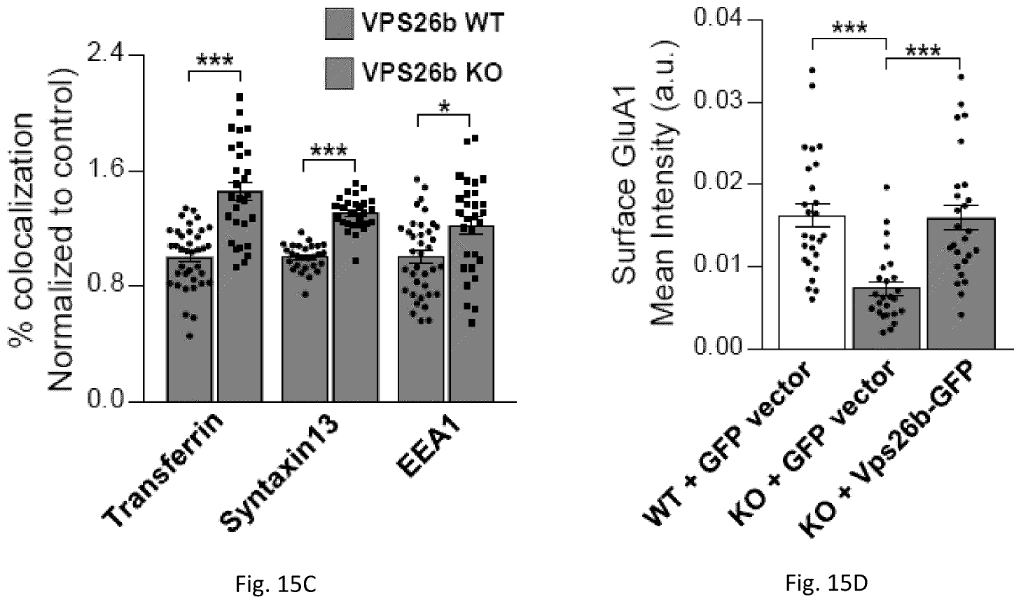

To begin testing this hypothesis, cell surface biotinylation experiments were performed in cells deficient for each VPS26 paralog. Consistent with the hypothesis, only VPS26b depletion, but not VPS26a deficiency, was found to result in a reduction of surface GluA1 levels (FIG. 15A). When cell surface level of GluA1 was normalized to the total amount of GluA1 in the cell lysate, it was found that surface GluA1 decreased by 35% in VPS26b-depleted neurons, while the GluA1 surface levels did not change in cells deficient for VPS26a (FIGS. 15A and 15B).

To further validate the hypothesis, using confocal microscopy it was found that VPS26b depletion causes a back log of GluA1 trafficking, with the greatest accumulation in recycling endosomes, and to a lesser degree in early endosomes (FIG. 15C). These results showed that in absence of VPS26b, GluA1 receptors are trapped in endosomes on their way to the cell surface.

Finally, to strengthen this mechanistic link, rescue experiments were performed using lentivirus to replete VPS26b in VPS26b KO neuronal cultures, finding that this repletion fully restored GluA1 localization at the cell surface (FIG. 15D). Collectively, these results showed that, compared to VPS26a, VPS26b is dedicated more to receptor recycling (Kennedy and Ehlers, 2011).

### Example 16—VPS26b in the AD-Targeted Trans-Entorhinal Cortex

While the TEC has been found to be differentially vulnerable to AD, this subregion and the entorhinal cortex as a whole, extends for over 4 centimeters, neighboring the hippocampus in its posterior aspect and the amygdala more anteriorly. In anticipation of a molecular investigation, the epicenter of the AD-targeted TEC needed to be pinpointed.

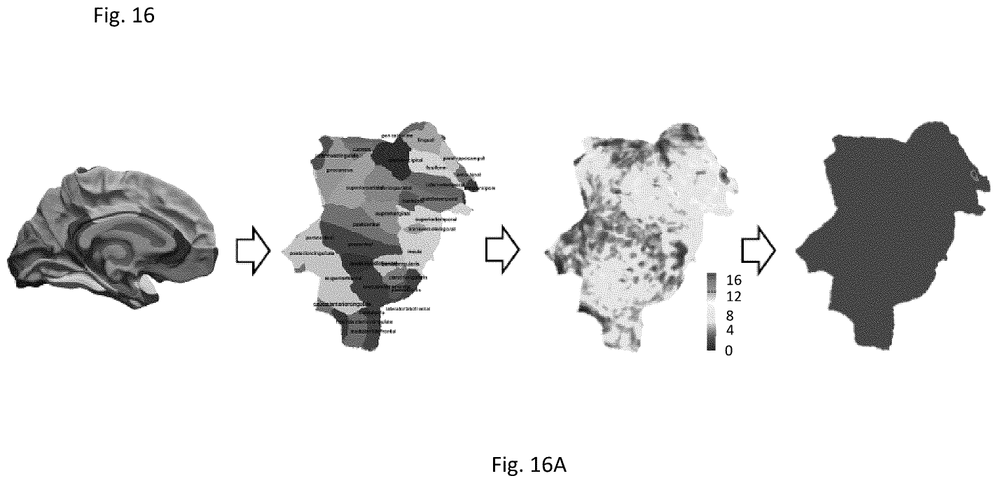

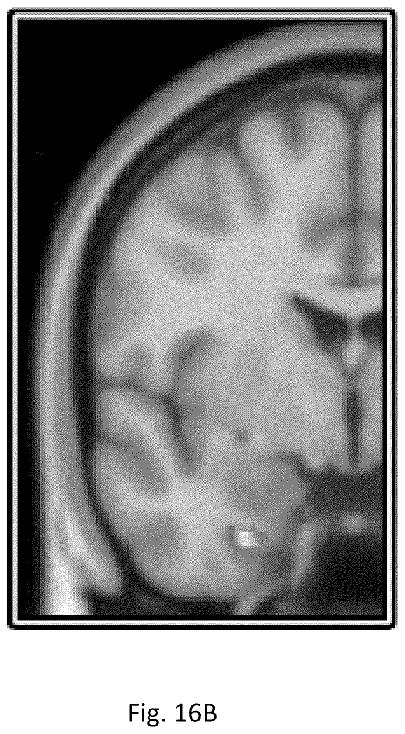

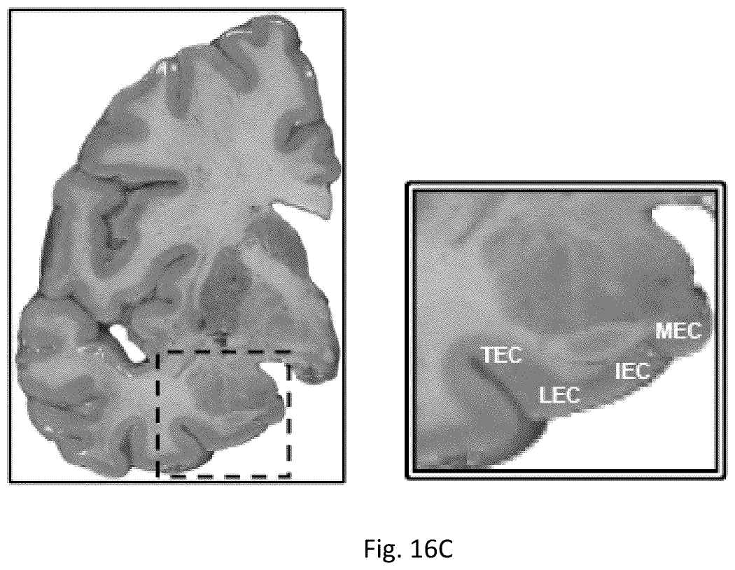

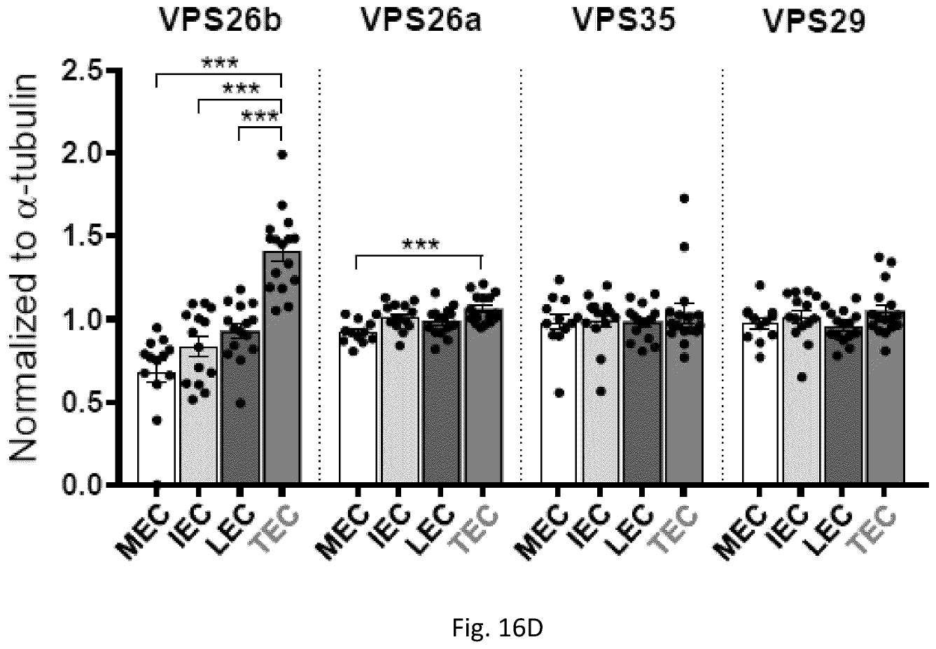

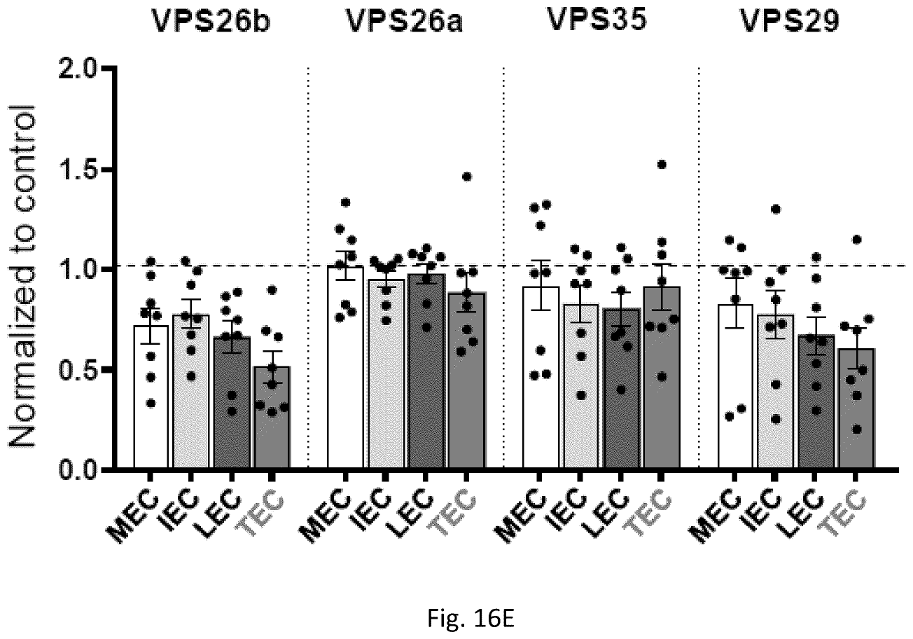

To do so, a large-scale MRI analysis was performed on data acquired from 188 AD cases and 169 healthy controls, and cortical thickness maps generated (FIG. 16A, left two panels). Comparing maps of AD vs. controls confirms that, across the brain, the vicinity of the TEC is the cortical area most reliably affected in AD (FIG. 16A, right two panels). By overlaying the statistical maps onto anatomical images, the epicenter was found to precisely localize to the anterior TEC, where it abuts the amygdala (FIG. 16B).

Guided by this anatomical precision, postmortem samples, of healthy subjects were identified, which extended into the anterior medial temporal lobe to include the AD-targeted TEC. From these samples the TEC and other subregions of the entorhinal cortex—the lateral entorhinal cortex (LEC), the intermediate entorhinal cortex (IEC) and the medial entorhinal cortex (MEC) were microdissected (FIG. 16C). Western blot analysis revealed that among the 4 retromer core proteins, only VPS26b expression was enriched in the TEC (FIG. 16D). In AD brains, retromer proteins showed the greatest reductions in the TEC, with the VPS26b showing the most reliable effect (FIG. 16E).

### Example 17— AAV9 VPS26b Rescued VPS26b in the VPS26b Knockout Mouse Model

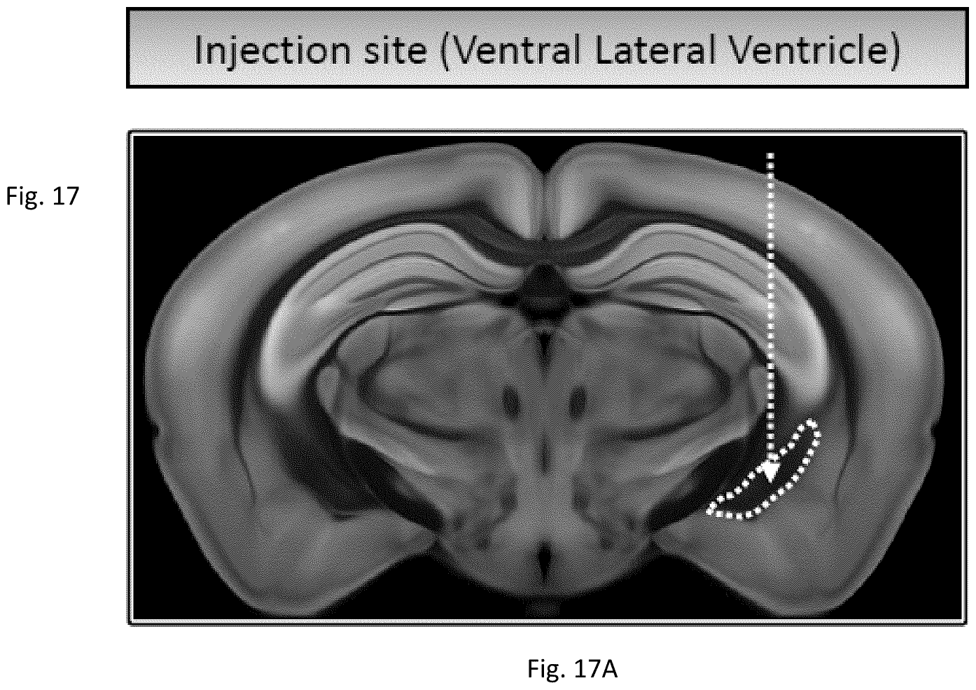

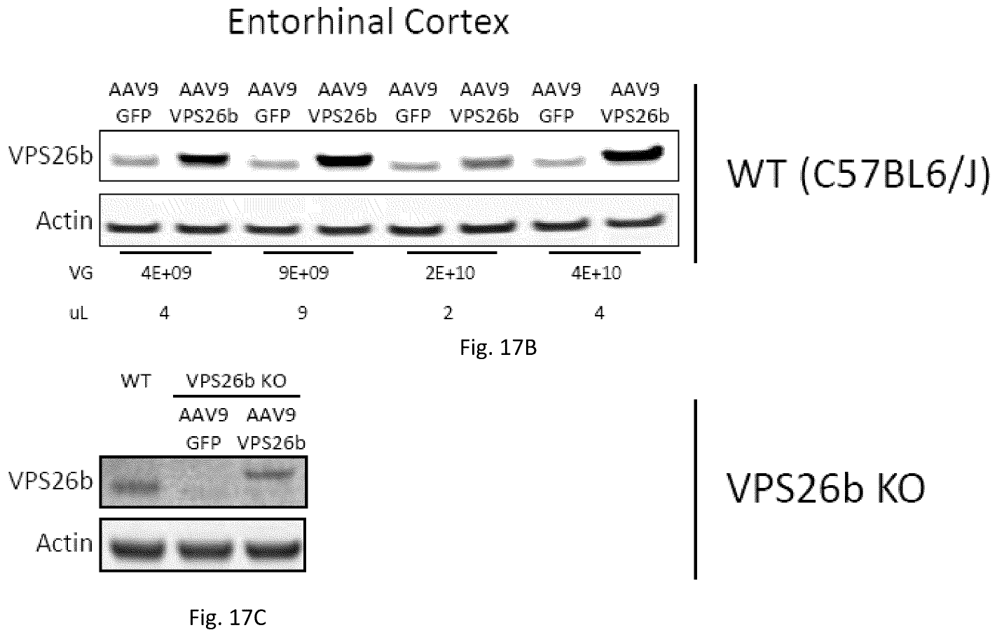

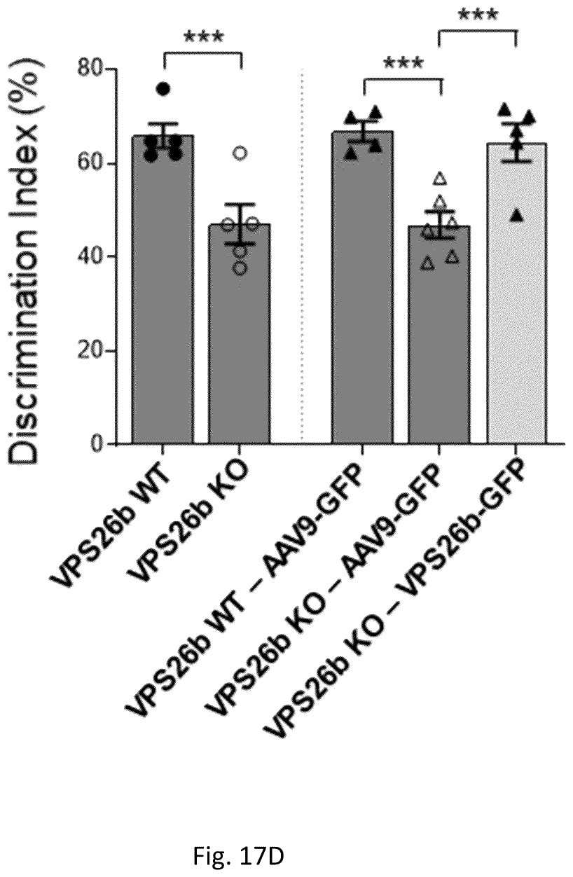

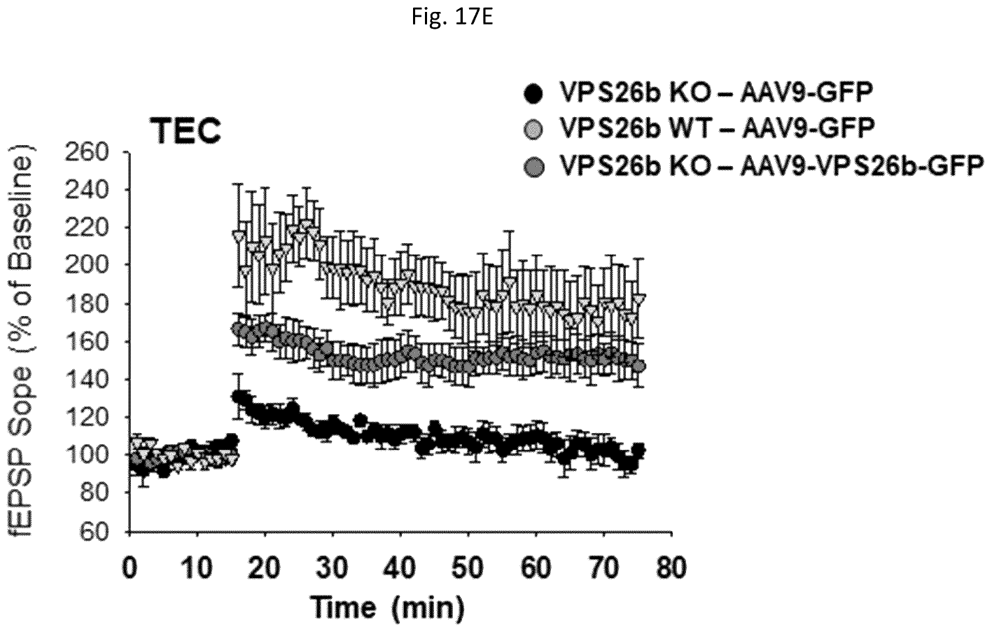

AAV9-VPS26b rescue experiments were conducted in mice in accordance with IACUC guidelines. Briefly the AAV9-CAG-VPS26b-2A-eGFP-WPRE vector was designed and finalized at Columbia University and then manufactured by Vector Biolabs. AAV9-CAG-eGFP-WPRE from Vector Biolabs was used as control. Viruses were injected in the brain as described previously (Qureshi et al., 2019). The injection coordinates were calculated to target the ventral recess of lateral ventricle using mouse brain atlas (FIG. 17A). Dose titration was performed in WT animals (FIG. 17B). Mice were injected at about 3.0 months and the brains harvested at about 4.0 months. Western blotting was performed as described in Example 9.

The optimal dose (4E+09 VG in 4 μl volume) was injected bilaterally in the experimental group which are VPS26b knock out mice as described in Example 9 and used for the studies in Examples 10-17. Animals were monitored post-surgery for side effects of the procedure. Mice were injected at about 3.7 months and the brains harvested at about 7.7 months. Western blotting was performed as described in Example 9.

As shown in FIG. 17C, the administration of the AAV9 VPS26b vector to the VPS26b KO restored VPS26b knock out levels.

As shown in FIG. 17D, mice aged for 3 months post-surgery and were behaviorally tested using OCR as described in Example 9. AAV9-GFP was injected in control animals Replicating the previous findings, compared to the VPS26b WT animals injected with AAV9-GFP, the VPS26b KO mice injected with AAV9-GFP had OCR defect, but when injected with AAV9-VPS26b, the behavioral defect was completely normalized (FIG. 17D).

Lastly, to confirm the mechanistic underpinnings of this behavioral rescue, the electrophysiological effect of VPS26b repletion was tested. The LTP defect in the TEC was normalized in VPS26b KO mice, when injecting AAV9-VPS26b compared to mice injected with AAV9-GFP (FIG. 17E).

Together, these results suggested that the TEC's function is differentially dependent on VPS26b.

### Example 18— APLP1 is a CSF Marker of Endosomal Pathology in Parkinson's Disease

CSF was extracted from cisterna magna of VPS35 D620N Parkinson's disease variant heterozygous mice and WT littermate controls in accordance with IACUC guidelines. Briefly, anesthetized mice were placed in a prone position and the skin covering the back of the neck, shaved. A cotton swab containing 70% ethanol was used to remove any hair from the exposed skin. Then, a 27-gauge sterile needle (SV*27EL, Terumo Medical Products) attached to a 1-ml syringe (329650, BD Biosciences) was inserted into the cisterna magna allowing flow of CSF into the butterfly needle. After 10-15 seconds, the needle was removed and the CSF aspirated into microcentrifuge tubes (1605-0000, USA Scientific), which was immediately placed on dry ice and subsequently stored at −80C. Roughly 5-10 uL of fluid was collected per mouse. CSF visibly contaminated with blood was discarded. All remaining samples underwent more stringent assessment for blood contamination via hemoglobin ELISA using 0.5 uL in a 1:200 dilution. Samples below 0.01% blood contamination were retained for analyses and 6 μl was resolved on Western blot and probed for APLP1 and Albumin antibodies as described in Example 1. Total protein levels were assessed using Ponceau-S stain.

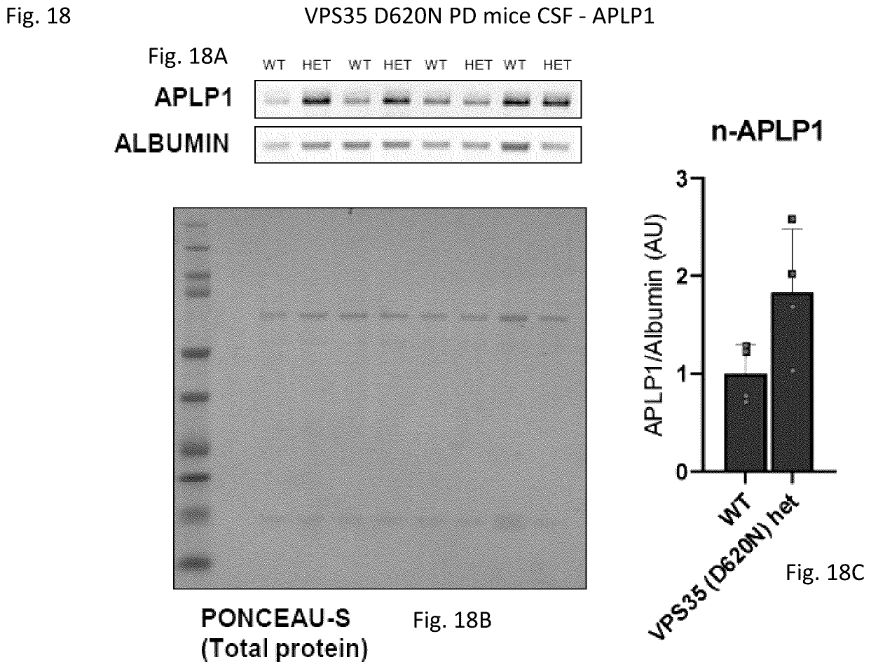

As shown in FIG. 18, the CSF of VPS35 D620N Parkinson's disease variant heterozygous mice contained much greater amounts of n-APLP1 protein.

### REFERENCES

Alamed et al., 2006. Two-day radial-arm water maze learning and memory task; robust resolution of amyloid related memory deficits in transgenic mice. Nat Protoc, 1, 1671-9.
Avants et al., 2011. A reproducible evaluation of ANTs similarity metric performance in brain image registration. Neuroimage, 54, 2033-44.
Barker and Warburton, 2011. When is the hippocampus involved in recognition memory? J Neurosci, 31, 10721-31.
Barthélemy et al., 2016a. Tau protein quantification in human cerebrospinal fluid by targeted mass spectrometry at high sequence coverage provides insights into its primary structure heterogeneity. J. Proteome Res 15, 667-676.
Barthélemy et al., 2016b. Differential mass spectrometry profiles of tau protein in the cerebrospinal fluid of patients with Alzheimer's disease, progressive supranuclear palsy, and dementia with Lewy bodies. J. Alzheimer Dis 51, 1033-1043.
Bayer et al., 1999. It all sticks together—the APP-related family of proteins and Alzheimer's disease. Mol. Psychiatry 4, 524-528.
Bolte and Cordelieres, 2006. A guided tour into subcellular colocalization analysis in light microscopy. J Microsc, 224, 213-32.
Braak and Braak, 1991. Neuropathological stageing of Alzheimer-related changes. Acta Neuropathol (Berl), 82, 239-59.
Bugarcic et al., 2011. Vps26A and Vps26B subunits define distinct retromer complexes. Traffic, 12, 1759-1046.
Cepeda-Prado et al., 2012. R6/2 Huntington's disease mice develop early and progressive abnormal brain metabolism and seizures. J Neurosci, 32, 6456-67.
Chai et al., 2012. Constitutive secretion of tau protein by an unconventional mechanism. Neurobiol. Dis 48, 356-366.
Chen et al., 2019. Learnings about the complexity of extracellular tau aid development of a blood-based screen for Alzheimer's disease. Alzheimers Dement. 15, 487-496.
Chou et al., 2011. Robust automatic rodent brain extraction using 3-D pulse-coupled neural networks (PCNN). IEEE Trans Image Process, 20, 2554-64.
Cicognola et al., 2019. Novel tau fragments in cerebrospinal fluid: Relation to tangle pathology and cognitive decline in Alzheimer's disease. Acta Neuropathol. 137, 279-296.
Dislich et al., 2015. Label-free quantitative proteomics of mouse cerebrospinal fluid detects β-site APP cleaving enzyme (BACE1) protease substrates in vivo. Mol. Cell. Proteomics 14, 2550-2563.
Fischl and Dale, 2000. Measuring the thickness of the human cerebral cortex from 30 1068 magnetic resonance images. Proc Natl Acad Sci USA, 97, 11050-5.
Fischl et al., 1999. Cortical surface-based analysis. II: Inflation, flattening, and a surface-based coordinate system. Neuroimage, 9, 195-207.
Gallon et al., 2014. A unique PDZ domain and arrestin-like fold interaction reveals mechanistic details of endocytic recycling by SNX27-retromer. Proc Natl Acad Sci USA, 111, E3604-13.
Jack et al., 2008. The Alzheimer's Disease Neuroimaging Initiative (ADNI): MRI methods. J Magn Reson Imaging, 27, 685-91.
Karch et al., 2012. Extracellular Tau levels are influenced by variability in Tau that is associated with tauopathies. J. Biol. Chem 287, 42751-42762.
Kennedy and Ehlers, 2011 Mechanisms and function of dendritic exocytosis. Neuron, 69, 856-75.
Khan et al. 2014. Molecular drivers and cortical spread of lateral entorhinal cortex dysfunction in preclinical Alzheimer's disease. Nat Neurosci, 17, 304-11.
Kim et al., 2010. Implication of mouse Vps26b-Vps29-Vps35 retromer complex in sortilin trafficking. Biochem Biophys Res Commun, 403, 167-71.
Kramer et al., 2014. Causal analysis approaches in Ingenuity Pathway Analysis. Bioinformatics 30, 523-530.
Li et al., 2019, Atypical parkinsonism-associated retromer mutant alters endosomal sorting of specific cargo proteins. J Cell Biol, 214, 389-99.
Mai et al., 2015. Atlas of the Human Brain, Academic Press.
McMillan et al., 2016. Atypical parkinsonism-associated retromer mutant alters endosomal sorting of specific cargo proteins. J Cell Biol, 214, 389-99.
Mecozzi et al., 2014. Pharmacological chaperones stabilize retromer to limit APP processing. Nat Chem Biol, 10, 443-9.
Meredith et al., 2013. Characterization of novel CSF Tau and ptau biomarkers for Alzheimer's disease. PLOS ONE 8, e76523.
Mirra et al., 1991. The Consortium to Establish a Registry for Alzheimer's Disease (CERAD). Part II. Standardization of the neuropathologic assessment of Alzheimer's disease. Neurology, 41, 479-86.
Mohammed et al., 2014. Starvation and inhibition of lysosomal function increased tau secretion by primary cortical neurons. Sci. Rep 4, 5715.
Mohammed et al., 2008. Retromer deficiency observed in Alzheimer's disease causes hippocampal dysfunction, neurodegeneration, and Abeta accumulation. Proc Natl Acad Sci USA, 105, 7327-32.
Muzio et al., 2020. Retromer stabilization results in neuroprotection in a model of Amyotrophic Lateral Sclerosis. Nature Communication 11, 3848.
Qureshi et al., 2019 Retromer repletion with AAV9-VPS35 restores endosomal function in the mouse hippocampus. bioRxiv 2019:618496.
Rissin et al., 2010. Single-molecule enzyme-linked immunosorbent assay detects serum 25 proteins at subfemtomolar concentrations. Nat. Biotechnol 28, 595-599.
Sabuncu et al., 2009. Asymmetric Image-Template Registration. Medical image computing and computer assisted intervention: MICCAI . . . International Conference on Medical Image Computing and Computer-Assisted Intervention, 12, 565-573.
Seaman, 2012. The retromer complex—endosomal protein recycling and beyond. J Cell Sci, 125, 4693-702.
Small and Petsko, 2015. Retromer in Alzheimer disease, Parkinson disease and other neurological disorders. Nat Rev Neurosci, 16, 126-32.
Small et al., 2011. A pathophysiological framework of hippocampal dysfunction in ageing and disease. Nat Rev Neurosci, 12, 585-601.
Small et al., 2017. Endosomal Traffic Jams Represent a Pathogenic Hub and Therapeutic Target in Alzheimer's Disease. Trends Neurosci, 40, 592-602.
SStti et al., 2018. Tau kinetics in neurons and the human central nervous system. Neuron 98, 861-864.
Temkin et al., 2017. The Retromer Supports AMPA Receptor Trafficking During LTP. Neuron, 94, 74-82 e5.
Triana-Baltzer et al. 2020. Development and validation of a high sensitivity assay for measuring p217+tau in cerebrospinal fluid. J. Alzheimers Dis 77, 1417-1430.
Truschl et al., 2009. ESCRT-I function is required for Tyrpl transport from early endosomes to the melanosome limiting membrane. Traffic, 10, 1318-36.
Vagnozzi et al., 2019 VPS35 regulates tau phosphorylation and neuropathology in tauopathy. Mol Psychiatry.
Vardarajan et al., 2012. Identification of Alzheimer disease-associated variants in genes that regulate retromer function. Neurobiol. Aging 33, 2231.e15-2231.e30.
Vassar et al., 1999. β-secretase cleavage of Alzheimer's amyloid precursor protein by the transmembrane aspartic protease BACE. Science 286, 735-741.
Vonsattel et al., 1995. An improved approach to prepare human brains for research. J Neuropathol Exp Neurol, 54, 42-56.
Wang et al., 2012. VPS35 regulates developing mouse hippocampal neuronal morphogenesis by promoting retrograde trafficking of BACE1. Biol. Open 1, 1248-1257.
Wilson et al., 2013. Lateral entorhinal cortex is critical for novel object-context recognition. Hippocampus, 23, 352-66.
Young et al., 2018, Stabilizing the Retromer Complex in a Human Stem Cell Model of Alzheimer's Disease Reduces TAU Phosphorylation Independently of Amyloid Precursor Protein. Stem Cell Reports, 10, 1046-1058.
Zhang et al., 2014. An RNA-sequencing transcriptome and splicing database of glia, neurons, and vascular cells of the cerebral cortex. J Neurosci, 34, 11929-47.

1. A method of treating, preventing and/or curing Alzheimer's disease in a subject in need thereof, comprising:
a. detecting or measuring the amount or level of one or more biomarkers in a sample from the subject, wherein the one or more biomarkers identify retromer dysfunction and/or endosomal trafficking dysfunction involvement and/or implication in Alzheimer's disease;
b. comparing the amount or level of the one or more biomarkers in the sample to a reference amount or level of one or more of the biomarkers, wherein if the amount or level of the one or more biomarkers in the sample is different than the reference amount or level of one or more biomarkers from a healthy control, or if the amount or level of the one or more biomarkers in the sample is the same as the reference amount or level of one or more biomarkers from a subject suffering from Alzheimer's disease where retromer dysfunction and/or endosomal trafficking dysfunction is involved and/or implicated, the subject is suffering from Alzheimer's disease where retromer dysfunction is involved and/or implicated; and
c. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

2. (canceled)

3. A method of treating, preventing and/or curing Alzheimer's disease in a subject in need thereof, comprising:
a. purifying and/or isolating protein from a sample from the subject;
b. detecting or measuring the amount or level of the N-terminal fragment of CHL1;
c. comparing the amount or level of the N-terminal fragment of CHLI from the sample to a reference amount or level of the N-terminal fragment of CHLI;
d. detecting or measuring the amount or level of the N-terminal fragment of APLPI;
e. comparing the amount or level of the N-terminal fragment of APLPI from the sample to a reference amount or level of the N-terminal fragment of APLPI; and
f. detecting that the subject has retromer dysfunction and/or endosomal trafficking dysfunction when the amount or level of the N-terminal fragment of CHLI and the amount or level of the N-terminal fragment of APLPI are increased compared to the reference amount or level, wherein the reference amounts or levels are from a healthy control; and
g. treating the subject with a retromer therapeutic selected from the group consisting of a composition comprising a nucleic acid or transgene encoding VPS35, a composition comprising a nucleic acid or transgene encoding VPS26b, a composition comprising a viral vector comprising a nucleic acid or transgene encoding VPS35, a composition comprising a viral vector comprising a nucleic acid or transgene encoding VPS26b, a pharmacological chaperone which increases VPS35 and/or VPS26b, and combinations thereof.

4. The method of claim 3, further comprising detecting or measuring the amount or level of tau; and comparing the amount or level of tau from the sample to a reference amount or level of tau.

5. The method of claim 3, wherein the amount or level of APLPI and CHLI are measured using a SIMOA™ assay.

6. A method of treating, preventing and/or curing Parkinson's disease in a subject in need thereof, comprising:
a. detecting or measuring the amount or level of one or more biomarkers in a sample from the subject, wherein the one or more biomarkers identify retromer dysfunction and/or endosomal trafficking dysfunction involvement and/or implication in Parkinson's disease;
b. comparing the amount or level of the one or more biomarkers in the sample to a reference amount or level of one or more of the biomarkers, wherein if the amount or level of the one or more biomarkers in the sample is different than the reference amount or level of one or more biomarkers from a healthy control, or if the amount or level of the one or more biomarkers in the sample is the same as the reference amount or level of one or more biomarkers from a subject suffering from Parkinson's disease where retromer dysfunction and/or endosomal trafficking dysfunction is involved and/or implicated, the subject is suffering from Parkinson's disease where retromer dysfunction and/or endosomal trafficking dysfunction is involved and/or implicated; and
c. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

7. (canceled)

8. A method of treating, preventing and/or curing Parkinson's disease in a subject in need thereof, comprising:
a. purifying and/or isolating protein from a sample from the subject;
b. detecting or measuring the amount or level of the N-terminal fragment of APLPI;
c. comparing the amount or level of the N-terminal fragment of APLPI from the sample to a reference amount or level of the N-terminal fragment of APLPI;
d. detecting that the subject has Parkinson's disease where retromer dysfunction and/or endosomal trafficking dysfunction is involved and/or implicated when the amount or level of the N-terminal fragment of APLPI is increased compared to the reference amount or level and wherein the reference amount or level is from a healthy control; and
e. treating the subject with a retromer therapeutic selected from the group consisting of a composition comprising a nucleic acid or transgene encoding VPS35, a composition comprising a nucleic acid or transgene encoding VPS26a, a composition comprising a viral vector comprising a nucleic acid or transgene encoding VPS35, a composition comprising a viral vector comprising a nucleic acid or transgene encoding VPS26a, a pharmacological chaperone which increases VPS35 and/or VPS26a, and combinations thereof.

9. The method of claim 3, wherein the retromer core protein VPS35 encoded by the transgene or nucleic acid has the amino acid sequence of VPS35 (SEQ ID NO: 1).

10. The method of claim 3, wherein the retromer core protein VPS35 encoded by the transgene or nucleic acid has an amino acid sequence that is at least 85% identical to the amino acid sequence of VPS35 (SEQ ID NO:1).

11. The method of claim 3, wherein the transgene or nucleic acid has a nucleic acid sequence that is at least 70% identical to the nucleic acid sequence encoding VPS35 (SEQ ID NO: 1).

12. (canceled)

13. The method of claim 8, wherein the retromer core protein VPS26a encoded by the transgene or nucleic acid has an amino acid sequence that is at least 85% identical to the amino acid sequence of VPS26a (SEQ ID NO: 4).

14. The method of claim 8, wherein the transgene or nucleic acid has a nucleic acid sequence that is at least 70% identical to the nucleic acid sequence encoding VPS26a (SEQ ID NO: 4).

15. The method of claim 3, wherein the retromer core protein VPS26b encoded by the transgene or nucleic acid has an amino acid sequence that is at least 85% identical to the amino acid sequence of VPS26b (SEQ ID NO: 7).

16. The method of claim 3, wherein the retromer core protein VPS26b encoded by the transgene or nucleic acid has the amino acid sequence of VPS26b (SEQ ID NO: 7).

17. The method of claim 3, wherein the transgene or nucleic acid has a nucleic acid sequence that is at least 70% identical to the nucleic acid sequence encoding VPS26b (SEQ ID NO: 7).

18. The method of claim 3, wherein the vector is selected from the group consisting of an adeno-associated virus (AAV), adenovirus, lentivirus, retrovirus, poxvirus, baculovirus, herpes simplex virus, vaccinia virus, and a synthetic virus.

19. (canceled)

20. The method of claim 18, wherein the AAV is an AAV1, AAV2, AAV4, AAV5, AAV6, AAV7, AAV8, AAV9, AAV10, or AAVrh10.

21. The method of claim 3, wherein the transgene or nucleic acid is operably linked to a promoter or enhancer that induces expression of the transgene in a neural cell.

22. (canceled)

23. The method of claim 3, wherein the pharmacological chaperone is selected from the group consisting of small molecules, chemicals, pharmaceuticals, biologics, antibodies, nucleic acids, peptides, and proteins.

24. The method of claim 3, wherein the pharmacological chaperone binds at the interface between VPS35 and VPS29.

25. The method of claim 3, wherein the pharmacological chaperone is selected from the group consisting of R55 and R33.


SEQUENCE LISTING (11 sequences)

SEQ ID NO: 1 - protein, 796 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 2 - DNA, 2391 bp, Mus musculus
atgcctacaacacagcagtcaccccaggatgagcaggaaaaacttttggatgaagccatccaggctgtgaaggttcagtcattccagatgaaaagatgcctggacaaaaacaagctgatggatgctctgaagcatgcctccaatatgcttggagagctccggacctctatgctgtcaccaaagagttactatgaactttatatggctatttctgatgaactgcactacttggaggtctacctgactgatgaatttgctaaaggaagaaaggtggcagatctctatgaacttgtacagtacgcgggaaacattattccaaggctttatctcttgatcacagttggagttgtatatgtcaagtcatttcctcaatccaggaaagatattttgaaagatttggtagaaatgtgccgtggtgtgcagcatccgctaaggggtttgtttcttcgaaattaccttcttcagtgtactaggaacattttacctgatgaaggagagccaacagatgaagaaacaactggtgatatcagtgattccatggattttgtactactcaactttgcagaaatgaataagctctgggtgcggatgcagcatcaaggacatagtcgagatagagaaaaaagagaacgagagagacaagaactgagaattttagtgggaacaaatttggtgcgccttagtcagttggaaggtgtaaatgtggaacgttacaaacagattgttctaacaggcattctggagcaggttgtgaattgtagggatgctctggctcaggagtatctcatggagtgcatcattcaggtttttcctgatgagtttcatcttcagactttgaatccttttcttagagcttgtgctgaattacaccaaaatgtaaatgtgaaaaacataatcattgctttaattgacagattagctttatttgctcatcgtgaagatggacccggaattccagctgagattaaactttttgacatattttcacaacaggtggctacagtcatacagtccagacaagacatgccatcagaggatgttgtatctttacaagtctctctcattaatcttgctatgaaatgttaccctgatcgtgtggactacgtcgataaagttctagaaacaacagtggagatattcaataagcttaaccttgaacatattgctaccagtagtgcagtttcaaaggagcttaccagacttttgaagatacctgttgatacttacaacaatatcttaacagtattaaagttaaagcatttccacccactttttgagtactttgactacgagtccagaaagagcatgagctgttatgtgcttagtaatgttctggattataacacagaaatcgtctctcaggaccaggtagattccataatgaatttggtgtccacattgattcaggatcagccagaccaacctgtagaagaccctgatccagaagacttcgctgatgaacagagccttgttggccgatttattcatctgctacgttctgatgatcctgaccagcagtatttgattttgaatacggcacgaaaacattttggggctggtggaaatcagcggattcgcttcacactgccacctttggtatttgcagcttaccagttggcttttcgatacaaagagaattcccaaatggatgacaagtgggaaaagaaatgccagaagatattttcatttgctcaccagactatcagtgctttgattaaagctgagctggctgaattaccactgagactttttcttcaaggagcattagctgctggagaaattggctttgaaaatcatgaaacagtagcatatgaatttatgtcccaggcattttctctatatgaagatgaaatcagtgattctaaagcacagctggctgctatcaccttgatcatcggtacttttgagaggatgaaatgcttcagtgaagagaatcatgaacccttgagaactcagtgtgcacttgctgcatcaaaacttctgaaaaaaccagatcaaggccgagccgtgagcacatgtgcgcatctcttttggtctggccgaaacacagacaaaaatggggaagagcttcatggaggtaaaagggtcatggagtgcctaaagaaggcactaaaaatagcaaatcagtgcatggacccctctctacaagttcagctctttatagagattctgaacaggtacatctatttctatgaaaaagaaaatgatgcggtaaccattcaagtcttgaaccaacttattcaaaagattcgagaagatctcccaaatcttgagtccagtgaagaaacagaacaaataaacaagcattttcacaacacattggagcacttgcgctcaagacgggaatcaccagagtctgaggggccaatctatgaaggtctcatcctttaa

SEQ ID NO: 3 - DNA, 2391 bp, Artificial Sequence
  synthetic nucleotide
atgcccacaacacagcagagcccacaggatgaacaggaaaagctgctggatgaggccatccaggctgtgaaagtgcagtccttccagatgaaaagatgtctggataagaataagctgatggacgccctgaagcatgccagcaacatgctgggcgagctgaggaccagcatgctgtctcccaagtcctactacgagctgtacatggccatttctgacgagctgcactacctggaggtgtacctcacagatgagttcgccaaggggagaaaggtggccgatctgtatgagctggtgcagtatgctggcaacatcatccccaggctgtacctgctgatcaccgtgggcgtggtgtatgtgaagagcttcccccagtctagaaaggatatcctgaaggacctggtggaaatgtgcaggggagtgcagcatcctctgagaggcctgtttctgagaaactacctgctgcagtgtaccaggaatatcctgcccgacgaaggcgagcctactgatgaagaaacaaccggcgacatcagcgacagcatggattttgtgctgctgaacttcgccgagatgaacaaactgtgggtgagaatgcagcaccagggacacagcagggacagagagaagagagagagggagagacaggagctgagaatcctggtgggcaccaacctggtgaggctgtcccagctggagggcgtgaatgtggagagatataagcagattgtgctgacaggaatcctggagcaggtggtgaactgcagagatgccctggcccaggagtatctgatggaatgcatcattcaggtgttcccagatgagttccacctgcagacactgaaccctttcctgagggcctgcgccgagctgcatcagaacgtgaacgtcaagaacatcatcattgctctgattgacaggctggccctgtttgcccatagagaggatggacctggcatcccagccgagatcaagctgtttgacattttcagccagcaggtggccaccgtgatccagtctaggcaggacatgccttccgaagatgtggtgagcctgcaggtgtccctgatcaatctggccatgaagtgttaccctgatagagtggattacgtggacaaggtgctggagaccacagtggagatcttcaacaagctgaacctggagcacatcgccacttcttctgctgtgtctaaagaactgacaagactgctgaagatccctgtggacacctacaacaacattctgaccgtgctgaagctgaaacacttccatccactgttcgagtactttgattatgagagcaggaagtccatgagctgctacgtgctgagcaatgtgctggactacaatacagaaatcgtgtcccaggatcaggtggacagcatcatgaatctggtgagcacactgatccaggaccagcctgatcagccagtggaggaccccgaccctgaggacttcgctgacgagcagagcctggtgggcaggttcatccacctgctgaggtctgatgatccagaccagcagtacctgatcctgaacaccgccaggaagcactttggagccggaggcaaccagaggatcagattcaccctgcctcccctggtgtttgccgcctaccagctggctttcagatacaaggaaaattcccagatggatgacaagtgggagaaaaaatgccagaagatcttttcttttgctcaccagaccatctctgccctgatcaaagctgagctggctgaactgccactgagactgttcctccagggggccctggctgccggggagatcggctttgagaaccatgagactgtggcctatgaattcatgtctcaggccttctctctgtacgaagatgagatcagcgacagcaaggctcagctggctgctatcacactgatcattggaacatttgaaaggatgaagtgcttctccgaggaaaaccacgagccactgagaacacagtgtgcactggccgcctccaagctgctgaagaagcctgaccagggcagagccgtgtctacctgtgcccacctgttctggagcggaagaaacacagacaaaaatggagaggagctgcatggcggcaagagggtgatggagtgcctgaagaaagccctgaagattgccaaccagtgcatggacccaagcctgcaggtgcagctgttcatcgagatcctgaacaggtacatctacttctacgagaaggagaatgacgccgtgaccattcaggtgctgaatcagctgatccagaaaatcagggaagacctgcccaacctggagagctctgaagagacagagcagatcaacaagcattttcacaacactctggaacatctgaggtccaggagggagtccccagagtctgagggacccatctatgaggggctgatcctgtga

SEQ ID NO: 4 - protein, 327 aa, Homo sapiens
MSFLGGFFGPICEIDIVLNDGETRKMAEMKTEDGKVEKHYLFYDGESVSGKVNLAFKQPGKRLEHQGIRIEFVGQIELFNDKSNTHEFVNLVKELALPGELTQSRSYDFEFMQVEKPYESYIGANVRLRYFLKVTIVRRLTDLVKEYDLIVHQLATYPDVNNSIKMEVGIEDCLHIEFEYNKSKYHLKDVIVGKIYFLLVRIKIQHMELQLIKKEITGIGPSTTTETETIAKYEIMDGAPVKGESIPIRLFLAGYDPTPTMRDVNKKFSVRYFLNLVLVDEEDRRYFKQQEIILWRKAPEKLRKQRTNFHQRFESPESQASAEQPEM

SEQ ID NO: 5 - DNA, 984 bp, Mus musculus
atgagttttcttggaggcttttttggtcccatttgtgagattgatgttgcccttaatgatggggaaaccaggaaaatggcagaaatgaaaactgaggatggcaaagtagaaaaacactatctcttctatgatggcgagtctgtctcaggaaaggtaaacctagcctttaagcagcctggaaagaggctagagcatcaaggaattagaattgaatttgtaggtcaaattgagcttttcaatgacaagagtaatactcatgaatttgtaaacctagtgaaggaactagccttgcctggagaactgactcagagcagaagctatgactttgaatttatgcaagttgaaaagccatatgagtcatacatcggtgccaatgtccgcctgaggtattttcttaaggtgacaattgtgagaagattgacagacttagtgaaagagtacgatcttattgttcatcagctagccacctatcctgatgtcaacaactctattaaaatggaagtgggcattgaagactgtctgcacatagagtttgaatataataagtccaagtatcatttaaaggatgtaattgttggaaaaatttacttcttattagtaagaataaaaatacaacacatggaattacagctgatcaagaaagagatcacaggaattggacccagcaccacaacagagacagaaacaatcgctaagtatgaaataatggatggggcgccagtaaaaggagaatctattccgataagattgttcttagcagggtatgacccaacccccacgatgagagatgtgaacaagaagttttcagtaaggtactttctaaacctcgtgcttgttgatgaggaggaccgaaggtacttcaagcagcaggagatcatcctgtggagaaaagcacccgagaaactgagaaaacagaggacgaactttcaccagcggtttgaatctccagactcgcaggcctctgcggagcagcctgagatgtaa

SEQ ID NO: 6 - DNA, 984 bp, Artificial Sequence
  synthetic nucleotide
atgagcttcctgggcggcttcttcggccccatctgtgagatcgacgtggccctgaacgacggcgagaccagaaagatggccgagatgaagacagaggatggcaaggtggagaagcactacctgttctacgacggagagtctgtgtccggcaaggtgaacctggccttcaagcagcctgggaagaggctggagcaccagggcatcagaatcgagttcgtgggccagatcgagctgttcaacgacaagagcaacacccacgagtttgtgaacctggtgaaggagctggctctgcctggcgagctgacccagagcagaagctacgacttcgagttcatgcaggtggagaagccttacgagagctacatcggcgccaacgtgagactgagatacttcctgaaggtgaccatcgtgaggagactgaccgacctggtgaaggagtatgacctgatcgtgcaccagctggccacctaccctgacgtgaacaacagcatcaagatggaggtgggcatcgaggactgcctgcacatcgagttcgagtacaacaagtccaagtaccacctgaaggacgtgatcgtgggcaagatctacttcctgctggtgaggatcaagatccagcacatggagctgcagctgatcaagaaggagatcaccggcatcggcccttccacaaccaccgagacagagacaatcgccaagtacgagatcatggacggcgcccctgtgaagggcgagagcatccctatcaggctgttcctggccggctacgaccctacccctaccatgagagacgtgaacaagaagttcagcgtgaggtacttcctgaacctggtgctggtggacgaggaggacagaagatacttcaagcagcaggagatcatcctgtggaggaaggcccctgagaagctgaggaagcagaggaccaacttccaccagagattcgagtcccctgacagccaggccagcgccgagcagccagagatgtga

SEQ ID NO: 7 - protein, 336 aa, Homo sapiens
MSFFGFGQSVEVEILLNDAESRKRAEHKTEDGKKEKYFLFYDGETVSGKVSLALKNPNKRLEHQGIKIEFIGQIELYYDRGNHHEFVSLVKDLARPGEITQSQAFDFEFTHVEKPYESYTGQNVKLRYFLRATISRRLNDVVKEMDIVVHTLSTYPELNSSIKMEVGIEDCLHIEFEYNKSKYHLKDVIVGKIYFLLVRIKIKHMEIDIIKRETTGTGPNVYHENDTIAKYEIMDGAPVRGESIPIRLFLAGYELTPTMRDINKKFSVRYYLNLVLIDEEERRYFKQQEVVLWRKGDIVRKSMSHQAAIASQRFEGTTSLGEVRTPSQLSDNNCRQ

SEQ ID NO: 8 - DNA, 1011 bp, Mus musculus
atgagcttcttcggcttcgggcagagcgtggaggtggaaatcttgctgaatgatgcggagagtaggaagcgagcggagcacaagactgaggacgggaagaaggagaaatatttcctcttctacgacggggaaaccgtctcggggaaagtgagcctatcactgaagaaccccaacaagcgactagagcaccagggcatcaagatcgagttcattgggcagatcgaactctactatgaccgtgggaaccaccatgagtttgtgtctttggtgaaggacctggctcggccaggagagatcacccaatcgcaggccttcgactttgagttcacccatgtggaaaagccgtatgaatcctacacaggacagaatgtgaagctccgctatttccttcgagccaccatcagccgccgcctcaatgatgttgttaaagagatggacattgtagttcacacacttagcacataccccgagctgaactcatccatcaagatggaagttggcattgaggattgcctgcacattgaatttgagtacaacaaatccaaataccacttgaaagatgtcattgtagggaagatatacttcctgctggtgagaatcaagatcaagcacatggagatagacatcatcaaacgagagacaacaggcacgggtcccaacgtgtaccacgagaacgacacaatagcgaagtatgagatcatggacggggcaccagtccgaggtgagtccatccccatcaggctcttcctggcagggtatgagctcacacccaccatgcgtgacataaataagaagttctctgtgcgctattacctcaacttggtgctgatagatgaggaggaacggcgctacttcaagcagcaggaagtggtgttgtggcggaagggtgacatcgtacggaagagcatgtcccaccaggcagccattgcctcacagcgcttcgagggcacaacctccctgggtgaggtgcggacccctggccaactgtctgacaacaacagcaggcagtag

SEQ ID NO: 9 - DNA, 1011 bp, Artificial Sequence
  synthetic nucleotide
atgagtttttttgggtttggacagtcagtggaggtggagatcctgctgaacgacgccgagagcaggaagagggccgagcacaagacagaggatggcaagaaggagaagtacttcctgttctacgacggagagaccgtgagcggcaaggtgtccctgagcctgaagaacccaaacaagagactggagcaccagggcatcaagatcgagttcatcggccagatcgagctgtactacgacaggggcaaccaccacgagttcgtgagcctggtgaaggacctggccagacctggcgagatcacccagagccaggccttcgacttcgagttcacccacgtggagaagccttacgagagctacacaggccagaacgtgaagctgaggtacttcctgagagccaccatcagcagaagactgaacgacgtggtgaaggagatggacatcgtggtgcacaccctgagcacctaccctgagctgaactcttccatcaagatggaggtgggcatcgaggactgcctgcacatcgagttcgagtacaacaagagcaagtaccacctgaaggacgtgatcgtgggcaagatctacttcctgctggtgagaatcaagatcaagcacatggagatcgacatcatcaagagagagaccacaggcacaggccctaacgtgtaccacgagaacgacaccatcgccaagtacgagatcatggacggcgcccctgtgagaggcgagagcatccctatcaggctgttcctggccggctacgagctgacccctaccatgagggacatcaacaagaagttcagcgtgagatactacctgaacctggtgctgatcgatgaggaggagagaagatacttcaagcagcaggaggtggtgctgtggagaaagggcgacatcgtgagaaagagcatgagccaccaggccgccatcgccagccagagattcgagggcaccaccagcctgggcgaggtgaggacccctggccagctgagcgacaacaacagcagacagtga

SEQ ID NO: 10 - DNA, 2391 bp, Artificial Sequence
  synthetic nucleotide
atgcctacaacacagcagtcccctcaggatgagcaggaaaagctcttggatgaagccatacaggctgtgaaggtccagtcattccaaatgaagagatgcctggacaaaaacaagctcatggatgctctaaaacatgcttctaatatgcttggtgaactccggacttctatgttatcaccaaagagttactatgaactttatatggccatttctgatgaactgcactacttggaggtctacctgacagatgagtttgctaaaggaaggaaagtggcagatctctacgaacttgtacagtatgctggaaacattatcccaaggctttaccttctgatcacagttggagttgtatatgtcaagtcatttcctcagtccaggaaggatattctgaaagatttggtagaaatgtgccgtggtgtgcaacatcccttgaggggtctgtttcttcgaaattaccttcttcagtgtaccagaaatatcttacctgatgaaggagagccaacagatgaagaaacaactggtgacatcagtgattccatggatttcgtactgctcaactttgcagaaatgaacaagctctgggtgcgaatgcagcatcagggacatagccgagatagagaaaaaagagaacgagaaagacaagaactgaggattctagtgggaacaaatttggtgcgcctcagtcagttggaaggtgtaaatgtggaacgttacaaacagattgttctgactggcatattggagcaagttgtaaactgtagggatgctttggctcaagaatatctcatggagtgtattattcaggtattccctgatgaatttcacctccagactttgaatcctttccttcgggcctgtgctgagttacaccagaatgtaaatgtgaagaacataatcattgctttaattgatagattagctttatttgctcaccgtgaagatggacctggaatcccagcggatattaaactattcgatatattctcacagcaggtggctacagtgatacagtcaagacaagacatgccttcagaggatgttgtatctttacaagtatctcttattaatcttgccatgaaatgttaccctgatcgtgtggactatgttgataaagtactagaaacaacagtggagatattcaataagctcaaccttgaacatattgctaccagtagtgcagtttcaaaggaactcaccagactattgaaaataccagttgacacttacaacaatatattaacagtcttgaaattaaaacatttccacccactctttgagtactttgactacgagtccagaaagagcatgagttgttatgtgcttagtaatgttctggattataacacagaaattgtatctcaagaccaggtggattccataatgaatttggtatccacgttgattcaagatcagccagatcaacctgtagaagaccctgatccagaagatttcgctgatgagcagagccttgtgggccgcttcattcatctgctgcgctctgaggaccctgaccagcagtacttgatattgaacacagcacgaaaacatttcggagctggtggaaatcagcggattcgcttcacactgccacctttggtatttgcagcttaccagctggcatttcgatataaagagaactctaaagtggatgacaaatgggaaaagaaatgccagaagatattctcatttgcccaccagactatcagtgctttgatcaaagcagagctggcagaattgccgttaagactattccttcaaggagcactagctgctggggaaattggatttgaaaatcatgaaacagtcgcatatgaattcatgtcccaggcattctctctgtatgaagatgaaatcagcgattccaaagcacagcttgctgccatcaccttgatcattggcacatttgaaaggatgaagtgcttcagtgaagagaatcatgaacctctgaggactcagtgtgcccttgctgcatccaaacttctaaagaaacctgatcagggccgagctgtgagcacctgtgcacatctcttctggtctggcagaaacacggacaaaaatggggaggagcttcacggaggcaagagggtaatggagtgcctaaaaaaagctctaaaaatagcaaatcagtgcatggacccctctctacaagtgcagctattcatagaaattctgaacagatatatctacttctatgaaaaggaaaatgatgcggtaacaattcaggtattaaaccagcttatccaaaagattcgagaagacctcccgaatcttgaatccagtgaagaaacagagcagattaacaaacactttcataacacactggagcatttgcgcttgcggcgggaatcaccagaatccgaggggccaatttatgaaggactcatcctttaa

SEQ ID NO: 11 - protein, 15 aa, Artificial Sequence
  synthetic peptide
SGYSSPGSPGTPGSR